SEQUENCE LISTING (60 sequences)

SEQ ID NO: 1 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRRIKILASSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTGGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPAKHKYDAEYYIENQVLPAVERILRAFGYRKEDLRYQKTRQVGLSAWLKPKGT

SEQ ID NO: 2 - protein, 775 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDRIRAHPAVVDIYEYDIPFAKRYLIDKGLIPMEGDEELTMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGKPKEKVYAEEIAQAWETGEGLERVARYSMEDAKVTYELGKEFLPMEAQLSRLVGHPLWDVSRSSTGNLVEWYLLRKAYERNELAPNKPDEREYERRLRESYAGGYVKEPEKGLWENIVYLDFRSLYPSIIITHNVSPDTLNREGCKEYDVAPQVGHRFCKDFPGFIPSLLGDLLEERQKIKRKMKATVDLLEKKLLDYRQRRIKILASGYYGYYGYARARWYCKECAESVTAWGRQYIETTIREIEEKFGFKVLYADTDGFFAPIHGADAETVKKKAKEFLDYINAKLPGLLELEYEGFYKRGFFVTKKKYAVIDEEDKITTGGLEIVRRGWSEIAKETQARVLEALLKDGDVEKAVRIVKEVTEKLSKYEVPPEKLVIHEQITRDLRDYKATGPHVAVAKRLAARGVKIRPGTVISYIVLKGSGRIGDRAISFDEFDPAKHKYDAEYYIENQVLPPVERILRACGYRKEDLRYQKTRQVGLSAWLTPKGT

SEQ ID NO: 3 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 4 - DNA, 2325 bp, synthetic construct
  source 1..2325: mol_type = other DNA
atgatcctcgacactgactacataaccgaggatggaaagcctgtcataagaattttcaagaaggaaaacggcgagtttaagattgagtacgaccggacttttgaaccctacttctacgccctcctgaaggacgattctgccattgaggaagtcaagaagataaccgccgagaggcacgggacggttgtaacggttaagcgggttgaaaaggttcagaagaagttcctcgggagaccagttgaggtctggaaactctactttactcatccgcaggacgtcccagcgataagggacaagatacgagagcatccagcagttattgacatctacgagtacgacatacccttcgccaagcgctacctcatagacaagggattagtgccaatggaaggcgacgaggagctgaaaatgctcgccttcgcgattgcgactctctaccatgagggcgaggagttcgccgaggggccaatccttatgataagctacgccgacgaggaaggggccagggtgataacttggaagaacgtggatctcccctacgttgacgtcgtctcgacggagagggagatgataaagcgcttcctccgtgttgtgaaggagaaagacccggacgttctcataacctacaacggcgacaacttcgacttcgcctatctgaaaaagcgctgtgaaaagctcggaataaacttcgccctcggaagggatggaagcgagccgaagattcagaggatgggcgacaggtttgccgtcgaagtgaagggacggatacacttcgatctctatcctgtgataagacggacgataaacctgcccacatacacgcttgaggccgtttatgaagccgtcttcggtcagccgaaggagaaggtttacgctgaggaaataaccacagcctgggaaaccggcgagaaccttgagagagtcgcccgctactcgatggaagatgcgaaggtcacatacgagcttgggaaggagttccttccgatggaggcccagctttctcgcttaatcggccagtccctctgggacgtctcccgctccagcactggcaacctcgttgagtggttcctcctcaggaaggcctatgagaggaatgagctggccccgaacaagcccgatgaaaaggagctggccagaagacggcagagctatgaaggaggctatgtaaaagagcccgagagagggttgtgggagaacatagtgtacctagattttagatccctgtacccctcaatcatcatcacccacaacgtctcgccggatacgctcaacagagaaggatgcaaggaatatgacgttgccccacaggtcggccaccgcttctgcaaggacttcccaggatttatcccgagcctgcttggagacctcctagaggagaggcagaagataaagaagaagatgaaggccacgattgacccgatcgagaggaagctcctcgattacaggcagaggcgtatcaagatcctggcaagcagctactacggttactacggctatgcaagggcgcgctggtactgcaaggagtgtgcagagagcgtaacggcctggggaagggagtacataacgatgaccatcaaggagatagaggaaaagtacggctttaaggtaatctacagcgacaccgacggattttttgccacaatacctggagccgatgctgaaaccgtcaaaaagaaggctatggagttcctcaagtatatcaacgccaaacttccgggcgcgcttgagctcgagtacgagggcttctacaaacgcggcttcttcgtcacgaagaagaagtatgcggtgatagacgaggaaggcaagataacaacgggtggacttgagattgtgaggcgtgactggagcgagatagcgaaagagacgcaggcgagggttcttgaagctttgctaaaggacggtgacgtcgagaaggccgtgaggatagtcaaagaagttaccgaaaagctgagcaagtacgaggttccgccggagaagctggtgatccacgagcagataacgagggatttaaaggactacaaggcaaccggtccccacgttgccgttgccaagaggttggccgcgagaggagtcaaaatacgccctggaacggtgataagctacatcgtgctcaagggctctgggaggataggcgacagggcgataccgttcgacgagttcgacccggcgaagcacaagtacgacgccgagtactacattgagaaccaggttctcccagccgttgagagaattctgagagccttcggttaccgcaaggaagacctgcgctaccagaagacgagacaggttggtttgagtgcttggctgaagccgaagggaacttga

SEQ ID NO: 5 - DNA, 2367 bp, synthetic construct
  source 1..2367: mol_type = other DNA
agcggcctggtgccgcgcggcagcatgatcctcgacactgactacataaccgaggatggaaagcctgtcataagaattttcaagaaggaaaacggcgagtttaagattgactacgaccggaactttgaaccctacatctacgccctcctgaaggacgattctgccattgaggacgtcaagaagataaccgccgagaggcacgggacgaccgtaagggttgtccgggccgaaaaggttaagaagaagttcctcgggagaccaatagaggtctggaaactctactttactcatccgcaggacgtcccagcgataagggacaagataaaggagcatccagcagttgtggacatctacgagtacgacatacccttcgccaagcgctacctcatagacaagggattaatcccaatggaaggcgacgaggagctgaaaatgctcgccttcgcgattgcgactctctaccatgagggcgaggagttcgccgaggggccaatccttatgataagctacgccgacgaggaaggggccagggtgataacttggaagaacatcgatctcccctacgttgacgtcgtctcgacggagaaggagatgataaagcgcttcctcaaggttgtgaaggagaaagacccggacgttctcataacctacaacggcgacaacttcgacttcgcctatctgaaaaagcgctccgaaaagctcggagtcaagttcatcctcggaagggaaggaagcgagccgaagattcagaggatgggcgacaggtttgccgtcgaagtgaagggacggatacacttcgatctctatcctgtgataagacggacgataaacctgcccacatacacgcttgaggccgtttatgaagccatcttcggtcagccgaaggagaaggtttacgctgaggaaatagcgcaggcctgggaaaccggcgagggacttgagagagtcgcccgctactcgatggaagatgcgaaggtcacatacgagcttgggaaggagttcttcccgatggaggcccagctttctcgcttagtaggccagtccctctgggacgtctcccgctccagcactggcaacctcgttgagtggttcctcctcaggaaggcctatgagaggaatgagctggccccgaacaagcccgatgaaagggagctggccagaagacgggagagctatgcgggaggctatgtaaaagagcccgagagagggttgtgggagaacatagtgtacctagattttagatccctgtacccctcaatcatcatcacccacaacgtctcgccggatacgctcaacagagaaggatgcgaggaatatgacgttgccccacaggtcggccacaagttctgcaaggacttcccaggatttatcccgagcctgcttggagacctcctagaggagaggcagaaggtaaagaagaagatgaaggccacgattgacccgatcgagaagaagctcctcgattacaggcagaggcgtatcaagatcctggcaagcagcttctacggttactacggctatgcaaaggcgcgctggtactgcaaggagtgtgcagagagcgtaacggcctggggaaggcagtacatagagaccaccatcagggagatagaggaaaagtttggctttaaggtactctacgcggacaccgacggattttttgccacaatacctggagccgatgctgaaaccgtcaaaaagaaggctaaggagttcctcgactatatcaacgccaaacttccgggcctgcttgagctcgagtacgagggcttctacaaacgcggcttcttcgtcacgaagaagaagtatgcggtgatagacgaggaagacaagataacaacgggtggacttgagattgtgaggcgtgactggagcgagatagcgaaagagacgcaggcgagggttcttgaagctatactaaagcacggtgacgtcgaggaagccgtgaggatagtcaaagaagttaccgaaaagctgagcaagtacgaggttccgccggagaagctggtgatctacgagcagataacgagggatttaaaggactacaaggcaaccggtccccacgttgccgttgccaagaggttggccgcgagaggaataaaaatacgccctggaacggtgataagctacatcgtgctcaagggctctgggaggataggcgacagggcgataccgttcgacgagttcgacccggcgaagcacaagtacgacgccgagtactacattgagaaccaggttctcccagccgttgagagaattctgagagccttcggttaccgcaaggaagacctgcgctaccagaagacgagacaggttggtttgggggcttggctgaagccgaagacatgagtcgacctgcagccaagctta

SEQ ID NO: 6 - DNA, 2373 bp, synthetic construct
  source 1..2373: mol_type = other DNA
agcggcctggtgccgcgcggcagcatgatcctcgacgctgactacataaccgaggatggaaagcctattataagaattttcaagaaggaaaacggcgagtttaaggttgagtacgaccggaactttagaccctacatttacgccctcctgaaggacgattctcagattgatgaagtcaggaagataaccgccgagaggcacgggaagatagtaagaattatagatgccgaaaaggttaggaagaagttcctcgggagaccaattgaggtctggaggctctactttgaacatccgcaggacgtcccagcgataagggacaagatacgagagcattccgcagttattgacatctttgagtacgacatacccttcgccaagcgctacctcatagacaagggattaattccaatggaaggcgacgaggagctgaaattgctcgccttcgcgattgcgactctctaccatgagggcgaggagttcgccaaggggccaatcataatgataagctacgccgacgaggaagaagccaaagtgataacttggaagaagatcgatctcccctacgttgaggtcgtctcgagcgagagggagatgataaagcgcttcctcaaggttataagggagaaagacccggacgttataataacctacaacggcgactctttcgaccttccctatctggttaagcgcgccgaaaagctcggaataaagctacccctcggaagggatggaagcgagccgaagatgcagaggcttggcgacatgacagccgtcgaaataaagggacggatacacttcgatctctatcacgtgataagacggacgataaacctgcccacatacacgcttgaggccgtttatgaagccatcttcggtaagccgaaggagaaggtttacgctcacgaaatagctgaggcctgggaaaccggcaagggacttgagagagtcgccaagtactcgatggaagatgcgaaggtcacatacgagcttgggagggagttcttcccgatggaggcccagctttctcgcttagtcggccagcccctctgggacgtctcccgctccagcactggcaacctcgttgagtggtacctcctcaggaaggcctatgagaggaatgagctggccccgaacaagcccgatgaaagggagtacgagagaaggctacgggagagctatgctggaggctatgtaaaagagcccgagaaagggttgtgggaggggttagtgtccctagattttagatccctgtacccctcaatcatcatcacccacaacgtctcgccggatacgctcaacagagaaggatgcagggaatatgacgttgccccagaggtcggccacaagttctgcaaggacttcccaggatttatcccgagcctgcttaagaggctcctagatgagaggcaggaaataaagaggaagatgaaggcctctaaagacccgatcgagaagaagatgctcgattacaggcagaggcgtatcaagatcctggcaagcagctactacggttactacggctatgcaaaagcgcgctggtactgcaaggagtgtgcagagagcgtaacggcctggggaagggagtacatagagttcgtaaggaaggagctggaggaaaagttcggctttaaggtattatacatagacaccgacggactctacgccacaatacctggagccaaacccgaagagataaaaaagaaggctctagagttcgtagattatatcaacgccaaacttccgggcctgcttgagctcgagtacgagggcttctacgtgcgcggcttcttcgtcacgaagaagaagtatgcgttgatagacgaggaaggcaagataatcacgggtggacttgagattgtgaggcgtgactggagcgagatagcgaaagagacgcaggcgaaagttcttgaagctatcctaaagcatggtaacgtcgaggaggccgtgaagatagtcaaagaagttaccgaaaagctgagcaagtacgagataccgccggagaagctggtgatctacgagcagataacgaggcccttacacgagtacaaggcaataggtccccacgttgccgttgccaagaggttggccgcgagaggagtcaaagtgcgccctggaatggtgatagggtacatcgtgctcaggggcgacgggccaataagcaagagggcgatacttgcagaggagttcgacctcgcgaagcacaagtacgacgccgagtactacattgagaaccaggttctcccagccgttcttagaattctggaggccttcggttaccgcaaggaagacctgcgctggcagaagacgaaacagacaggtttgacggcttggctgaacatcaagaagaagtgagtcgacctgcagccaagctta

SEQ ID NO: 7 - DNA, 2370 bp, synthetic construct
  source 1..2370: mol_type = other DNA
agcggcctggtgccgcgcggcagcatgatcctcgacactgactacataaccgagaacggaaagcctgtcataagagtgttcaagaaggaaaacggcgagtttaagattgagtacgaccggacttttgaaccctacttctacgccctcctgaaggacgattctgccattgaggatgtcaagaaggtgaccgccaaaaggcacgggacggttgtaaaagttaagcgggcggaaaaggttcagaagaagttcctcgggagaccaattgaggtctggaaactctactttaaccatccgcaggacgtcccagcgataagggaccgtatacgagcgcatccagcagttgtggacatctacgagtacgacatacccttcgccaagcgctacctcatagacaagggattaattccaatggaaggcgacgaggagctgaccatgctcgccttcgcgattgcgactctctaccatgagggcgaggagttcggcaccgggccaatccttatgataagctacgccgacggcagcgaagccagggtgataacttggaagaaaatcgatctcccctacgttgacgtcgtctcgacggagaaagagatgataaagcgcttcctccgtgttgtgcgtgagaaagacccggacgttctcataacctacaacggcgacaacttcgacttcgcctatctgaaaaagcgctgtgaagaactcggaataaaattcaccctcggaagggatggaagcgagccgaagattcagaggatgggcgacaggtttgccgtcgaagtgaagggacggatacacttcgatctctatcctgtgataagacggacgataaacctgcccacatacacgcttgaggccgtttatgaagccgtcttcggtaaaccgaaggagaaggtttacgctgaggaaatagcgcaggcctgggaaagcggcgagggccttgagagagtcgcccgctactcgatggaagatgcgaaggtcacatacgagcttgggcgtgagttcttcccgatggaggcccagctttctcgcttaatcggccagtccctctgggacgtctcccgctccagcactggcaacctcgttgagtggttcctcctcaggaaggcctataaaaggaatgagctggccccgaacaagcccgatgaacgtgagctggccagacgtcgtggcggttatgcgggaggctatgtaaaagagcccgagagagggttgtgggataacatagtgtacctagattttagatccctgtacccctcaatcatcatcacccacaacgtctcgccggatacgctcaacagagaaggatgcaaggaatatgacgttgccccagaagtcggccacaaattctgcaaggacttcccaggatttatcccgagcctgcttggagacctcctagaggagaggcagaagataaagcgcaagatgaaggccacggttgacccgctggagaaaaagctcctcgattacaggcagaggcgtatcaagatcctggcaagcagcttctacggttactacggctatgcaaaagcgcgctggtactgcaaggagtgtgcagagagcgtaacggcctggggaagggagtacatagaaatggtgatccgcgagctggaggaaaagttcggctttaaggtactgtacgcggacaccgacggactgcatgccacaatacctggagccgatgctgaaaccgtcaaaaagaaggctaaagagttcctcaagtatatcaacccgaaacttccgggcctgcttgagctcgagtacgagggcttctacgtgcgcggcttcttcgtcacgaagaagaagtatgcggtgatagacgaggaaggcaagataacaacgggtggacttgagattgtgaggcgtgactggagcgagatagcgaaagagacgcaggcgagggttcttgaagctattctaaagcatggtgacgtcgaggaagccgtgaggatagtcaaagaagttaccgaaaagctgagcaagtacgaggttccgccggagaagctggtgatccacgagcagataacgagggatttacgtgactacaaggcaaccggtccccacgttgccgttgccaagaggttggccgcgagaggagtcaaaatacgccctggaacggtgataagctacatcgtgctcaagggctctgggaggataggcgacagggcgataccggcggacgagttcgacccggcgaagcaccgttacgacgccgagtactacattgagaaccaggttctcccagccgttgagagaattctgaaagccttcggttaccgcaaggaagacctgcgctaccagaagacgaaacaggttggtttgggcgcttggctgaaggttaagggaaaatgagtcgacctgcagccaagctta

SEQ ID NO: 8 - DNA, 1395 bp, synthetic construct
  source 1..1395: mol_type = other DNA
gagttccttccgatggaggcccagctttctcgcttaatcggccattccctctgggacgtctcccgctccagcactggcaacctcgttgagtggttcctcctcaggaaggcctatgagaggaatgagctggccccgaacaagcccgatgaaaaggagctggccagaagacggcagagctatgaaggaggctatgtaaaagagcccgagagagggttgtgggagaacatagtgtacctagattttagatccctgtacccctcaatcatcatcacccacaacgtctcgccggatacgctcaacagagaaggatgcaaggaatatgacgttgccccacaggtcggccaccgcttctgcaaggacttcccaggatttatcccgagcctgcttggagacctcctagaggagaggcagaagataaagaagaagatgaaggccacgattgacctgatcgagaggaagctcctcgattacaggcagaggcgtatcaagatcctggcaagcggctactacggttactacggctatgcaagggcgcgctggtactgcaaggagtgtgcagagagcgtaacggcctggggaagggagtacataacgatgaccatcaaggagatagaggaaaagtacggctttaaggtaatctacagcgacaccgacggattttttgccccaatacatggagccgatgctgaaaccgtcaaaaagaaggctatggagttcctcaagtatatcaacgccaaacttccgggcgcgcttgagctcgagtacgagggcttctacaaacgcggcttcttcgtcacgaagaagaagtatgcggtgatagacgaggaaggcaagataacaacgggtggacttgagattgtgaggcgtggctggagcgagatagcgaaagagacgcaggcgagggttcttgaagctttgctaaaggacggtgacgtcgagaaggccgtgaggatagtcaaagaagttaccgaaaagctgagcaagtacgaggttccgccggagaagctggtgatccacgagcagataacgagggatttaaaggactacaaggcaaccggtccccacgttgccgttgccaagaggttggccgcgagaggagtcaaaatacgccctggaacggtgataagctacatcgtgctcaagggctctgggaggataggcgacagggcgatatcgttcgacgagttcgacccggcgaagcacaagtacgacgccgagtactacattgagaaccaggttctcccacccgttgagagaattctgagagcctgcggttaccgcaaggaagacctgcgctaccagaagacgagacaggttggtttgagtgcttggctgacgccgaagggaacttgagtcgacctgcagccaagcttaattagctgagcttggactcct

SEQ ID NO: 9 - DNA, 35 bp, synthetic construct
  source 1..35: mol_type = other DNA
tgagtcgacctgcagccaagcttaattagctgagc

SEQ ID NO: 10 - DNA, 18 bp, synthetic construct
  source 1..18: mol_type = other DNA
gctgccgcgcggcaccag

SEQ ID NO: 11 - DNA, 27 bp, synthetic construct
  source 1..27: mol_type = other DNA
atccatatgatcctcgacactgactac

SEQ ID NO: 12 - DNA, 35 bp, synthetic construct
  source 1..35: mol_type = other DNA
acgcatgcggccgctcaagttcctttcggcgtcag

SEQ ID NO: 13 - DNA, 35 bp, synthetic construct
  source 1..35: mol_type = other DNA
acgcatgcggccgctcacttctggtagcgcaggtc

SEQ ID NO: 14 - DNA, 32 bp, synthetic construct
  source 1..32: mol_type = other DNA
acgcatgcggccgctcaaccgcaggctctcag

SEQ ID NO: 15 - DNA, 23 bp, synthetic construct
  source 1..23: mol_type = other DNA
atcaccatacggatccaagcggc

SEQ ID NO: 16 - DNA, 24 bp, synthetic construct
  source 1..24: mol_type = other DNA
tacgccctcctgaaggacgattct

SEQ ID NO: 17 - DNA, 24 bp, synthetic construct
  source 1..24: mol_type = other DNA
caggacgtcccagcgataagggac

SEQ ID NO: 18 - DNA, 23 bp, synthetic construct
  source 1..23: mol_type = other DNA
gccttcgcgattgcgactctcta

SEQ ID NO: 19 - DNA, 27 bp, synthetic construct
  source 1..27: mol_type = other DNA
aagggacggatacacttcgatctctat

SEQ ID NO: 20 - DNA, 21 bp, synthetic construct
  source 1..21: mol_type = other DNA
gaggcccagctttctcgctta

SEQ ID NO: 21 - DNA, 27 bp, synthetic construct
  source 1..27: mol_type = other DNA
ccggatacgctcaacagagaaggatgc

SEQ ID NO: 22 - DNA, 26 bp, synthetic construct
  source 1..26: mol_type = other DNA
ggcgtatcaagatcctggcaagcagc

SEQ ID NO: 23 - DNA, 27 bp, synthetic construct
  source 1..27: mol_type = other DNA
cttgagctcgagtacgagggcttctac

SEQ ID NO: 24 - DNA, 24 bp, synthetic construct
  source 1..24: mol_type = other DNA
gagatagcgaaagagacgcaggcg

SEQ ID NO: 25 - DNA, 21 bp, synthetic construct
  source 1..21: mol_type = other DNA
ccgccggagaagctggtgatc

SEQ ID NO: 26 - DNA, 24 bp, synthetic construct
  source 1..24: mol_type = other DNA
agaatcgtccttcaggagggcgta

SEQ ID NO: 27 - DNA, 24 bp, synthetic construct
  source 1..24: mol_type = other DNA
gtcccttatcgctgggacgtcctg

SEQ ID NO: 28 - DNA, 23 bp, synthetic construct
  source 1..23: mol_type = other DNA
tagagagtcgcaatcgcgaaggc

SEQ ID NO: 29 - DNA, 27 bp, synthetic construct
  source 1..27: mol_type = other DNA
atagagatcgaagtgtatccgtccctt

SEQ ID NO: 30 - DNA, 21 bp, synthetic construct
  source 1..21: mol_type = other DNA
taagcgagaaagctgggcctc

SEQ ID NO: 31 - DNA, 27 bp, synthetic construct
  source 1..27: mol_type = other DNA
gcatccttctctgttgagcgtatccgg

SEQ ID NO: 32 - DNA, 26 bp, synthetic construct
  source 1..26: mol_type = other DNA
gctgcttgccaggatcttgatacgcc

SEQ ID NO: 33 - DNA, 27 bp, synthetic construct
  source 1..27: mol_type = other DNA
gtagaagccctcgtactcgagctcaag

SEQ ID NO: 34 - DNA, 24 bp, synthetic construct
  source 1..24: mol_type = other DNA
cgcctgcgtctctttcgctatctc

SEQ ID NO: 35 - DNA, 21 bp, synthetic construct
  source 1..21: mol_type = other DNA
gatcaccagcttctccggcgg

SEQ ID NO: 36 - DNA, 24 bp, synthetic construct
  source 1..24: mol_type = other DNA
taagcttggctgcaggtcgactca

SEQ ID NO: 37 - DNA, 52 bp, synthetic construct
  source 1..52: mol_type = other DNA
acaaccattatctagagcgattcgtataggtggtatccgaaaggataccacc

SEQ ID NO: 38 - DNA, 20 bp, synthetic construct
  source 1..20: mol_type = other DNA
gtccccttggggataccacc

SEQ ID NO: 39 - DNA, 60 bp, synthetic construct
  source 1..60: mol_type = other DNA
catacgcgagatacactgccagaaaaccgcgtgtctacgaggtggtatccccaaggggac

SEQ ID NO: 40 - DNA, 47 bp, synthetic construct
  source 1..47: mol_type = other DNA
tcgttctcggacggagagatattgaanggtggtatccccaaggggac

SEQ ID NO: 41 - DNA, 50 bp, synthetic construct
  source 1..50: mol_type = other DNA
cccacaccctcctatcgctaaacacacacttaataaagttggtggtatcc

SEQ ID NO: 42 - DNA, 50 bp, synthetic construct
  source 1..50: mol_type = other DNA
aacaaagtatcgagagactgacgaaaaacataagagtagcggtggtatcc

SEQ ID NO: 43 - DNA, 30 bp, synthetic construct
  source 1..30: mol_type = other DNA
ggcgaattactaacagcgcgggtggtatcc

SEQ ID NO: 44 - DNA, 50 bp, synthetic construct
  source 1..50: mol_type = other DNA
acgcgcttcttggaaactcacgcacccataataatactaaggtggtatcc

SEQ ID NO: 45 - DNA, 40 bp, synthetic construct
  source 1..40: mol_type = other DNA
attgcttgccggagcttatcggaagactcacaggcctccg

SEQ ID NO: 46 - DNA, 20 bp, synthetic construct
  source 1..20: mol_type = other DNA
tcgttctcggacggagagat

SEQ ID NO: 47 - DNA, 20 bp, synthetic construct
  source 1..20: mol_type = other DNA
attgcttgccggagcttatc

SEQ ID NO: 48 - DNA, 81 bp, synthetic construct
  source 1..81: mol_type = other DNA; note = inverted dT
  misc_feature 81
tcgttctcggacggagagatacgcgcttcttggaaactcacgcacccataataatactaacggaggcctgtgagtcaacct

SEQ ID NO: 49 - DNA, 36 bp, synthetic construct
  source 1..36: mol_type = other DNA
attagctgagcttggactcctgttgatagatccagt

SEQ ID NO: 50 - DNA, 27 bp, synthetic construct
  source 1..27: mol_type = other DNA
ggcctccatcggaaggaactccttccc

SEQ ID NO: 51 - DNA, 10 bp, synthetic construct
  source 1..10: mol_type = other DNA
ggataccacc

SEQ ID NO: 52 - DNA, 50 bp, synthetic construct
  source 1..50: mol_type = other DNA
cctatggtggttgaaataattcacacacaaatcgctatcctcccacaccc

SEQ ID NO: 53 - DNA, 50 bp, synthetic construct
  source 1..50: mol_type = other DNA
ggataccaccaactttattaagtgtgtgtttagcgataggagggtgtggg

SEQ ID NO: 54 - protein, 774 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRRIKILASSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTGGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPAKHRYDAEYYIENQVLPAVERILKAFGYRKEDLRYQKTKQVGLGAWLKVKGK

SEQ ID NO: 55 - protein, 774 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRRIKILASSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTGGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPADEFDPAKHKYDAEYYIENQVLPAVERILRAFGYRKEDLRYQKTRQVGLGAWLKPKGT

SEQ ID NO: 56 - protein, 775 aa, synthetic construct
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFAIATLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRRIKILASSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITGGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLAKHKYDAEYYIENQVLPAVLRILEAFGYRKEDLRWQKTKQTGLTAWLNIKKK

SEQ ID NO: 57 - protein, 775 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDRIRAHPAVVDIYEYDIPFAKRYLIDKGLIPMEGDEELTMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGKPKEKVYAEEIAQAWETGEGLERVARYSMEDAKVTYELGKEFLPMEAQLSRLVGQPLWDVSRSSTGNLVEWYLLRKAYERNELAPNKPDEREYERRLRESYAGGYVKEPEKGLWENIVYLDFRSLYPSIIITHNVSPDTLNREGCKEYDVAPQVGHRFCKDFPGFIPSLLGDLLEERQKIKRKMKATVDPLEKKLLDYRQRRIKILASSYYGYYGYARARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTGGLEIVRRDWSEIAKETQARVLEALLKDGDVEKAVRIVKEVTEKLSKYEVPPEKLVIHEQITRDLRDYKATGPHVAVAKRLAARGVKIRPGTVISYIVLKGSGRIGDRAIPFDEFDPAKHKYDAEYYIENQVLPAVERILRAFGYRKEDLRYQKTRQVGLSAWLKPKGT

SEQ ID NO: 58 - protein, 775 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDRIRAHPAVVDIYEYDIPFAKRYLIDKGLIPMEGDEELTMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGKPKEKVYAEEIAQAWETGEGLERVARYSMEDAKVTYELGKEFLPMEAQLSRLVGQPLWDVSRSSTGNLVEWYLLRKAYERNELAPNKPDEREYERRLRESYAGGYVKEPEKGLWENIVYLDFRSLYPSIIITHNVSPDTLNREGCKEYDVAPQVGHRFCKDFPGFIPSLLGDLLEERQKIKRKMKATVDPLEKKLLDYRQRRIKILASSYYGYYGYARARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTGGLEIVRRGWSEIAKETQARVLEALLKDGDVEKAVRIVKEVTEKLSKYEVPPEKLVIHEQITRDLRDYKATGPHVAVAKRLAARGVKIRPGTVISYIVLKGSGRIGDRAISFDEFDPAKHKYDAEYYIENQVLPAVERILRACGYRKEDLRYQKTRQVGLSAWLTPKGT

SEQ ID NO: 59 - protein, 775 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDRIRAHPAVVDIYEYDIPFAKRYLIDKGLIPMEGDEELTMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGKPKEKVYAEEIAQAWETGEGLERVARYSMEDAKVTYELGKEFLPMEAQLSRLVGQPLWDVSRSSTGNLVEWYLLRKAYERNELAPNKPDEREYERRLRESYAGGYVKEPEKGLWENIVYLDFRSLYPSIIITHNVSPDTLNREGCKEYDVAPQVGHRFCKDFPGFIPSLLGDLLEERQKIKRKMKATVDPLEKKLLDYRQRRIKILASSYYGYYGYARARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTGGLEIVRRGWSEIAKETQARVLEALLKDGDVEKAVRIVKEVTEKLSKYEVPPEKLVIHEQITRDLRDYKATGPHVAVAKRLAARGVKIRPGTVISYIVLKGSGRIGDRAISFDEFDPAKHKYDAEYYIENQVLPPVERILRACGYRKEDLRYQKTRQVGLSAWLTPKGT

SEQ ID NO: 60 - DNA, 40 bp, synthetic construct
  source 1..40: mol_type = other DNA
acgcgcttcttggaaactcacgcacccataataatactaa

(19) United States
(12) Patent Application Publication
(10) Pub. No.: US 2026/0218140 A1
(43) Pub. Date: Jul. 30, 2026

(54) ENGINEERED POLYMERASE FOR THREOSE NUCLEIC ACID SYNTHESIS

(71) Applicant: THE REGENTS OF THE UNIVERSITY OF CALIFORNIA, OAKLAND, CA (US)

(72) Inventors: John C. Chaput, IRVINE, CA (US); Eric J. Yik, IRVINE, CA (US); Victoria A. Maola, IRVINE, CA (US)

(73) Assignee: THE REGENTS OF THE UNIVERSITY OF CALIFORNIA, OAKLAND, CA (US)

(21) Appl. No.: 19/146,471

(22) PCT Filed: Jan. 16, 2024
(86) PCT No.: PCT/US2024/011595

§ 371 Date: Jul. 8, 2025

Related U.S. Application Data
(60) Provisional application No. 63/480,049, filed on Jan. 16, 2023.

(51) Int. Cl. C12N 9/12; C12N 15/63; C12P 19/34

(52) U.S. Cl. CPC C12N 9/1252; C12N 15/63; C12P 19/34; C12Y 207/07007

(57) ABSTRACT

An improved threose nucleic acid (TNA) polymerase exhibits both increased efficiency and increased capability relative to earlier TNA polymerases, with a catalytic rate of ~1 nt/s and >99% fidelity. The new enzyme shows reduced activity against dNTPs, maintains ultrahigh thermal stability, and readily accepts C5-modified TNA nucleoside triphosphates (tNTPs). Also provided are methods for making and using the improved TNA polymerase. The TNA polymerase.

This application claims benefit of U.S. provisional patent application No. 63/480,049, filed Jan. 16, 2023, the entire contents of which are incorporated by reference into this application.

### STATEMENT REGARDING FEDERALLY SPONSORED RESEARCH

This invention was made with Government support under grant MCB 1946312 awarded by the National Science Foundation. The Government has certain rights in the invention.

### REFERENCE TO A SEQUENCE LISTING

The content of the XML file of the sequence listing named “UCI016_Seq”, which is 71 kb in size, created on Jan. 16, 2024, and electronically submitted herewith the application, is incorporated herein by reference in its entirety.

### BACKGROUND

Threose nucleic acid (TNA) is an artificial genetic polymer in which the natural five-carbon ribose sugar found in RNA has been replaced by an unnatural four-carbon threose sugar. TNA has become an important synthetic genetic polymer (XNA) due to its ability to efficiently base pair with complementary sequences of DNA and RNA. Unlike DNA and RNA, TNA is completely refractory to nuclease digestion, making it a promising nucleic acid analog for therapeutic and diagnostic applications.

Short TNA oligonucleotides were first constructed by automated solid-phase synthesis using phosphoramidite chemistry. More recently, polymerase engineering efforts have identified TNA polymerases that can copy longer stretches of genetic information back and forth between DNA and TNA using chemically synthesized TNA triphosphates as substrates. TNA replication occurs through a process that mimics RNA replication. In these systems, TNA is reverse transcribed into DNA, the DNA is amplified by the polymerase chain reaction, and then forward transcribed back into TNA.

The availability of TNA polymerases have enabled the in vitro selection of biologically stable TNA aptamers to both small molecule and protein targets. The high biological stability of TNA relative to other nucleic acid systems that are capable of undergoing Darwinian evolution, suggests that TNA is a strong candidate for the development of next-generation therapeutic aptamers.

Kod-RSGA, a previously developed DNA-dependent TNA polymerase, is able to transcribe individual strands or large libraries of degenerate DNA sequences into TNA. This property, which is remarkable considering the backbone structure of TNA relative to DNA and RNA, has enabled the evolution of TNA aptamers from unbiased pools of random sequences. The enzyme functions by a primer-extension mechanism in which a primer strand annealed to a DNA template is extended with chemically synthesized TNA triphosphates. Previous analyses indicate that Kod-RSGA functions with a modest rate of ~10 nucleotide per minute, which is ~1,000-fold slower than the rate of DNA synthesis by wild-type Kod DNA polymerase.

There remains a need for TNA polymerases with improved activity.

### SUMMARY

Described herein is an improved threose nucleic acid (TNA) polymerase, as well as methods for making and using the improved TNA polymerase. The TNA polymerase exhibits both increased efficiency and increased capability relative to earlier TNA polymerases.

In some embodiments, the threose nucleic acid (TNA) polymerase comprises an amino acid sequence at least 80% identical to SEQ ID NO: 2, wherein the amino acid sequence comprises each of the residues indicated as mutated or inserted relative to Kod-WT in FIG. 2A (R99, A102, V107, I127, T136, A141, A143, K285, A296, Q297, G304, V337, H339, P340, Y356, R375, Y377, E378, L381, E383, A386, K395, R466, V472, L474, L475, K477, R486, S492, G493, Q520, E523, T524, R527, F533, L538, A540, P548, H550, K562, D566, L575, D602, G607, G615, R672, S717, A724, P741, C749, and T771 of SEQ ID NO: 2); and wherein the encoded DNA polymerase synthesizes a TNA in the presence of a DNA template and threose nucleotides. In some embodiments, the amino acid sequence is at least 85% identical to SEQ ID NO: 2. In some embodiments, the amino acid sequence is at least 90% identical to SEQ ID NO: 2. In some embodiments, the threose nucleic acid (TNA) polymerase comprises an amino acid sequence at least 95% identical to SEQ ID NO: 2, wherein the amino acid sequence comprises each of the residues R99, A102, V107, I127, T136, A141, A143, K285, A296, Q297, G304, V337, H339, P340, Y356, R375, Y377, E378, L381, E383, A386, K395, R466, V472, L474, L475, K477, R486, S492, G493, Q520, E523, T524, R527, F533, L538, A540, P548, H550, K562, D566, L575, D602, G607, G615, R672, S717, A724, P741, C749, and T771 of SEQ ID NO: 2; and wherein the encoded DNA polymerase synthesizes a TNA in the presence of a DNA template and threose nucleotides. In some embodiments, the amino acid sequence is at least 98% identical to SEQ ID NO: 2. In some embodiments, the amino acid sequence is SEQ ID NO: 2. Where the amino acid sequence is less than 100% identical to SEQ ID NO: 2, each of the mutations/insertions relative to Kod-WT as indicated in FIG. 2A is present.

As shown in FIG. 2A, 10.92 TNA Polymerase (SEQ ID NO: 2) has 51 mutations (50 mutations and 1 insertion) relative to Kod-WT (SEQ ID NO: 3). Thus, in some embodiments in which the TNA polymerase shares less than 100% identity to SEQ ID NO: 2, the amino acid sequence comprises R at residue 99 (K99R), A at residue 102 (E102A), V at residue 107 (1107V), I at residue 127 (V127I), T at residue 136 (K136T), A at residue 141 (D141A), A at residue 143 (E143A), K at residue 285 (Q285K), A at residue 296 (T296A), Q at residue 297 (T297Q), G at residue 304 (N304G), V at residue 337 (1337V), H at residue 339 (Q339H), P at residue 340 (S340P), Y at residue 356 (F356Y), R at residue 375 (K375R), Y at residue 377 (L377Y), E at residue 378 (A378E), L at residue 381 (R380_R381insL), E at residue 383 (Q383E), A at residue 386 (E386A), K at residue 395 (R395K), R at residue 466 (K466R), V at residue 472 (1472V), L at residue 474 (P474L), L at residue 475 (1475L), K at residue 477 (R477K), R at residue 486 (A486R), S at residue 492 (N492S), G at residue 493 (S493G), Q at residue 520 (E520Q), E at residue 523 (T523E), T at residue 524 (M524T), R at residue 527 (K527R), F at residue 533 (Y533F), L at residue 538 (1538L), A at residue 540 (S540A), P at residue 548 (T548P), H at residue 550 (P550H), K at residue 562 (M562K), D at residue 566 (K566D), L at residue 575 (A575L), D at residue 602 (G602D), G at residue 607 (R607G), G at residue 615 (D615G), R at residue 672 (K672R), S at residue 717 (P717S), A at residue 724 (T724A), P at residue 741 (A741P), C at residue 749 (F749C), and T at residue 771 (K771T). Note that the inserted L at residue 381 results in a total length of 775 amino acids versus the 774 amino acid length of Kod-WT (SEQ ID NO: 3). The numbering used here is based on the 775 amino acids of 10.92 (SEQ ID NO: 2), whereas FIG. 2A shows numbering of Kod-RSGA mutations relative to Kod-WT based on the 774 amino length of Kod-WT, hence A485R, N491S, R608G, and T723A of FIG. 2A correspond to A486R, N492S, R608G, and T724A of 10.92.

In addition, described herein is a nucleic acid encoding a 10.92 TNA polymerase comprising an amino acid sequence at least 80% identical to SEQ ID NO: 2, wherein the amino acid sequence comprises each of the residues indicated as mutated or inserted relative to Kod-WT in FIG. 2A. In some embodiments, the nucleic acid is an expression vector. Also provided is a recombinant cell comprising the nucleic acid expression vector. In some embodiments, the nucleic acid sequence encodes the TNA polymerase of SEQ ID NO: 2 (Table 1).

Further described is a kit comprising the TNA polymerase and at least one threose nucleotide. In some embodiments, the at least one threose nucleotide comprises tA, tT, tG, and/or tC. In some embodiments, the kit further comprises one or more dNTPs. In some embodiments, the at least one threose nucleotide comprises tA, tT, tG, and tC.

Also described herein is a method for synthesizing a TNA. In some embodiments, the method comprises contacting a DNA template with a TNA polymerase as described herein and a plurality of threose nucleotides. The contacting occurs under conditions that permit TNA polymerization, whereby a TNA is synthesized by the TNA polymerase. In some embodiments, the amino acid sequence is at least 80% identical to SEQ ID NO: 2. In some embodiments, the amino acid sequence is at least 85%, 90%, 95%, or 98% identical to SEQ ID NO: 2. In some embodiments, the amino acid sequence is identical to SEQ ID NO: 2. Additionally described is a method for synthesizing a TNA that comprises base-modified nucleotides. In some embodiments, the method comprises contacting a DNA template with a TNA polymerase as described herein and a plurality of threose nucleotides, wherein at least one threose nucleotide triphosphate comprises a modified base. The contacting occurs under conditions that permit TNA polymerization, whereby a TNA comprising a modified base is synthesized by the TNA polymerase. In some embodiments, the modified base comprises a C5-modified tUTP monomer bearing a phenylalanine (Phe) or tryptophan (Trp) side chain. Other functional side chains can likewise be introduced at the C5 position. In some embodiments, the modified base comprises a C5-modified tUTP or tCTP monomer or C7-modified 7-deaza-tGTP or 7-deaza-tATP monomer bearing side chains. In some embodiments, the side chains are selected from phenylalanine (Phe), tryptophan (Trp), tyrosine, cyclopropyl, naphthalene, and isoleucine.

### BRIEF DESCRIPTION OF THE DRAWINGS

FIGS. 1A-1F. Homologous recombination as a path to engineered XNA polymerases. (1A) Chemical structure of the linearized backbone for natural DNA and 3′,2′-α-L-threofuranosyl nucleic acid (TNA) with accompanied bases. (1B) Crystal structure of Kod-RSGA TNA polymerase shaded by domain with the RSGA mutations shown as space-filling models (PDB: 7RSU). (1C) Structural representation of an example recombination product generated by DNA shuffling. Recombinant segments are shaded according to their parent gene, with conserved recombination sites numbered and shown in grey. (1D) Schematic illustration of a DNA shuffling approach applied to archaeal DNA polymerases. (1E) Overview of the microfluidic polymerase engineering process known as droplet-based optical polymerase sorting (DrOPS). Microfluidic devices are used for droplet generation and sorting. Intermediate steps of E. coli lysis and primer extension occur off-chip. (1F) Library assembly following iterative rounds of directed evolution involves Gibson assembly of PCR amplified genes with the backbone expression vector.

FIGS. 2A-2C. Neutral drift and directed evolution of a highly efficient TNA polymerase. (2A) Table summarizing the mutations that arose during the directed evolution of the 10-92 TNA polymerase. 5-270 is the product of DNA recombination and five rounds of selective enrichment, while 7-47, 8-64, and 10-92 are the products of directed evolution with intentional mutagenesis. (2B) Pie chart denoting the distribution of amino acid mutations found in 10-92. Total residues per domain (inner values) and mutations observed for each domain (outer values). (2C) Genomic map showing the recombination segments observed in 10-92 and amino acid mutations (dots) acquired following intentional mutagenesis and selection (top). Amino acid differences (solid circles) between Kod RSGA and 10-92 are mapped onto the crystal structure of Kod-RSGA (PDB: 7RSU) (bottom).

FIGS. 3A-3G. Functional analysis of TNA polymerases. (3A) Primer-extension assay. Sequence of the DNA primer-template duplex (left; SEQ ID NOs: 51, 52, upper and lower, respectively) before and after extension with TNA (right; SEQ ID NOs: 53, 52, upper and lower, respectively). (3B) Activity screen of TNA polymerases. Key enzymes identified during the evolution process were challenged to extend the DNA primer with 40 TNA residues in a reaction time of 2 minutes. (3C) Time course comparing the TNA synthesis activity of Kod-RSGA to 10-92. (3D) tNTP substrate dependency. Product formation was evaluated after 30 minutes of incubation with decreasing substrate concentrations. (3E) dNTP substrate specificity. Product formation was evaluated after 1 minute of incubation with decreasing substrate concentrations. (3F) Synthesis of functionally enhanced TNA. Product formation was compared after 30 minutes of incubation for tNTP mixtures containing C5-modified tUTP monomers bearing either phenylalanine (Phe) or tryptophan (Trp) side chains with amide linkages. (3G) Thermal stability. TNA synthesis activity was assessed after 1-6 hours of polymerase incubation at 90° C. Unless otherwise noted, reactions were performed by incubating a 5′ IR680-labeled DNA primer-template duplex (1 μM) with 1 μM polymerase and 100 μM tNTPs in 1× ThermoPol buffer at 55° C. C5-modified tNTP mixtures contain 2 μM polymerase. Primer-extension reactions were analyzed by denaturing polyacrylamide gel electrophoresis with fluorescent imaging on a LI-COR imager. P, primer; F, full-length product; M, marker.

FIG. 4. Kinetic analysis of TNA synthesis. Schematic overview. The rate of TNA synthesis was compared for the TNA polymerases Kod-RSGA and 10-92 using a fluorescence-based assay that measures nucleotide incorporation via dye intercalation into the growing duplex. Kinetic curves are shown for primer extension reactions catalyzed by Kod-RSGA (solid black diamonds) and 10-92 (hashed diamonds). Error bars represent the standard error of the mean (S.E.M) of 3 independent reaction replicates. Rates for the linear ranges are reported in nucleotides per polymerase per minute (Kod-RSGA: 2.26±0.11 nt/min and 10-92:54.35±1.04 nt/min).

FIG. 5. Enhanced functional activity. The polymerase 10-92 is superior to its predecessor Kod-RSGA at producing TNA aptamers, including those with Phe- and Trp-modified bases targeting diverse protein structures. BLI sensorgrams depict binding kinetics validate that the produced TNA aptamers bind their respective target.

FIGS. 6A-6C. Structural topology of the 10-92 TNA polymerase. (6A) Structural overlay of the closed ternary conformations of the 10-92 TNA polymerase (medium gray tone, PDB: 8T3X) and Kod DNA polymerase (transparent gray, PDB: 5OMF) reveal distinct conformational rearrangements (RMSD 2 Å) in the NTD and thumb regions of the enzyme. Secondary structural transitions observed in the (6B) thumb and (6C) palm impact duplex recognition. The palm subdomain contains the Leu381 insertion obtained by homologous recombination. Primer, template, and TNA substrates are depicted in background.

FIGS. 7A-7C. Active site of the 10-92 TNA polymerase. (7A) Surface rendering of the active site region of the 10-92 TNA polymerase trapped in a closed ternary conformation with a tATP substrate snugly fit in an active site cavity. Mutations observed in the active site pocket are indicated: K285, L475, L474, K477, V472. (7B) The tATP substrate is stabilized by direct contacts with residues in the finger subdomain and a magnesium ion (sphere), which is further coordinated by catalytic residues, D405 and D543 in the palm subdomain. Primer and template are shown in medium and darkest sticks, respectively. (7C) Comparison of the nascent base pair between the 10-92 and Kod-RI TNA polymerases. TNA and DNA nucleotides are shown as stick models, with a polder map of the tT^{d} and tATP substrates for 10-92 shown in gray mesh and contoured at 5.00.

FIG. 8. Table of local base-pair parameters (see Zheng, G., et al. Nucleic Acids Res. 37, W240-246, (2009).).

FIG. 9 Sequence alignment of archaeal RSGA DNA polymerase (SEQ ID NO: 1) variants (SEQ ID NOs: 54-56). Boxes indicate the positions previously discovered in Kod-RSGA (A485R, N491S, R606G, T723A) that are known to enhance TNA synthesis. Polymerases contain the exonuclease silencing mutations D141A and E143A. Conserved residues are denoted as (.). Natural variation from the Kod scaffold is represented by the single letter amino acid abbreviation. Alignment was generated in CLC Main Workbench.

FIG. 10 Assembly of the DNA recombination library by overlap PCR. Agarose gel showing assembly of the homologous recombination library. Lane L: DNA ladder. Lane 1: Kod-RSGA fragments 1-5. Lane 2: Kod-RSGA fragments 6-11. Lane 3: Kod-RSGA fragments 1-11. Lane 4: Kod-RSGA and Tgo-RSGA fragments 1-11. Lane 5: Kod-, Tgo-, DV-, and 9°N-RSGA fragments 1-11.

FIG. 11 Schematic of the lysate screen. Regenerated libraries are transformed into XL1 Blue E. coli for expression of enriched variants obtained from DrOPS. Single isolated colonies are picked, grown, and induced to express a variant of interest. Cells are harvested, suspended in buffer, subjected to heat lysis, and cold incubation to precipitate endogenous E. coli proteins. The cell debris is clarified by centrifugation. Lysate supernatant is added to a polymerase activity assay and quenched at designated time points. The reaction is quenched, denatured, and analyzed by denaturing PAGE. High performing variants are purified and characterized for activity.

FIG. 12. Lysate activity screen following five rounds of neutral selection. Library members present in the pool after five rounds of in vitro selection were individually screened for TNA synthesis activity in a primer-extension assay. A qualitative assessment of primer extension efficiency relative to Kod-RSGA is represented as a heatmap. Clone 5-270 is shown with a dot. The percent of overall activity (i.e., the sum of each rank) is represented on a bar graph.

FIG. 13. TNA synthesis activity of clone 5-270. Engineered polymerases Kod-RSGA and 5-270 challenged to incorporate TNA substrates in a primer extension assay, templated by a random DNA aptamer library (L30.5).

FIG. 14. Sequence alignment of newly evolved TNA polymerases (SEQ ID NOs: 57-59; 2). TNA polymerases are compared to Kod-RSGA (SEQ ID NO: 1). Conserved residues are denoted as (.), while mutations are denoted by their single letter amino acid abbreviations. The box denotes amino acid residues that are removed for structural studies. Alignment was generated in CLC Main Workbench.

FIG. 15. Phylogenetic analysis of the evolved TNA polymerases. MEGA was used to generate a phylogenetic tree by the neighbor-joining method of the newly evolved TNA polymerases relative to their starting parent sequences.

FIG. 16. Time course analysis of newly evolved TNA polymerases. Each polymerase variant was challenged to extend a DNA primer with 40 consecutive TNA residues. The reactions were quenched at defined time points and analyzed by denaturing polyacrylamide gel electrophoresis with fluorescent imaging on a LiCOR imager. P; primer, FL; full-length product.

FIG. 17. Functional analysis of 10-92. Time course analysis was performed with 10-92 and various tNTP mixtures to determine incorporation efficiency and relative rates across a 40 nt DNA template. Full length product with standard base tNTPs (top) can be observed after 30 sec, compared to 15 min for C5′-modified tUTPs phenylalanine (Phe) (middle) or tryptophan (Trp) side chains (bottom).

FIG. 18. Schematic of the fidelity assay. An overhang primer is designed with an AA-AA mismatch in the primer region of the DNA template (solid line) with a 3′ inverted deoxy-T. The 3′ inverted deoxy-T prevents extension into the overhang region. The TNA strand (dashed line) is synthesized across the DNA template by 10-92 TNA polymerase. The DNA-TNA strand is separated from the DNA template and reverse transcribed with Bst. The cDNA is amplified utilizing the overhang region as a primer binding site, TOPO cloned and sequenced. Sequences containing the TT-AA watermark demonstrates a round of writing and reading TNA.

FIG. 19. Polymerase fidelity data. Raw alignment of sangar sequencing results from a cycle of TNA replication by TNA polymerase 10-92 with standard base TNA triphosphates. The expected DNA sequence is written 5′→3′ (SEQ ID NO: 60). Correct nucleotide reads are denoted as (.) and deleted nucleotides are highlighted with hash marks and denoted as (-). From a study of 1000 nucleotide insertion events, the fidelity of the 10-92 TNA polymerase is 99.7% (0 misincorporations, 0 insertions, 3 deletions).

FIG. 20. Neutral drift validation. Kod-RSGA and 10-92 are compared to an engineered form of Kod-RSGA carrying the 10 beneficial mutations acquired by directed evolution. Each protein was challenged to copy a DNA template by extending TNA substrates to a DNA primer strand within 5 or 10 min of incubation at 55° C. Reactions were analyzed by denaturing polyacrylamide gel electrophoresis with fluorescent imaging on a LiCOR imager. P; primer, FL; full-length product

FIG. 21. Active site pocket volume. Pocket volumes were calculated using PyVOL, searching all pockets over a minimum volume threshold (500 Å) without partitioning. Probe min and max radii were set to 1.4 and 2.8, respectively. Metals, incoming tNTP triphosphates, and solvents were removed for measurement. The pocket of Kod-WT DNA polymerase (PDB: 5OMF), Kod-RI TNA polymerase (PDB: 5VU8), and 10-92Δ760 TNA polymerase (PDB: 8T3X) measure at 746 Å³, 1018 Å³, and 820 Å³, respectively.

### DETAILED DESCRIPTION

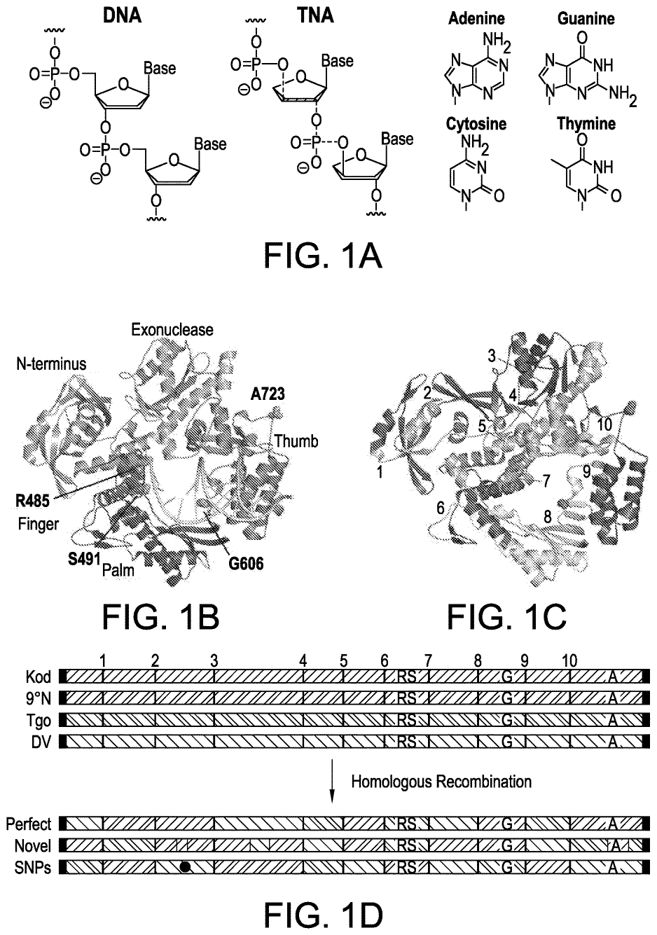

Described herein are improved TNA polymerases, nucleic acids encoding such TNA polymerases, kits comprising same, and methods for synthesizing TNAs using DNA as a template. The novel TNA polymerases described herein arose through the discovery that recombination can be used to reprogram DNA polymerases for the ability to recognize XNA triphosphates. For this purpose, α-L-threofuranosyl nucleic acid (TNA) was chosen as the target for enzymatic activity to allow comparison with previous efforts, and also to provide a stringent test of XNA substrate recognition, as the backbone repeat unit of TNA is one atom shorter than DNA and RNA (FIG. 1A). Starting from a carefully designed recombination library, we evolved a highly efficient TNA polymerase, termed 10-92, that functions with a catalytic rate of ~1 nt/s and >99% fidelity. The new enzyme shows reduced activity against dNTPs, maintains ultrahigh thermal stability, and readily accepts C5-modified TNA nucleoside triphosphates (INTPs). Consistent with fast enzyme kinetics, a crystal structure of the closed ternary complex capturing the primer-template duplex and tNTP substrate in a catalytically active conformation reveals a coplanar geometry for the incoming tNTP opposite its templating base. Structural alignment of the 10-92 TNA polymerase against its closest natural homolog highlights the extent of conformational change required to reprogram the biological function of a natural DNA polymerase for TNA synthesis activity. Together, these data demonstrate the power of this novel recombination strategy for providing XNA polymerases with improved activity that can be used as tools to synthesize artificial genetic polymers.

### Definitions

All scientific and technical terms used in this application have meanings commonly used in the art unless otherwise specified. As used in this application, the following words or phrases have the meanings specified.

As used herein, “TNA” or “TNAs” refer to nucleic acids having a backbone composed primarily of α-L-threofuranosyl-(3′→2′) (threose)-containing nucleotides, but may include heteropolymers comprising both tNTPs and dNTPs (e.g., dC).

As used herein, “TNTPs” refer to threose nucleotide triphosphates.

As used herein, “TNTP analog” refers to a threose nucleotide triphosphate having a modified base moiety.

As used herein, “TNA polymerase” refers to a polymerase capable of utilizing a DNA template and tNTPs to synthesize a complementary TNA sequence.

As used herein, “a” or “an” means at least one, unless clearly indicated otherwise.

### TNA Polymerases and Related Molecules

10.92 is a more active TNA polymerase than Kod-RSGA polymerase and can be used for all of the same applications previously described for other TNA polymerases. 10.92 is markedly better than either Therminator or Kod-RSGA in its ability to transcribe templates of increased complexity with higher efficiency. As a result, 10.92 can be used to generate more complex molecules with increasingly more sophisticated functions. 10.92 has potential to generate four nucleotide TNA molecules for in vitro selection of complex TNA molecules that are capable of performing complex functions, including molecules with modified bases designed for increased functional activity. Additionally, since biologically relevant molecules are usually comprised of four nucleotides, the ability to generate four nucleotide TNA molecules provides potential targeting mechanism for silencing technology. Additionally, there has been much interest in the information storage capabilities of nucleic acids. Since TNA is inherently nuclease resistant, TNA has the potential to become a highly stable and long lasting medium for storing large amounts of information.

In some embodiments the TNA polymerase comprises an amino acid sequence at least 80% (e.g., 85% or 90%) identical to the amino acid sequence of 10.92 polymerase shown below as SEQ ID NO: 2. In various embodiments the TNA polymerase comprises an amino acid sequence at least 95% (e.g., 97%, 98%, 99%, or 100%) identical to the amino acid sequence of 10.92 polymerase shown below as SEQ ID NO: 2.

In some embodiments, the TNA polymerase comprises one or more mutations relative to the amino acid sequence of SEQ ID NO: 2 and is greater than about 95% identical to the amino acid sequence of SEQ ID NO: 2. In one embodiment, the TNA polymerase to be used comprises the amino acid sequence of SEQ ID NO: 2.

The 10.92 TNA Polymerase (SEQ ID NO: 2) has 51 mutations (50 mutations and 1 insertion) relative to Kod-WT (SEQ ID NO: 3). Thus, in some embodiments in which the TNA polymerase shares less than 100% identity to SEQ ID NO: 2, the amino acid sequence comprises these 51 mutations: R at residue 99 (K99R), A at residue 102 (E102A), V at residue 107 (I107V), I at residue 127 (V127I), T at residue 136 (K136T), A at residue 141 (D141A), A at residue 143 (E143A), K at residue 285 (Q285K), A at residue 296 (T296A), Q at residue 297 (T297Q), G at residue 304 (N304G), V at residue 337 (I337V), H at residue 339 (Q339H), P at residue 340 (S340P), Y at residue 356 (F356Y), R at residue 375 (K375R), Y at residue 377 (L377Y), E at residue 378 (A378E), L at residue 381 (R380_R381insL), E at residue 383 (Q383E), A at residue 386 (E386A), K at residue 395 (R395K), R at residue 466 (K466R), V at residue 472 (I472V), L at residue 474 (P474L), L at residue 475 (I475L), K at residue 477 (R477K), R at residue 486 (A486R), S at residue 492 (N492S), G at residue 493 (S493G), Q at residue 520 (E520Q), E at residue 523 (T523E), T at residue 524 (M524T), R at residue 527 (K527R), F at residue 533 (Y533F), L at residue 538 (I538l), A at residue 540 (S540A), P at residue 548 (T548P), H at residue 550 (P550H), K at residue 562 (M562K), D at residue 566 (K566D), L at residue 575 (A575L), D at residue 602 (G602D), G at residue 607 (R607G), G at residue 615 (D615G), R at residue 672 (K672R), S at residue 717 (P717S), A at residue 724 (T724A), P at residue 741 (A741P), C at residue 749 (F749C), and T at residue 771 (K771T). Note that the inserted L at residue 381 results in a total length of 775 amino acids versus the 774 amino acid length of Kod-WT (SEQ ID NO: 3). The numbering used here is based on the 775 amino acids of 10.92 (SEQ ID NO: 2).

The TNA polymerase having at least 80% identity to the amino acid sequence of SEQ ID NO: 2 comprises the following residues, wherein the number indicates the position of the amino acid residue with respect to the 775 amino acid sequence shown in SEQ ID NO: 2, and the letter indicates the one-letter code for the amino acid residue present in that position: R99, A102, V107, I127, T136, A141, A143, K285, A296, Q297, G304, V337, H339, P340, Y356, R375, Y377, E378, L381, E383, A386, K395, R466, V472, L474, L475, K477, R486, S492, G493, Q520, E523, T524, R527, F533, L538, A540, P548, H550, K562, D566, L575, D602, G607, G615, R672, S717, A724, P741, C749, and T771.

TABLE 1
Sequence of 10.92 TNA Polymerase

TNA Polymerase 10.92 (protein, 775 aa; SEQ ID NO: 2)
MILDTDYITEDGKPVIRIFKKENGEFKIEYDRTFEPYFYALLKDDSAIEEVKKITAERHGTVVTVKRVEKV
QKKFLGRPVEVWKLYFTHPQDVPAIRDRIRAHPAVVDIYEYDIPFAKRYLIDKGLIPMEGDEELTMLAFAI
ATLYHEGEEFAEGPILMISYADEEGARVITWKNVDLPYVDVVSTEREMIKRFLRVVKEKDPDVLITYNGDN
FDFAYLKKRCEKLGINFALGRDGSEPKIQRMGDRFAVEVKGRIHFDLYPVIRRTINLPTYTLEAVYEAVFG
KPKEKVYAEEIAQAWETGEGLERVARYSMEDAKVTYELGKEFLPMEAQLSRLVGHPLWDVSRSSTGNLVEW
YLLRKAYERNELAPNKPDEREYERRLRESYAGGYVKEPEKGLWENIVYLDFRSLYPSIIITHNVSPDTLNR
EGCKEYDVAPQVGHRFCKDFPGFIPSLLGDLLEERQKIKRKMKATVDLLEKKLLDYRQRRIKILASGYYGY
YGYARARWYCKECAESVTAWGRQYIETTIREIEEKFGFKVLYADTDGFFAPIHGADAETVKKKAKEFLDYI
NAKLPGLLELEYEGFYKRGFFVTKKKYAVIDEEDKITTGGLEIVRRGWSEIAKETQARVLEALLKDGDVEK
AVRIVKEVTEKLSKYEVPPEKLVIHEQITRDLRDYKATGPHVAVAKRLAARGVKIRPGTVISYIVLKGSGR
IGDRAISFDEFDPAKHKYDAEYYIENQVLPPVERILRACGYRKEDLRYQKTRQVGLSAWLTPKGT

### TNA Polymerase Synthesis

Typically, TNA synthesis using the TNA polymerase is carried out at about 50° C. to about 60° C. In some embodiments, the TNA synthesis reaction is carried out at about 55° C.

Suitable concentrations of tNTPs range from about 100 μM to about 1000 μM, e.g., about 25, 30, 35, 40, 50, 60, 70, 80, or another concentration of tNTPs from about 100 μM to about 1000 μM.

In some embodiments, the single stranded DNA template to be used in the method comprises a sequence that is restricted to the nucleotides dA, dC, and dT. While not wishing to be bound by theory, it is believed that by limiting single stranded templates to sequences containing these three nucleotides, the fidelity of the sequence transcribed into TNAs is significantly increased. Also encompassed herein are heteropolymeric TNAs generated by the above-described method, which include tA, tT, tG, and dC.

Also described herein is method for reverse transcribing a TNA. In various embodiments, a TNA is reverse transcribed by a method that includes: contacting a TNA template that contains dCTP with Bst DNA polymerase in the presence of a primer and dNTPs, and incubating the resulting mix, at a temperature suitable for Bst DNA polymerase activity, to obtain a cDNA copy of the TNA template. One can also reverse transcribe sequences containing tCTP in the TNA strand. Typically the reverse transcription reaction using the Bst DNA polymerase is carried out at a temperature of about 37° C. to about 45° C. In some embodiments, the TNA reverse transcription reaction is carried out at 42° C. Also disclosed herein is a method for molecular evolution of threose nucleic acids, which includes the steps of: (i) providing a DNA template library containing diverse DNA template sequences; (ii) hybridizing the template library with one or more complementary primer sequences; (iii) incubating the hybridized template library with a DNA polymerase comprising an amino acid sequence at least 95% (e.g., 97%, 98%, 99%, or 100%) identical to the amino acid sequence of SEQ ID NO:2 in the presence of tTTP, tGTP, tATP, and dCTP, and incubating at a temperature suitable for polymerization by the DNA polymerase to obtain a cTNA library; (iv) subjecting the cTNA library to a selection assay to obtain at least one or more selected cTNAs; and (v) incubating the one or more selected cTNAs with a primer, a Bst DNA polymerase, and dNTPs at a temperature suitable for Bst DNA polymerase activity to obtain a selected DNA template library.

TNAs can be selected from a cTNA library in step (iv) based on a number of different criteria and assays depending on a desired functionality or endpoint for the TNAs being generated. Accordingly, in some embodiments the selection assay in step (iv) includes selection of one or more cTNAs from the cTNA library based on affinity for a ligand. Examples of suitable affinity assays known in the art include, but are not limited to, aptamer affinity chromatography, systematic evolution of ligands by exponential enrichment (SELEX), and kinetic capillary electrophoresis. In other embodiments, selection of one or more cTNAs from the cTNA library is based on a catalytic activity. Methods for assaying and selecting catalytic activities, e.g., ribozyme activities, are known in the art as described in, e.g., Link et al. (2007), Biol Chem 388 (8): 779-786. In some embodiments, one or more cTNAs are selected based on a desired fluorescence emission. See, e.g., Paige et at (2011), Science, 333 (6042): 642-646.

In the various methods described herein, hybridization between a primer and its target sequence is generally carried out under high stringency conditions under which the primer is annealed with its complementary template sequence at a temperature approximately 5° C. below the primer's melting temperature Tm.

### TNA Transcription Systems & Kits

Also described herein are TNA transcription systems. In various embodiments, a TNA transcription system includes the following components: a single stranded DNA template, a TNA polymerase comprising an amino acid sequence at least 95% identical to the amino acid sequence of 10.92, tTTP, tGTP, tATP; and (i) dCTP; or (ii) a combination of tCTP and dCTP.

Further described is a kit comprising the TNA polymerase and at least one threose nucleotide. In some embodiments, the at least one threose nucleotide comprises tA, tT, tG, and/or tC. In some embodiments, the kit further comprises one or more dNTPs. In some embodiments, the at least one threose nucleotide comprises tA, tT, tG, and tC.

### EXAMPLES

The following examples are presented to illustrate the present invention and to assist one of ordinary skill in making and using the same. The examples are not intended in any way to otherwise limit the scope of the invention.

### Example 1: Directed Evolution of a Novel DNA-Dependent TNA Polymerase Via Homologous Recombination

Reprogramming DNA polymerases to synthesize xeno-nucleic acids (XNA) is an important challenge that tests current enzyme engineering tools. This Example demonstrates an evolutionary campaign aimed at generating an XNA polymerase that can efficiently make α-L-threofuranosyl nucleic acid (TNA)—an artificial genetic polymer that is invisible to nucleases and resistant to acid-mediated degradation. Starting from a homologous recombination library, iterative cycles of selection were performed to traverse the fitness landscape in search of neutral mutations with increased evolutionary potential. Subsequent directed evolution yielded 10-92, a newly engineered TNA polymerase that functions with a catalytic rate of ~1 nt/s and >99% fidelity. A crystal structure of the closed ternary complex reveals the degree of structural change required to achieve efficient TNA synthesis. Together, these data demonstrate the importance of homologous recombination as a critical starting point for evolving XNA polymerases with considerable practical value for biotechnology and medicine.

Engineering DNA polymerases to synthesize artificial genetic polymers (also known as xeno-nucleic acids or XNAs) with high catalytic activity is an important challenge that tests current enzyme engineering strategies and offers a route to valuable reagents that can advance future applications in biotechnology and medicine¹. Although computational methods have solved many problems in protein design^{2,3}, attempts to create XNA polymerases by design have thus far proven unsuccessful, which is presumably due to the complicated reaction pathway that polymerases follow for each cycle of nucleotide addition⁴. We and others have attempted to explore this problem using high-throughput screening approaches that were designed to identify mutations that broaden the substrate specificity of natural DNA polymerases⁵⁻⁸. However, these attempts yielded enzymes with only modest activity⁹, indicating that the amino acid changes required to reprogram DNA polymerases with heightened levels of XNA synthesis activity remain mostly unknown. Uncovering the identity of these residues, which are likely to be unique for each XNA system, is a challenging combinatorial problem that will require deploying ultrahigh-throughput screening tools to search new regions of the sequence-fitness landscape for structural variants that are more adept at XNA synthesis.

Recognizing that polymerases are highly specific for their natural DNA or RNA substrates, we postulated that major structural rearrangements in the existing protein architecture would be required to engineer DNA polymerases for the ability to recognize XNA substrates with high catalytic activity and template-sequence fidelity. Although traditional mutagenic strategies, such as error-prone PCR (epPCR), saturation mutagenesis, and deep mutational scanning, are commonly used diversification techniques in protein engineering¹⁰, nature and laboratory evolution experiments have shown that higher levels of protein diversity are best achieved through the recombination of homologous protein fragments¹¹⁻¹³. Antibodies produced by the mammalian immune systems offer a notable example of how modularity is an efficient starting point for protein evolution¹⁴. In addition to increased structural diversity, recombination offers a more efficient approach for library generation because the resulting mutations are less disruptive than error-prone methods, thus resulting in a higher frequency of functional proteins¹⁵. Recombination also benefits from neutral drift, an important driving force in protein evolution that allows enzymes to traverse the fitness landscape in search of new mutations that function with increased evolutionary potential^{16,17}.

This disclosure shows how recombination can be used to reprogram DNA polymerases for the ability to recognize XNA triphosphates. For this study, α-L-threofuranosyl nucleic acid (TNA) was chosen as the target for enzymatic activity to allow comparison with previous efforts^{6,7} and also to provide a stringent test of XNA substrate recognition, as the backbone repeat unit of TNA is one atom shorter than DNA and RNA (FIG. 1a)¹⁸. Unlike other potentially natural derivatives of RNA¹⁹, TNA is capable of forming stable antiparallel duplex structures with itself and with complementary strands of DNA and RNA¹⁸. The unusual 2′,3′-linked phosphodiester backbone is invisible to nucleases²⁰ and stable against acid-mediated degradation²¹. In vitro selection experiments have shown that TNA is capable of folding into shapes with ligand binding and catalytic activity²²⁻²⁷, and its recognition by Bst DNA polymerase, a TNA reverse transcriptase, allows TNA to serve as a synthetic polymer for information storage²⁸. Finally, TNA has been evaluated as a building block for improving the biostability and activity of gene silencing applications involving DNAzymes²⁹, antisense oligonucleotides³⁰, and RNA interference pathways³¹.

Starting from a carefully designed recombination library, we evolved a highly efficient TNA polymerase, termed 10-92, that functions with a catalytic rate of ~1 nt/s and >99% fidelity. The new enzyme shows reduced activity against dNTPs, maintains ultrahigh thermal stability, and readily accepts C5-modified TNA nucleoside triphosphates (INTPs). Consistent with fast enzyme kinetics, a crystal structure of the closed ternary complex capturing the primer-template duplex and tNTP substrate in a catalytically active conformation reveals a coplanar geometry for the incoming tNTP opposite its templating base. Structural alignment of the 10-92 TNA polymerase against its closest natural homolog highlights the extent of conformational change required to reprogram the biological function of a natural DNA polymerase for TNA synthesis activity. Together, these data demonstrate the power of recombination as a general strategy for improving the activity of XNA polymerases that can be used as tools to synthesize artificial genetic polymers.

### Results

### Library Generation by Homologous Recombination

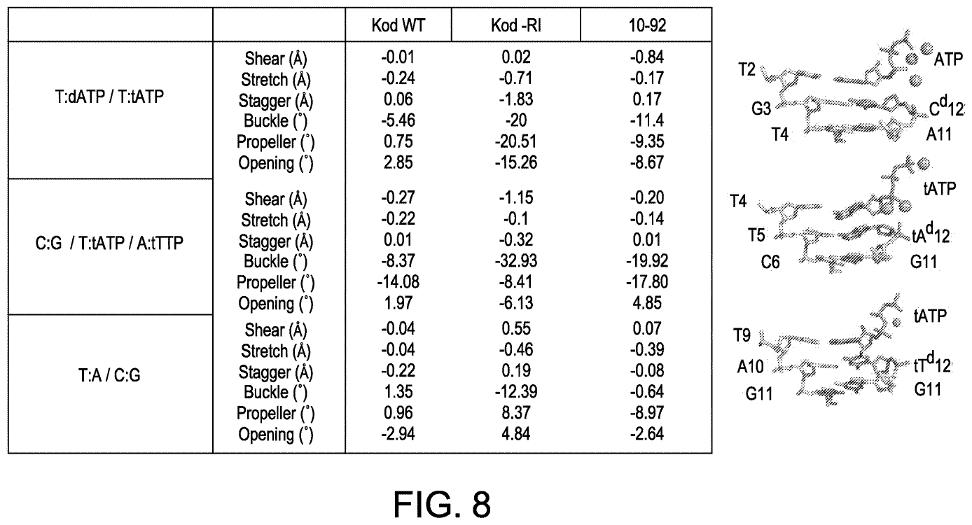

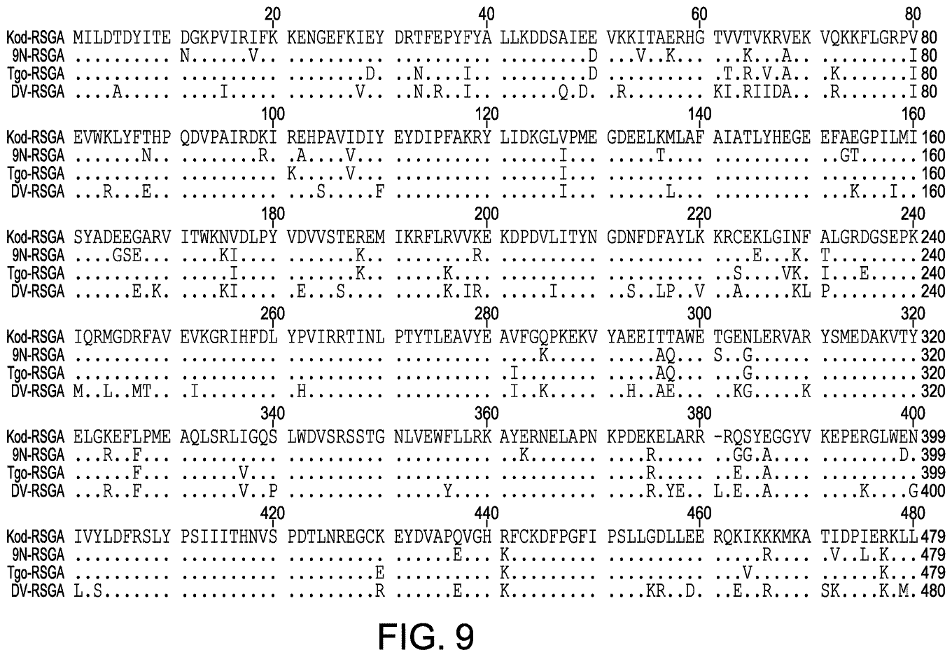

Because natural polymerases are constrained by a long history of evolutionary contingency, sequences with strong XNA synthesis activity are expected to be quite rare in sequence space³². We therefore chose to design our starting DNA library based on four hyperthermophilic archaeal B-family DNA polymerases, which are more accepting of modified nucleotides than other polymerase classes³³. Four 2.4 kilobase (kb) genes encoding polymerases isolated from the archaeal species Thermococcus kodakarensis (Kod), Thermococcus gorgonarius (Tgo), Pyrococcus Deep Vent (DV), and Thermococcus 9°N (9°N) were prepared as synthetic double-stranded (ds) DNA by high fidelity gBlock assembly (FIG. 9, Table 5, Table 6). Each gene carried the exonuclease silencing mutations (D141A and E143A), four beneficial mutations (A485R, N491S, R606G, and T723A) that derive from our previous best TNA polymerase, Kod-RSGA (FIG. 1b)⁷, and 10 conserved regions (FIG. 1c) that are required for synthetic recombination using the polymerase chain reaction (PCR). Homologous recombination (FIG. 1d) was achieved using a DNA shuffling approach in which 44 individual PCR reactions were performed to fragment each gene into 11 pieces that were reassembled in a single PCR reaction (FIG. 10) to create a pool of >4 million polymerase variants³⁴. DNA sequencing of individual clones isolated from the pool revealed a library composed of perfect recombinants, novel crossovers caused by template switching, and random mutations (SNPs). In contrast to previous polymerase libraries assembled by error-prone PCR (epPCR) or mutational scanning, this library was expected to explore a larger range of sequence space due to the divergent nature of the assembly process, which allowed for broader sampling of mutational and structural variants³⁵.

### Droplet-Based Polymerase Evolution

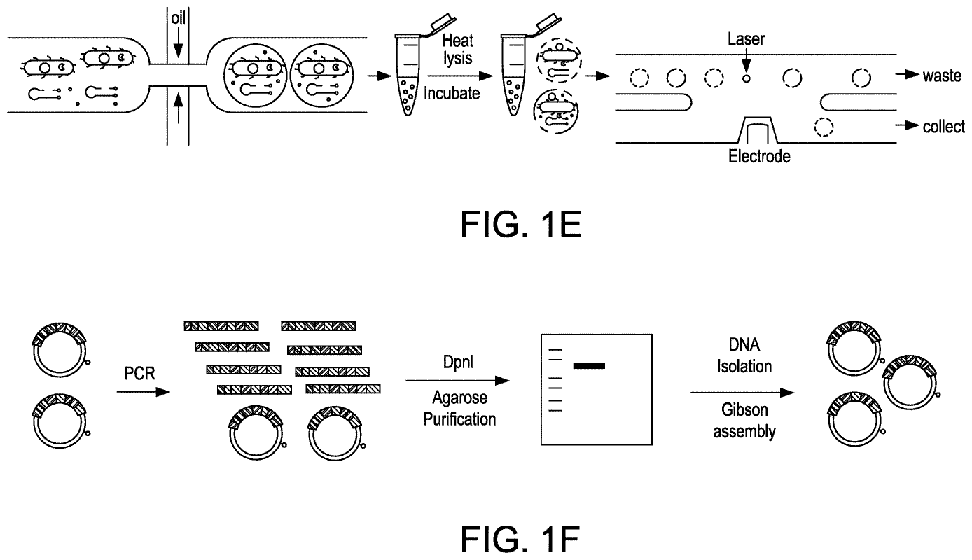

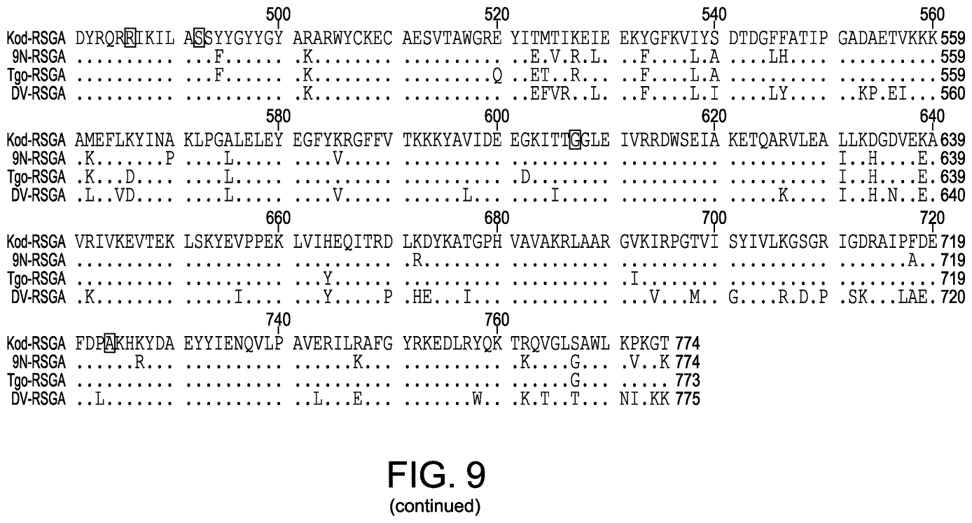

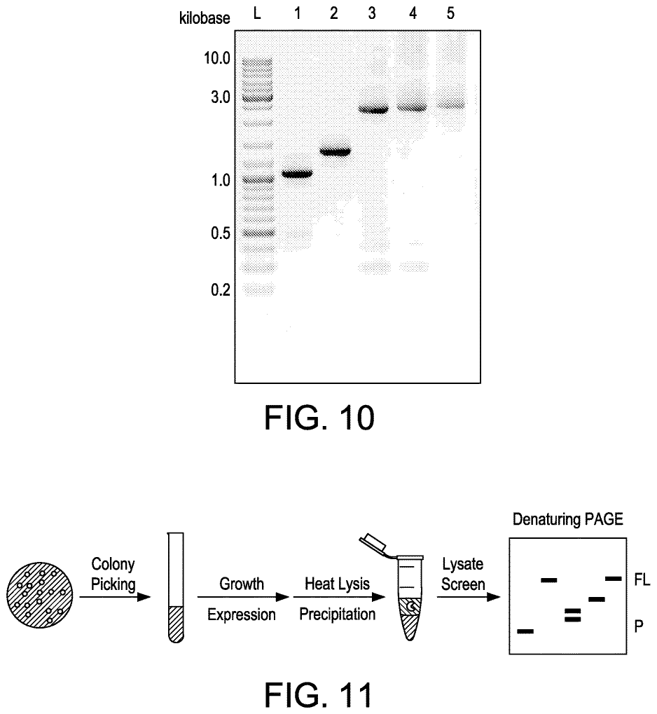

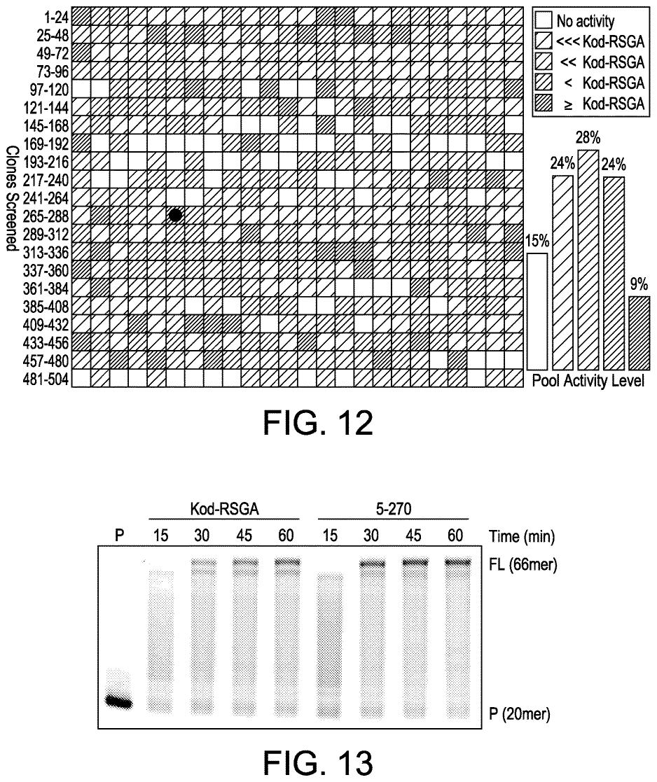

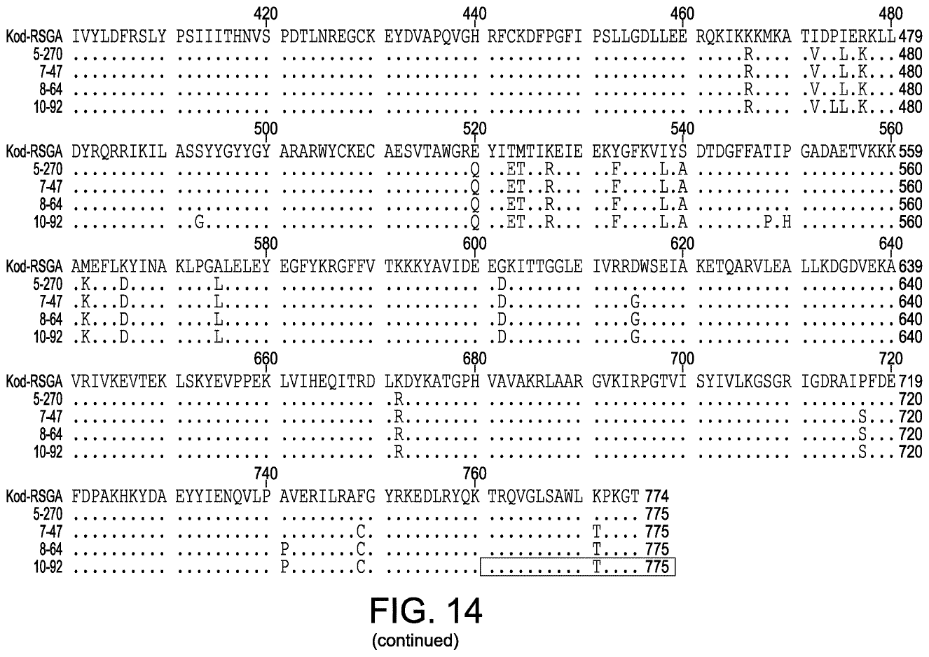

Iterative rounds of directed evolution were accomplished using the massively parallel screening technique of droplet-based optical polymerase sorting (DrOPS, Table 2)³⁶. DrOPS is a two-step microfluidic approach in which E. coli expressing different polymerase variants are individually encapsulated in uniform water-in-oil (w/o) droplets along with the reagents necessary to complete a polymerase activity assay (FIG. 1e), including chemically synthesized TNA triphosphates (tNTPs)³⁷. The droplet population (~10⁷) is collected, thermally lysed off-chip by prolonged heating at 95° C., and incubated at 55° C. to promote TNA synthesis on a self-priming DNA template. Polymerases that successfully copy the template into full-length TNA generate a fluorescent signal by disrupting a fluorophore-quencher pair located at the 5′ end of the DNA template. Fluorescent droplets are then recovered by passing the droplet population through a fluorescence-activated droplet sorting (FADS) device, which partitions droplets based on user-defined fluorescence values. Genes encoding active polymerases are recovered, amplified by PCR, reinserted into an expression vector, and transformed back into E. coli for another round of selection (FIG. 1f). After 5 rounds of neutral selection for TNA synthesis activity, a screen of 500 members identified clone 5-270 as a slightly more efficient TNA polymerase (~2-fold) than Kod-RSGA (FIG. 11-14)⁷. The sequence of clone 5-270 differs from its closest natural homolog (FIG. 15), Kod DNA polymerase, by 40 amino acid mutations and 1 Leu insertion located on α-helix 12 of the palm subdomain (FIG. 2a).

TABLE 2
Summary of droplet information and incubation conditions

| | | | Fluorescent | | | | |
|---|---|---|---|---|---|---|---|
| | | Top | Threshold | Incubation | Droplets | Droplets | Colonies |
| Round | Library | Variant | (afu) | 55° C. (h) | Detected | Sorted | Screened |
| 1 | Recomb. | — | 60 | 18 | 36,680,319 | 20,079 | — |
| 2 | Recomb. | — | 50 | 14 | 27,145,396 | 170,219 | — |
| 3 | Recomb. | — | 50 | 10 | 27,121,065 | 158,867 | — |
| 4 | Recomb. | — | 50 | 3 | 32,852,979 | 13,588 | — |
| 5 | Recomb. | 5-270 | 50 | 1 | 33,678,095 | 198,421 | 500 |
| 6 | epPCR: | — | 50 | 18 | 11,905,802 | 121,520 | 100 |
| | Thumb | | | | | | |
| 7 | epPCR: | 7-47 | 50 | 12 | 13,879,733 | 37,650 | 100 |
| | Thumb | | | | | | |
| 8 | epPCR: | 8.64 | 50 | 5 | 17,863,171 | 1,140 | 100 |
| | Thumb | | | | | | |
| 9 | epPCR: | 8.64 | 50 | 5 | 11,480,842 | 22,534 | 100 |
| | Thumb | | | | | | |
| 10 | epPCR: | 10-92 | 50 | 5 | 16,515,310 | 5,691 | 100 |
| | Palm, | | | | | | |
| | Finger | | | | | | |

Abbreviations: Homologous Recombination (Recomb.), error prone PCR (epPCR)

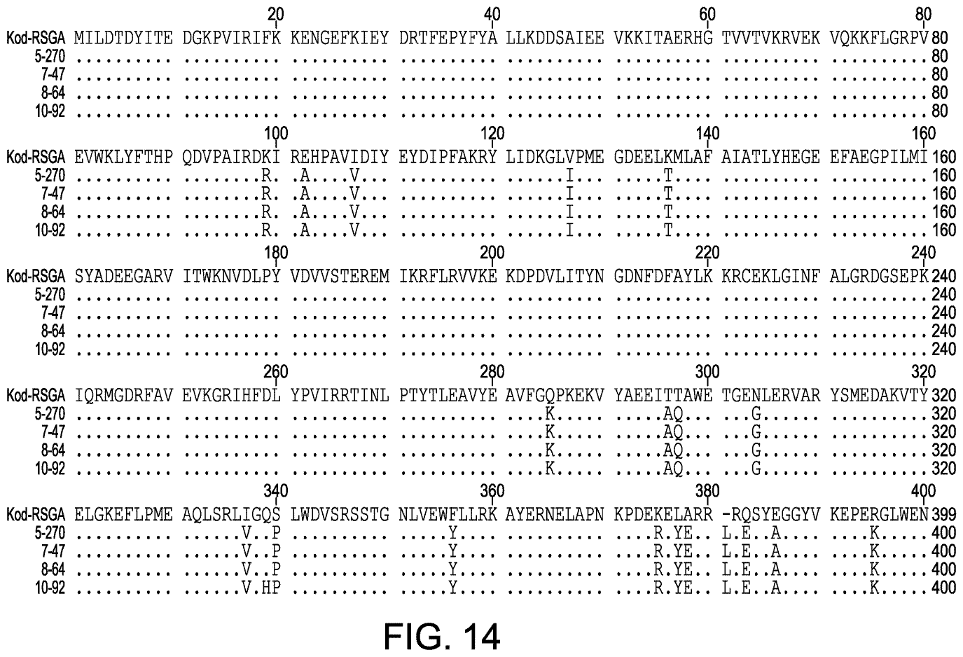

To identify variants with improved TNA synthesis activity, we performed a directed evolution campaign that began by intentionally mutating the thumb subdomain of clone 5-270 and carrying the library through two additional rounds of droplet sorting. Screening 100 clones from round 7 identified clone 7-47 as an improved variant with 4 mutations relative to 5-270 (FIG. 2a). Mutagenesis of the thumb subdomain of clone 7-47, followed by one round of selection, uncovered clone 8-64 as the top performing variant isolated from round 8. Clone 8-64 differs from 7-74 by a single point mutation (A741P) located in α-helix 21 in the thumb subdomain (FIG. 2a). Finally, we mutagenized the finger and palm subdomains of clone 8-64 and carried the new library through two more rounds of selection. Screening 100 clones from round 10 identified clone 10-92 as a highly efficient TNA polymerase with 5 additional mutations, two of which occur in the finger subdomain responsible for tNTP recognition (FIG. 2a,b). Comparative sequence analysis shows that 10-92 is a highly chimeric enzyme comprised of pieces from all four parent polymerases (FIG. 2c, FIG. 14), differing from natural Kod polymerase by 1 insertion and 50 amino acid mutations. These sequence changes occur throughout the protein, making 10-92 a highly divergent polymerase relative to other polymerases that have been previously developed for XNA synthesis¹.

### Biochemical Characterization

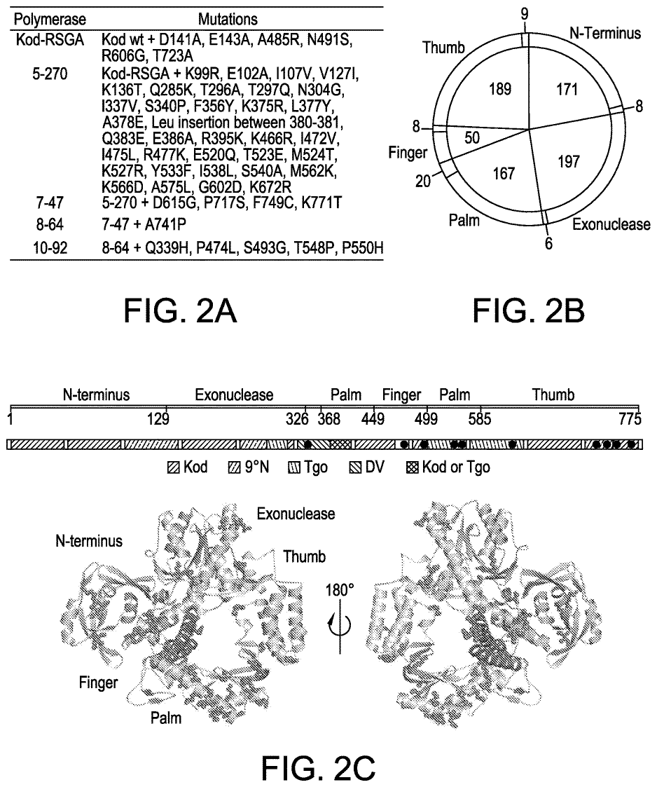

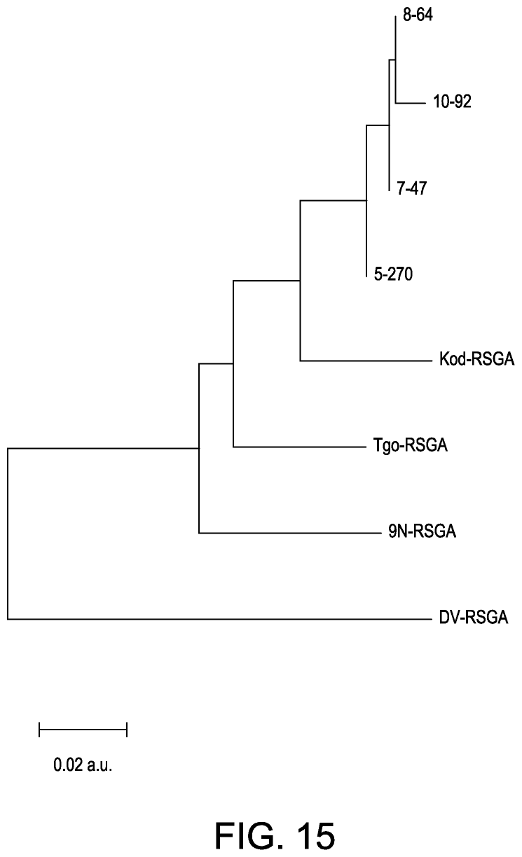

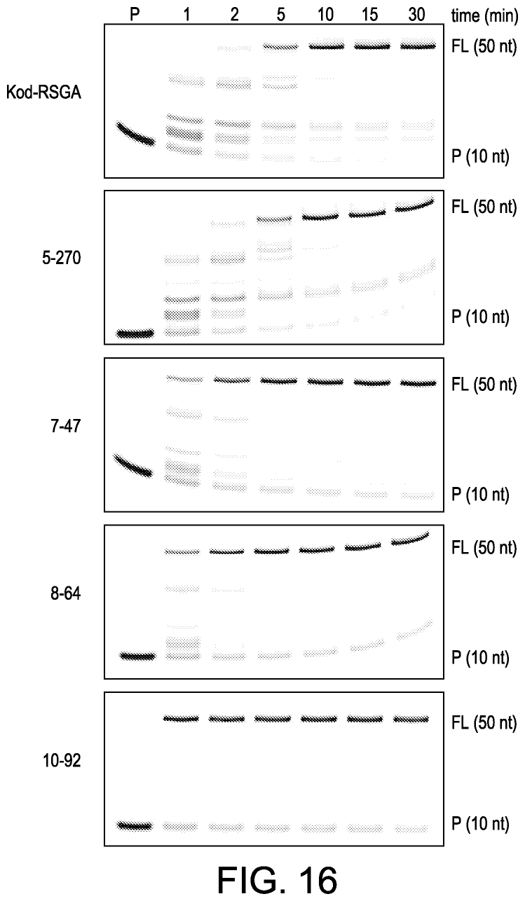

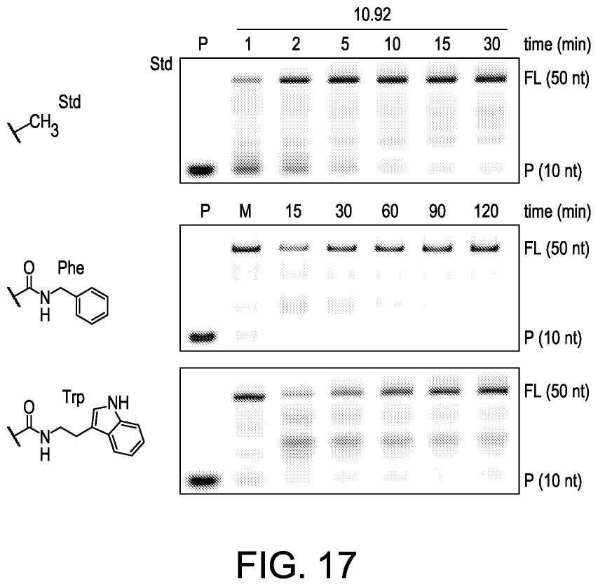

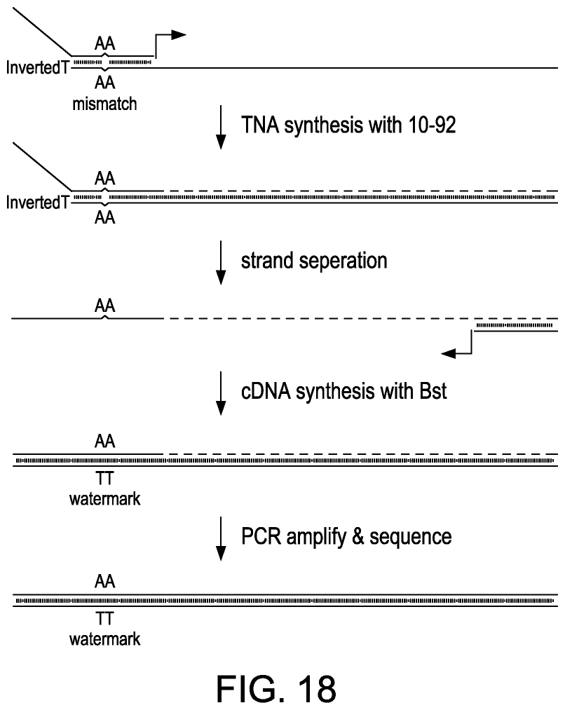

The enzymatic and biochemical properties of the 10-92 TNA polymerase were evaluated using standard polymerase assays. The results demonstrate that 10-92 is strikingly more efficient at TNA synthesis in standard magnesium buffer than any of its evolutionary progenitors (FIG. 3a,b, FIG. 16), capable of extending a DNA primer to full-length product with 40 nucleotide additions occurring in 45 seconds (FIG. 3c). The enhanced catalytic activity of 10-92 allows the enzyme to support TNA synthesis with reduced tNTP concentrations (4-fold) relative to Kod-RSGA, making it a more efficient enzyme for TNA synthesis. It also allows the enzyme to discriminate against dNTP substrates to a higher degree (4-fold) than Kod-RSGA (FIG. 3d,e), suggesting that 10-92 is on the path to becoming a more specialized enzyme for TNA synthesis. Consistent with its improved substrate recognition ability, the polymerase was found to be more accepting of base-modified tNTPs (FIG. 3f, FIG. 17), which are valuable substrates for functionally enhanced TNA aptamers, known as threomers²⁴. The fidelity of TNA synthesis was assessed by sequencing the product of a complete cycle of TNA replication (DNA→TNA→DNA). This assay, which measures the aggregate fidelity of replication using controls to eliminate PCR artifacts, reveals that 10-92 functions with >99% template copying fidelity, yielding only 3 mistakes out of 1000 nucleotide incorporation events (FIG. 18, FIG. 19). This is a notable improvement over previous generations, which suffered from G-G mismatches during TNA synthesis³⁸. Last, we confirmed that 10-92 maintains the extreme thermal stability of its natural homologs by showing that the TNA synthesis activity of 10-92 is undiminished after 6 hours of heating at 90° C. (FIG. 3g). We attribute the extreme thermal stability of 10-92 to the cell lysis step of the DrOPS protocol that requires the polymerase to survive a thermal challenge of 30 minutes at 95° C. Together, the combined properties of high catalytic activity, high fidelity, and high thermal stability make the 10-92 TNA polymerase an important tool in synthetic biology.

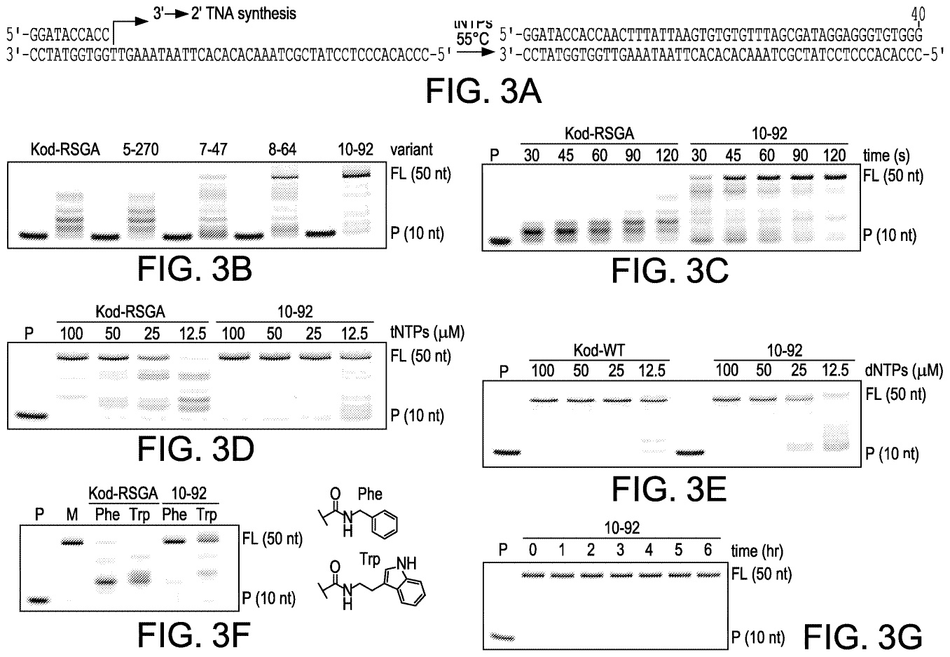

Intrigued by the catalytic efficiency of 10-92, we decided to measure the rate of TNA synthesis using the technique of polymerase kinetic profiling (PKPro)³⁹. In contrast to single-nucleotide primer-extension assays, which measure the efficiency at which a single nucleotide is added to the 3′ end of a DNA primer⁴⁰, PKPro provides a more holistic view of XNA synthesis by measuring the average rate of nucleotide incorporation across a template³⁹. As such, researchers are able to evaluate the entire catalytic cycle, which includes substrate recognition, nucleotide incorporation onto the growing strand, and continued extension from the modified position. For this assay, an unlabeled DNA primer-template duplex was extended with TNA by incubating the substrate with 10-92 and tNTPs at 55° C. The primer-extension reactions were stopped at designated times by plunging the reaction vessels into powdered dry ice. Once all the time points were collected, the reactions were thawed on ice in the presence of EvaGreen, previously identified as an optimal intercalating dye for TNA-DNA duplexes³⁹, and rates were evaluated by measuring the fluorescence signal of each sample. Background subtracted plots yield a rate of 54.4±1.0 nt/min for 10-92, as compared to 2.2±0.1 nt/min for Kod-RSGA (FIG. 4). To the best of our knowledge, this rate of XNA synthesis has not been witnessed previously by a laboratory-evolved XNA polymerase and is remarkable, given that TNA has one less atom in its backbone repeat unit than DNA¹⁸, which makes substrate recognition more challenging than DNA analogs with a standard 6-atom backbone repeating unit.

### Neutral Drift

Recognizing that the 10-92 TNA polymerase emerged from an evolutionary trajectory consisting of two mechanistically distinct steps, neutral selection achieved by homologous recombination and directed evolution facilitated by focused mutagenesis, we felt that this enzyme engineering example provided an interesting opportunity to evaluate the theory of neutral drift^{17,41}. It is generally accepted that neutral drift allows for improvements in protein function by enabling enzymes to traverse regions of the fitness landscape while avoiding the occurrence of inactive intermediates. In our case, the prolonged thermal lysis step favored the enrichment of stably folded proteins, while the TNA synthesis step ensured the inclusion of variants with desired functional activity. Following five rounds of neutral selection, the 41 mutations acquired by homologous recombination repositioned the polymerase in a region of the fitness landscape beyond the local sequence space of previous TNA polymerase libraries^{6,7}. Then, through the hill climbing exercise of directed evolution, the enzyme was able to acquire the precise sequence changes needed to adapt to a fitness maximum that allowed for improved catalytic activity.

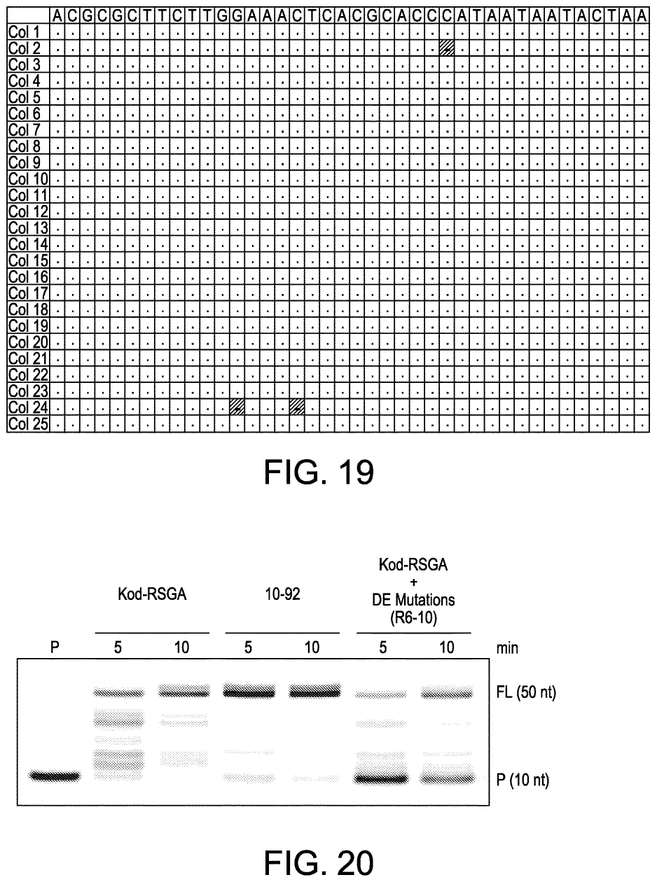

According to the theory of neutral drift, the beneficial mutations acquired by directed evolution should be specific to the protein sequence produced by neutral selection, since the parent sequences used to generate the starting library reside in a very different region of the fitness landscape⁴². To test this hypothesis, we evaluated the TNA synthesis activity of a Kod-RSGA variant that was engineered to contain the 10 beneficial mutations that were acquired during the directed evolution of 10-92 from the product of the neutral drift selection (TNA polymerase 5-270). Primer extension assays clearly show that the designed Kod-RSGA variant carrying the beneficial mutations functions with inferior activity relative to Kod-RSGA and 10-92 (FIG. 20). This observation supports the neutral drift theory of protein evolution by providing an example where neutral mutations give rise to a protein variant with heightened evolutionary potential, as illustrated in the epistatic nature of the 10 directed evolution mutations acquired during the evolution of the 10-92 TNA polymerase.

### Synthesis of Base-Modified TNA Aptamers

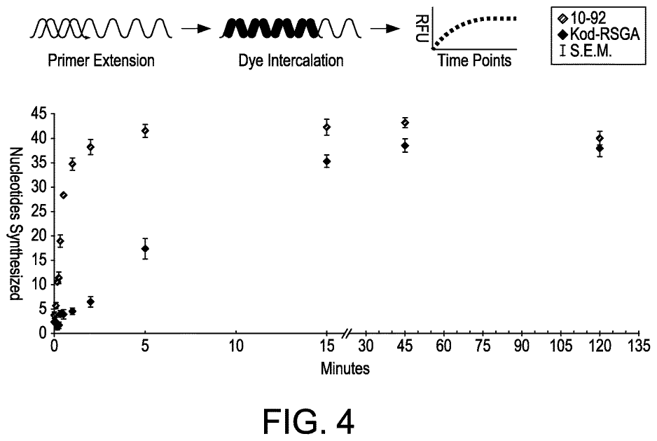

To illustrate the potential for 10-92 to function as an important new tool for synthetic biology, we compared the synthesis of three TNA aptamers using the TNA polymerases 10-92 and Kod-RSGA, which was the chemical basis of our starting library⁷. The aptamer sequences chosen for this study were selected from in vitro evolution experiments that were performed previously against the targets of HIV reverse-transcriptase (HIV-RT)⁴³, the receptor binding domain (RBD) of the S1 protein from SARS-CoV-2⁴⁴, and the full-length S1 protein from SARS-CoV-2²⁴. These aptamers comprise sequences of increasing synthetic difficulty as they transition from standard base chemistry for the HIV-RT aptamer to base-modified sequences carrying amide-linked phenylalanine and tryptophan side chains at the C-5 position of uracil bases for the RBD and S1 aptamers, respectively. Side-by-side primer extension studies reveal that 10-92 is strikingly more effective at synthesizing standard and base-modified TNA aptamers than Kod-RSGA by extending a 5′ labeled DNA primer annealed to the complementary DNA template (FIG. 5). Even the tryptophan chemistry, which historically has been a difficult chemotype to prepare by primer extension, was generated as a distinct full-length product with minor truncation bands as viewed by denaturing polyacrylamide gel electrophoresis (PAGE). The binding activity of the enzymatically generated and PAGE purified TNA aptamers was confirmed by biolayer interferometry (BLI) using TNA sequences carrying a 5′ biotin-labeled version of the DNA primer. Curve fitting of the resulting BLI sensorgrams reveals that the data conforms to a 1:1 binding model with calculated dissociation constants (K_{D}) in the low nanomolar range. As expected, TNA aptamers carrying the aromatic side chains function with slower off-rates than the standard base sequence (FIG. 5), making base-modified threomers possible reagents for diagnostic and therapeutic applications.

### Structural Analysis

The robust catalytic activity of the 10-92 TNA polymerase warrants a better understanding of the structural alterations needed to reprogram a natural DNA polymerase for strong XNA synthesis activity. Insights into this problem were obtained by solving the crystal structure of the closed ternary complex of 10-92 with the primer-template (P-T) duplex and incoming tNTP bound in the enzyme active site. Protein crystals grown in the presence of a chemically synthesized chain-terminating 2′-deoxy-α-L-threofuranosyl thymidine triphosphate (tTTP^{d})⁴⁵ and excess tATP trapped the enzyme in a catalytically active conformation. Diffraction-quality crystals were identified from a screen of ~900 conditions performed in a hanging-drop format using full-length and C-terminal truncated versions of the protein. The best crystal carried a 15 amino acid C-terminal deletion and resolved to a resolution limit of 2.8 Å. This structure was solved by molecular replacement using a previous TNA polymerase structure as the search model (Table 3)⁴⁶.

TABLE 3
Data collection and refinement statistics.

| | 10-92 | |
|---|---|---|
| Data Collection | | |
| Space group | P2₁2₁2₁ | |
| Cell Dimensions | | |
| a, b, c (Å) | 76.869, 99.525, 110.727 | |
| α, β, γ (°) | 90.0, 90.0, 90.0 | |
| Resolution (Å) | 45.39-2.73 | (2.828-2.73) |
| R_{merge} | 0.1773 | (0.97) |
| CC1/2 | 0.994 | (0.766) |
| I/σI | 11.36 | (2.24) |
| Completeness (%) | 99.03 | (98.20) |
| Redundancy | 6.6 | (6.8) |
| Refinement | | |
| Resolution (Å) | 2.73 | |
| No. reflections | 229979 | (2236) |
| R_{work}/R_{free} | 0.2088/0.2852 | (0.2902/0.3967) |

| | |
|---|---|
| No. atoms | 6724 |
| Protein/DNA | 6676 |
| tT/tATP | 48 |
| Solvent | — |
| B-factors | 45.01 |
| Protein/DNA | 44.26 |
| tT/tA | 43.52 |
| R.m.s deviations | |
| Bond lengths (Å) | 0.009 |
| Bond angles (°) | 1.12 |

* The statistics for the highest-resolution shell are shown in parenthesis.

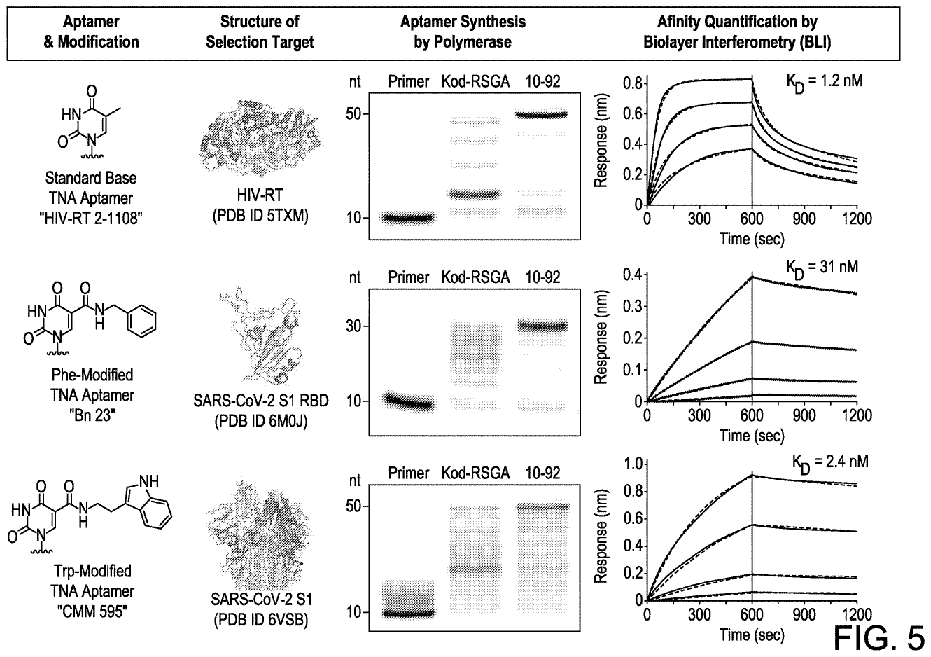

The 10-92 TNA polymerase adopts a disk-shaped architecture (FIG. 6) that is consistent with all known archaeal B-family DNA polymerases⁴⁷. Superposition of the new structure against the natural Kod DNA polymerase⁴⁸ reveals major conformational changes to the catalytic domain as well as small changes to the N-terminal (NTD) and exonuclease (Exo) domains (FIG. 6). These structural rearrangements, caused by the accumulation of neutral and beneficial mutations throughout the protein scaffold, primarily impact coordination of the enzyme to the P-T duplex (Table 4). Regions of the NTD domain shift by as much as 2 Å to interact with the unpaired region of the template strand, which is well resolved as it extends into the NTD (FIG. 6a). In the thumb subdomain, large positional rearrangements of the α-helical secondary structures (up to 7 Å) bring the thumb closer to the paired region of the DNA duplex (FIG. 6b), presumably improving the translocation efficiency of the enzyme. Another dramatic structural change occurs in the palm subdomain, which contains the catalytic aspartate residues required for chemical bond formation. Here, the Leu381 insertion, acquired by recombination with Pyrococcus DV, causes the α 12-helix to shift by 3 Å relative to its position in Kod DNA polymerase (FIG. 6c). This change to the protein sequence, along with neighboring mutations, is responsible for remodeling the enzyme active site and improving coordination to the DNA template.

TABLE 4
DNA-protein interactions in the ternary complex of 10-92.

| | Template | Primer | |
|---|---|---|---|
| | (5′-3′) | (2′-3′, 3′-5′) | |
| M244^{SBP}, G245^{B}, R247^{SBP}, N351^{SBP}, | C (T7) | | |
| K371^{SBP} | | | |
| R247^{SBP}, R266^{SBP}, R346^{SBP}, S347^{SBP}, | G (T8) | | |
| S348^{SBP}, N351^{SBP}, Y500^{SBP}, R502^{SBP} | | | |
| S348^{SBP}, T349^{SBP}, G350^{SBP}, N351^{SBP}, | T (T9) | tATP | D405^{P}, F406^{P}, S408^{P}, L409^{SP}, |
| I489^{B}, S492^{SBP}, G493^{B}, Y495^{SBP}, | | | Y410^{SP}, R461^{P}, R488^{P} S492^{S}, |
| G496^{SB}, Y497^{SBP}, Y500^{SBP} | | | D543^{SP}, T542^{S} |
| S384^{SB}, Y385^{SBP}, Y495^{SBP}, G496^{SB}, | A (T10) | tT^{d} | Y403^{P}, Y505^{P}, D541^{S,} K593^{S} |
| Y497^{SBP}, G499^{SBP}, Y500^{SBP}, A501^{BP} | | | |
| E383^{SBP}, S384^{SBP}, Y385^{SBP}, A386^{SB}, | C (T11) | G (P11) | D541^{SBP}, K593^{SBP}, Y595^{SBP}, |
| G387^{SB}, G388^{B}, Y495^{SBP} | | | T606^{SBP}, G607^{SBP}. G608^{SBP}, |
| | | | V612^{BP} |
| Y385^{SBP}, A386^{B}, G387^{SBP}, G388^{SBP}, | G (T12) | C (P10) | N269^{SBP}, K593^{SBP}, G608^{SB}, |
| V390^{SB}, K595^{SBP} | | | V612^{SB}, R613^{SBP}, R614^{SBP} |
| V390^{SBP}, K592^{SBP}, K593^{SBP} | C (T13) | G (P9) | R613^{SBP}, R614^{SBP}, H664^{SBP}, |
| | | | E665^{SBP}, Q666^{SBP}, T668^{SB} |
| E392^{SBP}, T591^{SBP}, K592^{SBP}, K594^{SBP}, | A (T14) | T (P8) | R613^{SBP}, E665^{SBP}, Q666^{SBP}, |
| R613^{SBP} | | | I667^{B}, T668^{SBP}, R669^{SBP}, |
| | | | Y674^{SBP}, H680^{SBP} |
| T591^{SBP}, K594^{SBP}, E610^{SBP}, R613^{SBP}, | G (T15) | C (P7) | T668^{SBP}, R669^{SBP}, D673^{SBP}, |
| W616^{SBP}, R744^{SBP} | | | Y674^{SBP}, K675^{SBP}, A676^{SBP}, |
| | | | H680^{SBP} |
| W616^{SBP}, N736^{SBP}, P740^{BP}, R744^{SBP} | T (T16) | A (P6) | R669^{SBP}, K675^{SBP}, A676^{SB} |
| G678^{SB}, P679^{SB}, I711^{SBP}, G712^{B}, | T (T17) | | |
| Y732^{SBP}, N736^{SBP}, Q737^{SBP} | | | |
| T677^{SBP}, G678^{SB}, P679^{B}, G709^{B}, | C (T18) | | |
| R710^{SBP}, I711^{BP}, G712^{BP}, D713^{SBP} | | | |
| R710^{SBP} | G (T19) | | |

Superscript denotes residue interactions with the sugar (S), base (B), and phosphate (P) moieties as identified by Mapiya²¹ and DNAproDB²². Mutations found in Kod-RSGA or 5-270 are underlined. TNA adducts shown in bold.

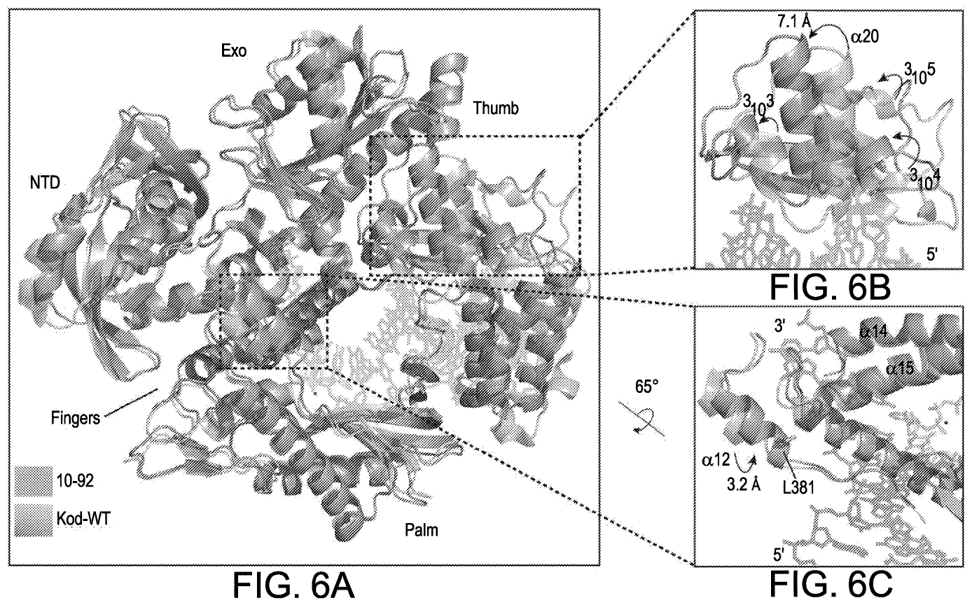

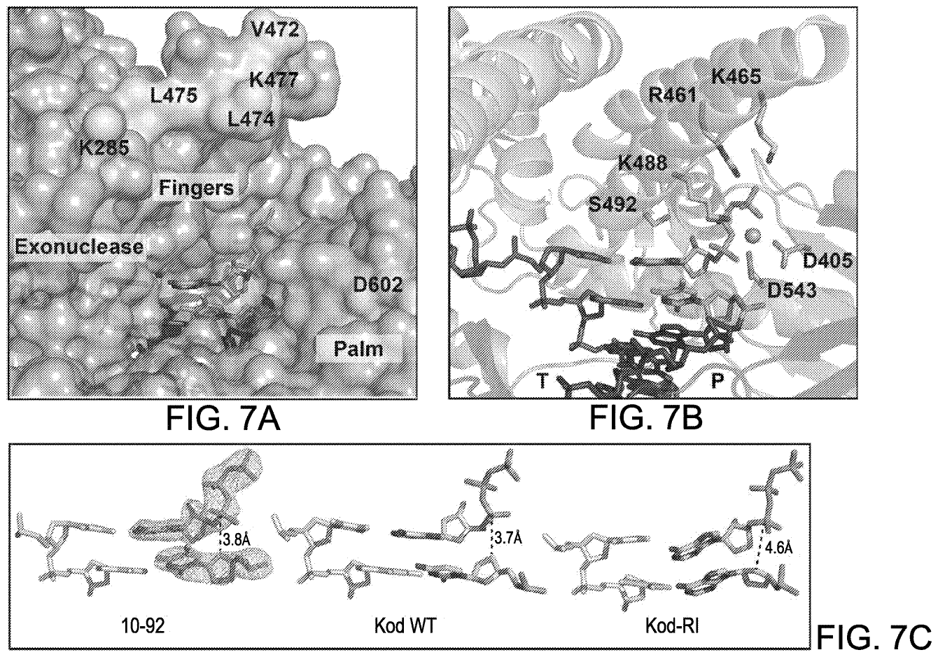

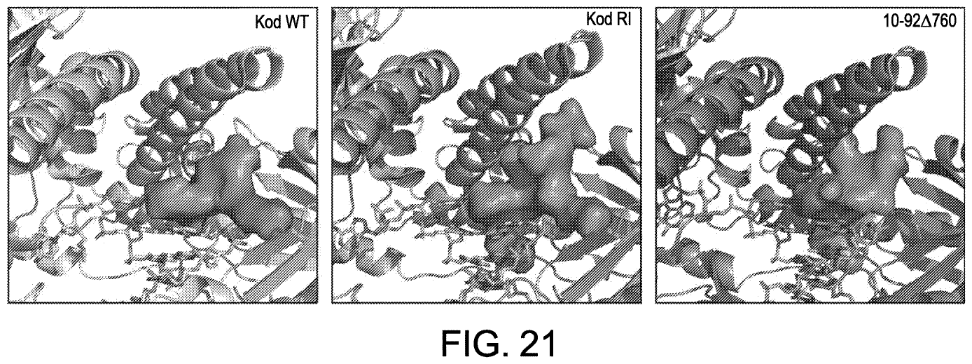

Close inspection of the crystal structure reveals the presence of a snuggly fit tATP substrate in the active site pocket formed by the palm and finger subdomains (FIG. 7a). Most of the mutations in this region of the polymerase reside in the O-helix of the finger subdomain, which is responsible for recognizing the incoming tNTP substrate. These mutations account for a smaller active site pocket as compared to a previously solved structure of an ancestral variant known as Kod-RI (FIG. 21)⁴⁶. The tATP substrate is stabilized by a series of Van der Waals interactions with the base and sugar moieties and electrostatic contacts to the phosphate tail, including the coordination of a tightly bound Mg²⁺ ion (FIG. 7b). Consistent with fast enzyme kinetics, the templating base (dT8) forms a favorable coplanar base pair with the tATP substrate (FIG. 7c, FIG. 8), thus correcting a suboptimal base pair geometry previously observed in the ancestral Kod-RI TNA polymerase structure⁴⁶. The improved base pairing ability of the incoming tATP substrate leads to a bond distance of 3.8 Å from the 2′ carbon atom of the threose sugar on the chain terminating primer to the α-phosphate of the incoming tATP substrate, which closely approximates the distance (3.7 Å) observed in the structure of natural Kod DNA polymerase⁴⁸. The remodeled active site represents an improvement over the ancestral Kod-RI polymerase, which exhibited a bond distance of 4.6 Å between equivalent atoms.

### DISCUSSION

The current study was motivated by our interest in understanding what level of sequence change is needed to transition a naturally occurring DNA polymerase into a highly efficient XNA polymerase capable of synthesizing artificial genetic polymers with backbone architectures that are not found in nature. Our earliest attempt at studying this problem revealed that natural DNA polymerases exhibit only limited levels of activity when TNA is used as either a nucleoside triphosphate or template⁴⁹⁻⁵¹, which is consistent with the large structural differences between DNA and TNA (3′→5′ versus 2′→3′ phosphodiester linkages)¹⁸ and the long evolutionary history that gave rise to DNA polymerases as essential enzymes for cellular life⁵². Subsequent efforts using the library diversification techniques of saturation mutagenesis and deep mutational scanning led to the discoveries of Kod-RI⁵³, Kod-RS⁶, and Kod-RSGA⁷ as first, second, and third generation TNA polymerases, respectively. Although these enzymes recognize TNA triphosphates with increasing efficiency, their catalytic rates are still much slower than natural DNA polymerases. A crystal structure of the ternary complex of Kod-RI indicates that this problem is due to the formation of a suboptimal geometry in the active site, whereby the incoming tNTP substrate struggles to pair with the templating base⁴⁶. As a consequence, many of our earlier TNA polymerases exhibited an increased propensity for G-G misincorporation during TNA synthesis⁵⁴. Although this problem can be overcome by replacing tGTP in the reaction mixture with its 7-deaza-guanine analog³⁸, this strategy limits the formation of certain folding topologies, like G-quadruplexes, that rely on Hoogsteen base pairing to adopt stably folded structures⁵⁵.

Our previous findings highlight the challenges faced by those attempting to engineer natural DNA polymerases for the ability to synthesize artificial genetic polymers unrelated to DNA and RNA. The limited number of studies performed thus far indicate that while it is relatively easy to broaden the substrate specificity of a natural DNA polymerase¹, converting such enzymes into highly specialized reagents that can recognize divergent substrates with high catalytic activity and template sequence fidelity remains a challenging problem. In the current study, we attempted to overcome this obstacle by implementing a neutral drift strategy that was intended to shift our protein to a region of the fitness landscape with heightened evolutionary potential. Then, through the sequence refinement process of directed evolution, identify variants within that region of sequence space that enable the enzyme to reach a new fitness maximum. The combined approach of neutral drift and directed evolution allowed us to identify 10-92 as a powerful new TNA polymerase that is capable of highly efficient and faithful primer-extension activity. This achievement represents a crucial advance in the development of TNA-based technologies, as protocols for solid phase TNA synthesis continue to lag behind those already established for DNA.

The crystal structure of the ternary complex offers valuable insight into the considerable catalytic activity achieved by the 10-92 TNA polymerase. Comparison of the 10-92 structure against its closest natural homolog, Kod DNA polymerase⁴⁸, reveals the degree of structural alteration imparted by the selected mutations. Regions of the catalytic domain, and to lesser extent the N-terminal and exonuclease domains, underwent major structural refinement with some secondary structural elements shifting by >7 Å relative to their position in Kod DNA polymerase. This finding suggests that the evolutionary distance required to transition natural DNA polymerases into highly specialized XNA enzymes may be larger than previously thought, as structural refinement of the active site likely requires major structural changes elsewhere in the protein architecture. Exploring this problem with other genetic systems will test current enzyme engineering strategies and offer a possible route to valuable reagents that can advance future biotechnology applications.

In summary, we describe the evolution and structure of a highly efficient TNA polymerase that can be used to expand the field of synthetic genetics. The enzyme functions with a catalytic rate of ~1 nt/sec and exhibits >99% template-sequence fidelity. The crystal structure of the enzyme trapped in a catalytically active conformation reveals major structural changes that greatly improved the ability for the incoming TNA triphosphate to pair with the templating base and form a covalent bond to the adjacent primer. Together, these results offer a possible path for evolving new examples of XNA polymerases that recognize distinct classes of genetic materials, including those for which such enzymes do not exist.

### REFERENCES

1 Nikoomanzar, A., et al. Quarterly Review of Biophysics 53, e8, (2020).
2 Tunyasuvunakool, K. et al. Nature 596, 590-596, (2021).
3 Wang, J. et al. Science 377, 387-394, (2022).
4 Chim, N., et al. Nat. Commun. 12, 2641, (2021).
5 Pinheiro, V. B. et al. Science 336, 341-344, (2012).
6 Nikoomanzar, A., et al. ACS Synth. Biol. 8, 1421-1429, (2019).
7 Nikoomanzar, A., et al. ACS Synthic Biology 9, 1873-1881, (2020).
8 Hoshino, H., et al. J. Am. Chem. Soc. 142, 21530-21537, (2020).
9 Medina, E., et al. ACS Synth. Biol. 10, 1429-1437, (2021).
10 Packer, M. S. & Liu, D. R. Nat. Rev. Genet. 16, 379-394, (2015).
11 Crameri, A., et al. Nature 391, 288-291, (1998).
12 Zhang, J. H., et al. Proc. Natl. Acad. Sci. U.S.A. 94, 4504-4509, (1997).
13 Chang, C. C. et al. Nat. Biotechnol. 17, 793-797, (1999).
14 Chiu, M. L., et al. Antibodies (Basel) 8, (2019).
15 Drummond, D. A., et al. Proc. Natl. Acad. Sci. U.S.A. 102, 5380-5385, (2005).
16 Khersonsky, O. & Tawfik, D. S. Annu. Rev. Biochem. 79, 471-505, (2010).
17 Romero, P. A. & Arnold, F. H. Nat Rev Mol Cell Biol 10, 866-876, (2009).
18 Schöning, K. U. et al. Science 290, 1347-1351, (2000).
19 Eschenmoser, A. Science 284, 2118-2124, (1999).
20 Culbertson, M. C. et al. Bioorg. Med. Chem. Lett. 26, 2418-2421, (2016).
21 Lee, E. M., et al. Nucleic Acids Res. 51, 9542-9551, (2023).
22 Yu, H., et al. Nat. Chem. 4, 183-187, (2012).
23 Dunn, M. R., et al. J. Am. Chem. Soc. 142, 7721-7724, (2020).
24 McCloskey, C. M. et al. ACS Synth. Biol. 10, 3190-3199, (2021).
25 Li, X., et al. Chem. Commun. 56, 14653-14656, (2020).
26 Wang, Y. et al. Nat. Chem. 14, 350-359, (2022).
27 Wang, Y. et al. J. Am. Chem. Soc. 143, 8154-8163, (2021).
28 Yang, K., et al. ACS Synth. Biol. 9, 2936-2942, (2020).
29 Wang, Y., et al. Nat. Chem. 13, 319-326, (2021).
30 Wang, F. et al. ACS Appl. Mater. Interfaces 11, 38510-38518, (2019).
31 Shigeo, M. et al. J. Am. Chem. Soc. 145, in press, (2023).
32 Gould, S. J. Wonderful life: the Burgess Shale and the nature of history. 1st edn, (W. W. Norton, 1989).
33 Hottin, A. & Marx, A. Acc. Chem. Res. 49, 418-427, (2016).
34 Yik, E. J., et al. Methods Enzymol., (2023).
35 Minshull, J. & Stemmer, W. P. Curr. Opin. Chem. Biol. 3, 284-290, (1999).
36 Vallejo, D., et al. ACS Synth. Biol. 8, 1430-1440, (2019).
37 Liao, J.-Y., et al. J. Am. Chem. Soc. 141, 13286-13289, (2019).
38 Mei, H. et al. J. Am. Chem. Soc. 140, 5706-5713, (2018).
39 Nikoomanzar, A., et al. Anal. Chem. 89, 12622-12625, (2017).
40 Goodman, M. F., et al. Critical Reviews of Biochemistry and Molecular Biology 28, 83-126, (1993).
41 Tokuriki, N. & Tawfik, D. S. Science 324, 203-207, (2009).
42 Kimura, M. The neutral theory of molecular evolution. (Cambridge University Press, 1983).
43 Kundu, N., et al. Biochemistry, in press., (2023).
44 Lozoya-Colinas, A., Yu, Y. & Chaput, J. C. J. Am. Chem. Soc., in press, (2023).
45 Bala, S. et al. J. Org. Chem. 83, 8840-8850, (2018).
46 Chim, N., et al. Nat. Commun. 8, 1810, (2017).
47 Rodriguez, A. C., et al. J. Mol. Biol. 299, 447-462, (2000).
48 Kropp, H. M., et al. PLOS One 12, e0188005, (2017).
49 Chaput, J. C. & Szostak, J. W. J. Am. Chem. Soc. 125, 9274-9275, (2003).
50 Chaput, J. C., et al. J. Am. Chem. Soc. 125, 856-857, (2003).
51 Horhota, A. et al. J. Am. Chem. Soc. 127, 7427-7434, (2005).
52 Steitz, T. A. J. Biol. Chem. 274, 17395-17398, (1999).
53 Dunn, M. R., et al. ACS Chem. Biol. 11, 1210-1219, (2016).
54 Dunn, M. R. et al. J. Am. Chem. Soc. 137, 4014-4017, (2015).
55 Platella, C., et al. Biochimica et Biophysica Acta (BBA)—General Subjects 1861, 1429-1447, (2017).

### Example 2: Methods and Materials

This Example provides details of the methods and materials used in the study described in Example 1 above.

### Reagents

DNA oligonucleotides and gBlocks were purchased from Integrated DNA Technologies (IDT, Coralville, Iowa). TNA triphosphates were obtained by chemical synthesis as described previously^{1,2}. ThermoPol buffer, Q5 DNA polymerase, NEB 5-alpha competent E. coli, Taq DNA polymerase, NdeI and NotI restriction enzymes, DpnI, and Gibson assembly were purchased from New England Biolabs (Ipswich, MA). XL1-Blue competent cells were purchased from Agilent (Santa Clara, CA). DNA clean up kit and ZymoPURE plasmid midiprep kit were purchased from Zymo (Irvine, CA). Express DNA Miniprep kit and Plasmid midiprep kit were purchased from Biomiga (San Diego, CA). Chemical reagents including dNTPs, manganese chloride and ammonium persulfate (APS) were purchased from Sigma Aldrich (St. Louis, Missouri). TOPO TA cloning kit, ethylenediaminetetraacetic acid (EDTA) were purchased from Thermofisher Scientific (Waltham, Massachusetts). SequalGel UreaGel 29:1 Denaturing Gel System was purchased from National Diagnostics (Atlanta, GA). Tetramethyl-ethylenediamine (TEMED) was purchased from Bio-Rad (Hercules, California). Heparin affinity columns were purchased from GE Healthcare (Little Chalfont, United Kingdom). Polydimethylsiloxane (PDMS) base and curing agent were purchased from Dow Corning (Midland, MI). SU-8 2025 photoresist was purchased from Fisher Scientific (Hampton, NH). Fluorinated oil HFE-7500 was purchased from 3M Novec (St Paul, MN), and Pico-Surf™ 1 surfactant, Pico-Glide™ 1, and Pico-Break™ 1 were all purchased from Dolomite Microfluidics (UK). EvaGreen® dye was purchased from Biotium (Fremont, CA). Clear V-bottom 96-well plates were purchased from Greiner (Monroe, NC). Plastic 1.5 mL micro-centrifuge tubes were purchased from Sigma-Aldrich (St. Louis, MO). Tygon tubing (EW-06419-01) was purchased from Cole-Parmer (Vernon Hills, IL). The SMC ITV0011-2UMS digital pressure regulator was purchased from Automation Distribution (Hatfield, PA). Microfluidic reagents HFE 7500, and Pico-Surf were purchased from 3M Novec (North Cordova, IL) and Sphere Fluidics, (Cambridge, UK), respectably. A 552 nm laser was purchased from Coherent OBIS LS (Santa Clara, CA). An apochromatic objective was purchased from Motic (Kowloon Bay, Hong Kong). A field-gated programmable array (FPGA, USB-7856R), was purchased from National Instruments (Austin, TX). The high-voltage amplifier was purchased from Trek Inc 2210 (Lockport, NY). Crystallization screens were purchased from Hampton Research (Aliso Viejo, CA), NeXtal Biosciences (Holland, OH), and Molecular Dimensions (Holland, OH). The Mosquito crystallization robot was purchased from SPT LabTech (Covina, CA). pET21 plasmid was purchased from Novagen Technology (Glendale, CA). The final crystallography reagents include HEPES, purchased from Sigma Aldrich (St. Louis, MO), polyethylene glycol 6000 and lithium chloride both purchased from Hampton research (Aliso Viejo, CA). All Sanger sequencing was performed by Genewiz (San Diego, CA)

### Design and Construction of Recombination Library

A custom homologous recombination library was designed based on the sequence alignment of four homologous hyperthermophilic family-B DNA polymerases isolated from archaeal species Thermococcus kodakaraensis (Kod, GenBank: KP682508.1), Thermococcus gorgonarius (Tgo, GenBank: KP682507.1), Pyrococcus species GB-D Deep Vent (DV, GenBank: KP682509.1), and Thermococcus sp. 9°N (9°N, GenBank: KP682506.1). Each gene was modified to contain the following amino acid mutations: E141A, D143A, A485R, N491S, R606G, and T723A. The gene encoding each enzyme was translated in silico, aligned, and sequence verified. Following in silico validation, 11 regions were identified as segments (termed cassettes) for subsequent homologous recombination by DNA shuffling. The boundaries between each cassette (7-10 amino acids) were modified by codon swapping to ensure perfect homology between each of the four parent genes. This was required for homologous recombination by overlap PCR. Finally, the genes were modified to include overlap regions with the backbone vector, as required for Gibson assembly. The genes were ordered as gBlocks (Table 5).

TABLE 5
DNA gBlocks
Oligonucleotide sequences ordered from IDT written in the 5′→3′ direction.

| Name | DNA Sequence (5′→3′) |
|---|---|
| Kod-RSGA_gene | ATGATCCTCGACACTGACTACATAACCGAGGATGGAAAGCCTGTCATAAGAATTTTCA |
| (SEQ ID NO: 4) | AGAAGGAAAACGGCGAGTTTAAGATTGAGTACGACCGGACTTTTGAACCCTACTTCTA |
| | CGCCCTCCTGAAGGACGATTCTGCCATTGAGGAAGTCAAGAAGATAACCGCCGAGAGG |
| | CACGGGACGGTTGTAACGGTTAAGCGGGTTGAAAAGGTTCAGAAGAAGTTCCTCGGGA |
| | GACCAGTTGAGGTCTGGAAACTCTACTTTACTCATCCGCAGGACGTCCCAGCGATAAG |
| | GGACAAGATACGAGAGCATCCAGCAGTTATTGACATCTACGAGTACGACATACCCTTC |
| | GCCAAGCGCTACCTCATAGACAAGGGATTAGTGCCAATGGAAGGCGACGAGGAGCTGA |
| | AAATGCTCGCCTTCGCGATTGCGACTCTCTACCATGAGGGCGAGGAGTTCGCCGAGGG |
| | GCCAATCCTTATGATAAGCTACGCCGACGAGGAAGGGGCCAGGGTGATAACTTGGAAG |
| | AACGTGGATCTCCCCTACGTTGACGTCGTCTCGACGGAGAGGGAGATGATAAAGCGCT |
| | TCCTCCGTGTTGTGAAGGAGAAAGACCCGGACGTTCTCATAACCTACAACGGCGACAA |
| | CTTCGACTTCGCCTATCTGAAAAAGCGCTGTGAAAAGCTCGGAATAAACTTCGCCCTC |
| | GGAAGGGATGGAAGCGAGCCGAAGATTCAGAGGATGGGCGACAGGTTTGCCGTCGAAG |
| | TGAAGGGACGGATACACTTCGATCTCTATCCTGTGATAAGACGGACGATAAACCTGCC |
| | CACATACACGCTTGAGGCCGTTTATGAAGCCGTCTTCGGTCAGCCGAAGGAGAAGGTT |
| | TACGCTGAGGAAATAACCACAGCCTGGGAAACCGGCGAGAACCTTGAGAGAGTCGCCC |
| | GCTACTCGATGGAAGATGCGAAGGTCACATACGAGCTTGGGAAGGAGTTCCTTCCGAT |
| | GGAGGCCCAGCTTTCTCGCTTAATCGGCCAGTCCCTCTGGGACGTCTCCCGCTCCAGC |
| | ACTGGCAACCTCGTTGAGTGGTTCCTCCTCAGGAAGGCCTATGAGAGGAATGAGCTGG |
| | CCCCGAACAAGCCCGATGAAAAGGAGCTGGCCAGAAGACGGCAGAGCTATGAAGGAGG |
| | CTATGTAAAAGAGCCCGAGAGAGGGTTGTGGGAGAACATAGTGTACCTAGATTTTAGA |
| | TCCCTGTACCCCTCAATCATCATCACCCACAACGTCTCGCCGGATACGCTCAACAGAG |
| | AAGGATGCAAGGAATATGACGTTGCCCCACAGGTCGGCCACCGCTTCTGCAAGGACTT |
| | CCCAGGATTTATCCCGAGCCTGCTTGGAGACCTCCTAGAGGAGAGGCAGAAGATAAAG |
| | AAGAAGATGAAGGCCACGATTGACCCGATCGAGAGGAAGCTCCTCGATTACAGGCAGA |
| | GGCGTATCAAGATCCTGGCAAGCAGCTACTACGGTTACTACGGCTATGCAAGGGCGCG |
| | CTGGTACTGCAAGGAGTGTGCAGAGAGCGTAACGGCCTGGGGAAGGGAGTACATAACG |
| | ATGACCATCAAGGAGATAGAGGAAAAGTACGGCTTTAAGGTAATCTACAGCGACACCG |
| | ACGGATTTTTTGCCACAATACCTGGAGCCGATGCTGAAACCGTCAAAAAGAAGGCTAT |
| | GGAGTTCCTCAAGTATATCAACGCCAAACTTCCGGGCGCGCTTGAGCTCGAGTACGAG |
| | GGCTTCTACAAACGCGGCTTCTTCGTCACGAAGAAGAAGTATGCGGTGATAGACGAGG |
| | AAGGCAAGATAACAACGGGTGGACTTGAGATTGTGAGGCGTGACTGGAGCGAGATAGC |
| | GAAAGAGACGCAGGCGAGGGTTCTTGAAGCTTTGCTAAAGGACGGTGACGTCGAGAAG |
| | GCCGTGAGGATAGTCAAAGAAGTTACCGAAAAGCTGAGCAAGTACGAGGTTCCGCCGG |
| | AGAAGCTGGTGATCCACGAGCAGATAACGAGGGATTTAAAGGACTACAAGGCAACCGG |
| | TCCCCACGTTGCCGTTGCCAAGAGGTTGGCCGCGAGAGGAGTCAAAATACGCCCTGGA |
| | ACGGTGATAAGCTACATCGTGCTCAAGGGCTCTGGGAGGATAGGCGACAGGGCGATAC |
| | CGTTCGACGAGTTCGACCCGGCGAAGCACAAGTACGACGCCGAGTACTACATTGAGAA |
| | CCAGGTTCTCCCAGCCGTTGAGAGAATTCTGAGAGCCTTCGGTTACCGCAAGGAAGAC |
| | CTGCGCTACCAGAAGACGAGACAGGTTGGTTTGAGTGCTTGGCTGAAGCCGAAGGGAA |
| | CTTGA |
| Tgo_RSGA_ | AGCGGCCTGGTGCCGCGCGGCAGCATGATCCTCGACACTGACTACATAACCGAGGATG |
| gBlock | GAAAGCCTGTCATAAGAATTTTCAAGAAGGAAAACGGCGAGTTTAAGATTGACTACGA |
| (SEQ ID NO: 5) | CCGGAACTTTGAACCCTACATCTACGCCCTCCTGAAGGACGATTCTGCCATTGAGGAC |
| | GTCAAGAAGATAACCGCCGAGAGGCACGGGACGACCGTAAGGGTTGTCCGGGCCGAAA |
| | AGGTTAAGAAGAAGTTCCTCGGGAGACCAATAGAGGTCTGGAAACTCTACTTTACTCA |
| | TCCGCAGGACGTCCCAGCGATAAGGGACAAGATAAAGGAGCATCCAGCAGTTGTGGAC |
| | ATCTACGAGTACGACATACCCTTCGCCAAGCGCTACCTCATAGACAAGGGATTAATCC |
| | CAATGGAAGGCGACGAGGAGCTGAAAATGCTCGCCTTCGCGATTGCGACTCTCTACCA |
| | TGAGGGCGAGGAGTTCGCCGAGGGGCCAATCCTTATGATAAGCTACGCCGACGAGGAA |
| | GGGGCCAGGGTGATAACTTGGAAGAACATCGATCTCCCCTACGTTGACGTCGTCTCGA |
| | CGGAGAAGGAGATGATAAAGCGCTTCCTCAAGGTTGTGAAGGAGAAAGACCCGGACGT |
| | TCTCATAACCTACAACGGCGACAACTTCGACTTCGCCTATCTGAAAAAGCGCTCCGAA |
| | AAGCTCGGAGTCAAGTTCATCCTCGGAAGGGAAGGAAGCGAGCCGAAGATTCAGAGGA |
| | TGGGCGACAGGTTTGCCGTCGAAGTGAAGGGACGGATACACTTCGATCTCTATCCTGT |
| | GATAAGACGGACGATAAACCTGCCCACATACACGCTTGAGGCCGTTTATGAAGCCATC |
| | TTCGGTCAGCCGAAGGAGAAGGTTTACGCTGAGGAAATAGCGCAGGCCTGGGAAACCG |
| | GCGAGGGACTTGAGAGAGTCGCCCGCTACTCGATGGAAGATGCGAAGGTCACATACGA |
| | GCTTGGGAAGGAGTTCTTCCCGATGGAGGCCCAGCTTTCTCGCTTAGTAGGCCAGTCC |
| | CTCTGGGACGTCTCCCGCTCCAGCACTGGCAACCTCGTTGAGTGGTTCCTCCTCAGGA |
| | AGGCCTATGAGAGGAATGAGCTGGCCCCGAACAAGCCCGATGAAAGGGAGCTGGCCAG |
| | AAGACGGGAGAGCTATGCGGGAGGCTATGTAAAAGAGCCCGAGAGAGGGTTGTGGGAG |
| | AACATAGTGTACCTAGATTTTAGATCCCTGTACCCCTCAATCATCATCACCCACAACG |
| | TCTCGCCGGATACGCTCAACAGAGAAGGATGCGAGGAATATGACGTTGCCCCACAGGT |
| | CGGCCACAAGTTCTGCAAGGACTTCCCAGGATTTATCCCGAGCCTGCTTGGAGACCTC |
| | CTAGAGGAGAGGCAGAAGGTAAAGAAGAAGATGAAGGCCACGATTGACCCGATCGAGA |
| | AGAAGCTCCTCGATTACAGGCAGAGGCGTATCAAGATCCTGGCAAGCAGCTTCTACGG |
| | TTACTACGGCTATGCAAAGGCGCGCTGGTACTGCAAGGAGTGTGCAGAGAGCGTAACG |
| | GCCTGGGGAAGGCAGTACATAGAGACCACCATCAGGGAGATAGAGGAAAAGTTTGGCT |
| | TTAAGGTACTCTACGCGGACACCGACGGATTTTTTGCCACAATACCTGGAGCCGATGC |
| | TGAAACCGTCAAAAAGAAGGCTAAGGAGTTCCTCGACTATATCAACGCCAAACTTCCG |
| | GGCCTGCTTGAGCTCGAGTACGAGGGCTTCTACAAACGCGGCTTCTTCGTCACGAAGA |
| | AGAAGTATGCGGTGATAGACGAGGAAGACAAGATAACAACGGGTGGACTTGAGATTGT |
| | GAGGCGTGACTGGAGCGAGATAGCGAAAGAGACGCAGGCGAGGGTTCTTGAAGCTATA |
| | CTAAAGCACGGTGACGTCGAGGAAGCCGTGAGGATAGTCAAAGAAGTTACCGAAAAGC |
| | TGAGCAAGTACGAGGTTCCGCCGGAGAAGCTGGTGATCTACGAGCAGATAACGAGGGA |
| | TTTAAAGGACTACAAGGCAACCGGTCCCCACGTTGCCGTTGCCAAGAGGTTGGCCGCG |
| | AGAGGAATAAAAATACGCCCTGGAACGGTGATAAGCTACATCGTGCTCAAGGGCTCTG |
| | GGAGGATAGGCGACAGGGCGATACCGTTCGACGAGTTCGACCCGGCGAAGCACAAGTA |
| | CGACGCCGAGTACTACATTGAGAACCAGGTTCTCCCAGCCGTTGAGAGAATTCTGAGA |
| | GCCTTCGGTTACCGCAAGGAAGACCTGCGCTACCAGAAGACGAGACAGGTTGGTTTGG |
| | GGGCTTGGCTGAAGCCGAAGACATGAGTCGACCTGCAGCCAAGCTTA |
| DV_RSGA_ | AGCGGCCTGGTGCCGCGCGGCAGCATGATCCTCGACGCTGACTACATAACCGAGGATG |
| gBlock | GAAAGCCTATTATAAGAATTTTCAAGAAGGAAAACGGCGAGTTTAAGGTTGAGTACGA |
| (SEQ ID NO: 6) | CCGGAACTTTAGACCCTACATTTACGCCCTCCTGAAGGACGATTCTCAGATTGATGAA |
| | GTCAGGAAGATAACCGCCGAGAGGCACGGGAAGATAGTAAGAATTATAGATGCCGAAA |
| | AGGTTAGGAAGAAGTTCCTCGGGAGACCAATTGAGGTCTGGAGGCTCTACTTTGAACA |
| | TCCGCAGGACGTCCCAGCGATAAGGGACAAGATACGAGAGCATTCCGCAGTTATTGAC |
| | ATCTTTGAGTACGACATACCCTTCGCCAAGCGCTACCTCATAGACAAGGGATTAATTC |
| | CAATGGAAGGCGACGAGGAGCTGAAATTGCTCGCCTTCGCGATTGCGACTCTCTACCA |
| | TGAGGGCGAGGAGTTCGCCAAGGGGCCAATCATAATGATAAGCTACGCCGACGAGGAA |
| | GAAGCCAAAGTGATAACTTGGAAGAAGATCGATCTCCCCTACGTTGAGGTCGTCTCGA |
| | GCGAGAGGGAGATGATAAAGCGCTTCCTCAAGGTTATAAGGGAGAAAGACCCGGACGT |
| | TATAATAACCTACAACGGCGACTCTTTCGACCTTCCCTATCTGGTTAAGCGCGCCGAA |
| | AAGCTCGGAATAAAGCTACCCCTCGGAAGGGATGGAAGCGAGCCGAAGATGCAGAGGC |
| | TTGGCGACATGACAGCCGTCGAAATAAAGGGACGGATACACTTCGATCTCTATCACGT |
| | GATAAGACGGACGATAAACCTGCCCACATACACGCTTGAGGCCGTTTATGAAGCCATC |
| | TTCGGTAAGCCGAAGGAGAAGGTTTACGCTCACGAAATAGCTGAGGCCTGGGAAACCG |
| | GCAAGGGACTTGAGAGAGTCGCCAAGTACTCGATGGAAGATGCGAAGGTCACATACGA |
| | GCTTGGGAGGGAGTTCTTCCCGATGGAGGCCCAGCTTTCTCGCTTAGTCGGCCAGCCC |
| | CTCTGGGACGTCTCCCGCTCCAGCACTGGCAACCTCGTTGAGTGGTACCTCCTCAGGA |
| | AGGCCTATGAGAGGAATGAGCTGGCCCCGAACAAGCCCGATGAAAGGGAGTACGAGAG |
| | AAGGCTACGGGAGAGCTATGCTGGAGGCTATGTAAAAGAGCCCGAGAAAGGGTTGTGG |
| | GAGGGGTTAGTGTCCCTAGATTTTAGATCCCTGTACCCCTCAATCATCATCACCCACA |
| | ACGTCTCGCCGGATACGCTCAACAGAGAAGGATGCAGGGAATATGACGTTGCCCCAGA |
| | GGTCGGCCACAAGTTCTGCAAGGACTTCCCAGGATTTATCCCGAGCCTGCTTAAGAGG |
| | CTCCTAGATGAGAGGCAGGAAATAAAGAGGAAGATGAAGGCCTCTAAAGACCCGATCG |
| | AGAAGAAGATGCTCGATTACAGGCAGAGGCGTATCAAGATCCTGGCAAGCAGCTACTA |
| | CGGTTACTACGGCTATGCAAAAGCGCGCTGGTACTGCAAGGAGTGTGCAGAGAGCGTA |
| | ACGGCCTGGGGAAGGGAGTACATAGAGTTCGTAAGGAAGGAGCTGGAGGAAAAGTTCG |
| | GCTTTAAGGTATTATACATAGACACCGACGGACTCTACGCCACAATACCTGGAGCCAA |
| | ACCCGAAGAGATAAAAAAGAAGGCTCTAGAGTTCGTAGATTATATCAACGCCAAACTT |
| | CCGGGCCTGCTTGAGCTCGAGTACGAGGGCTTCTACGTGCGCGGCTTCTTCGTCACGA |
| | AGAAGAAGTATGCGTTGATAGACGAGGAAGGCAAGATAATCACGGGTGGACTTGAGAT |
| | TGTGAGGCGTGACTGGAGCGAGATAGCGAAAGAGACGCAGGCGAAAGTTCTTGAAGCT |
| | ATCCTAAAGCATGGTAACGTCGAGGAGGCCGTGAAGATAGTCAAAGAAGTTACCGAAA |
| | AGCTGAGCAAGTACGAGATACCGCCGGAGAAGCTGGTGATCTACGAGCAGATAACGAG |
| | GCCCTTACACGAGTACAAGGCAATAGGTCCCCACGTTGCCGTTGCCAAGAGGTTGGCC |
| | GCGAGAGGAGTCAAAGTGCGCCCTGGAATGGTGATAGGGTACATCGTGCTCAGGGGCG |
| | ACGGGCCAATAAGCAAGAGGGCGATACTTGCAGAGGAGTTCGACCTCGCGAAGCACAA |
| | GTACGACGCCGAGTACTACATTGAGAACCAGGTTCTCCCAGCCGTTCTTAGAATTCTG |
| | GAGGCCTTCGGTTACCGCAAGGAAGACCTGCGCTGGCAGAAGACGAAACAGACAGGTT |
| | TGACGGCTTGGCTGAACATCAAGAAGAAGTGAGTCGACCTGCAGCCAAGCTTA |
| 9º N_RSGA_ | AGCGGCCTGGTGCCGCGCGGCAGCATGATCCTCGACACTGACTACATAACCGAGAACG |
| gBlock | GAAAGCCTGTCATAAGAGTGTTCAAGAAGGAAAACGGCGAGTTTAAGATTGAGTACGA |
| (SEQ ID NO: 7) | CCGGACTTTTGAACCCTACTICTACGCCCTCCTGAAGGACGATTCTGCCATTGAGGAT |
| | GTCAAGAAGGTGACCGCCAAAAGGCACGGGACGGTTGTAAAAGTTAAGCGGGCGGAAA |
| | AGGTTCAGAAGAAGTTCCTCGGGAGACCAATTGAGGTCTGGAAACTCTACTTTAACCA |
| | TCCGCAGGACGTCCCAGCGATAAGGGACCGTATACGAGCGCATCCAGCAGTIGTGGAC |
| | ATCTACGAGTACGACATACCCTTCGCCAAGCGCTACCTCATAGACAAGGGATTAATTC |
| | CAATGGAAGGCGACGAGGAGCTGACCATGCTCGCCTTCGCGATTGCGACTCTCTACCA |
| | TGAGGGCGAGGAGTTCGGCACCGGGCCAATCCTTATGATAAGCTACGCCGACGGCAGC |
| | GAAGCCAGGGTGATAACTTGGAAGAAAATCGATCTCCCCTACGTTGACGTCGTCTCGA |
| | CGGAGAAAGAGATGATAAAGCGCTTCCTCCGTGTTGTGCGTGAGAAAGACCCGGACGT |
| | TCTCATAACCTACAACGGCGACAACTTCGACTTCGCCTATCTGAAAAAGCGCTGTGAA |
| | GAACTCGGAATAAAATTCACCCTCGGAAGGGATGGAAGCGAGCCGAAGATTCAGAGGA |
| | TGGGCGACAGGTTTGCCGTCGAAGTGAAGGGACGGATACACTTCGATCTCTATCCTGT |
| | GATAAGACGGACGATAAACCTGCCCACATACACGCTTGAGGCCGTTTATGAAGCCGTC |
| | TTCGGTAAACCGAAGGAGAAGGTTTACGCTGAGGAAATAGCGCAGGCCTGGGAAAGCG |
| | GCGAGGGCCTTGAGAGAGTCGCCCGCTACTCGATGGAAGATGCGAAGGTCACATACGA |
| | GCTTGGGCGTGAGTTCTTCCCGATGGAGGCCCAGCTTTCTCGCTTAATCGGCCAGTCC |
| | CTCTGGGACGTCTCCCGCTCCAGCACTGGCAACCTCGTTGAGTGGTTCCTCCTCAGGA |
| | AGGCCTATAAAAGGAATGAGCTGGCCCCGAACAAGCCCGATGAACGTGAGCTGGCCAG |
| | ACGTCGTGGCGGTTATGCGGGAGGCTATGTAAAAGAGCCCGAGAGAGGGTTGTGGGAT |
| | AACATAGTGTACCTAGATTTTAGATCCCTGTACCCCTCAATCATCATCACCCACAACG |
| | TCTCGCCGGATACGCTCAACAGAGAAGGATGCAAGGAATATGACGTTGCCCCAGAAGT |
| | CGGCCACAAATTCTGCAAGGACTTCCCAGGATTTATCCCGAGCCTGCTTGGAGACCTC |
| | CTAGAGGAGAGGCAGAAGATAAAGCGCAAGATGAAGGCCACGGTTGACCCGCTGGAGA |
| | AAAAGCTCCTCGATTACAGGCAGAGGCGTATCAAGATCCTGGCAAGCAGCTTCTACGG |
| | TTACTACGGCTATGCAAAAGCGCGCTGGTACTGCAAGGAGTGTGCAGAGAGCGTAACG |
| | GCCTGGGGAAGGGAGTACATAGAAATGGTGATCCGCGAGCTGGAGGAAAAGTTCGGCT |
| | TTAAGGTACTGTACGCGGACACCGACGGACTGCATGCCACAATACCTGGAGCCGATGC |
| | TGAAACCGTCAAAAAGAAGGCTAAAGAGTTCCTCAAGTATATCAACCCGAAACTTCCG |
| | GGCCTGCTTGAGCTCGAGTACGAGGGCTTCTACGTGCGCGGCTTCTTCGTCACGAAGA |
| | AGAAGTATGCGGTGATAGACGAGGAAGGCAAGATAACAACGGGTGGACTTGAGATTGT |
| | GAGGCGTGACTGGAGCGAGATAGCGAAAGAGACGCAGGCGAGGGTTCTTGAAGCTATT |
| | CTAAAGCATGGTGACGTCGAGGAAGCCGTGAGGATAGTCAAAGAAGTTACCGAAAAGC |
| | TGAGCAAGTACGAGGTTCCGCCGGAGAAGCTGGTGATCCACGAGCAGATAACGAGGGA |
| | TTTACGTGACTACAAGGCAACCGGTCCCCACGTTGCCGTTGCCAAGAGGTTGGCCGCG |
| | AGAGGAGTCAAAATACGCCCTGGAACGGTGATAAGCTACATCGTGCTCAAGGGCTCTG |
| | GGAGGATAGGCGACAGGGCGATACCGGCGGACGAGTTCGACCCGGCGAAGCACCGTTA |
| | CGACGCCGAGTACTACATTGAGAACCAGGTTCTCCCAGCCGTTGAGAGAATTCTGAAA |
| | GCCTTCGGTTACCGCAAGGAAGACCTGCGCTACCAGAAGACGAAACAGGTTGGTTTGG |
| | GCGCTTGGCTGAAGGTTAAGGGAAAATGAGTCGACCTGCAGCCAAGCTTA |
| Kod-RSGA + DE | GAGTTCCTTCCGATGGAGGCCCAGCTTTCTCGCTTAATCGGCCATTCCCTCTGGGACG |
| mutations (R6-10) | TCTCCCGCTCCAGCACTGGCAACCTCGTTGAGTGGTTCCTCCTCAGGAAGGCCTATGA |
| (SEQ ID NO: 8) | GAGGAATGAGCTGGCCCCGAACAAGCCCGATGAAAAGGAGCTGGCCAGAAGACGGCAG |
| | AGCTATGAAGGAGGCTATGTAAAAGAGCCCGAGAGAGGGTTGTGGGAGAACATAGTGT |
| | ACCTAGATTTTAGATCCCTGTACCCCTCAATCATCATCACCCACAACGTCTCGCCGGA |
| | TACGCTCAACAGAGAAGGATGCAAGGAATATGACGTTGCCCCACAGGTCGGCCACCGC |
| | TTCTGCAAGGACTTCCCAGGATTTATCCCGAGCCTGCTTGGAGACCTCCTAGAGGAGA |
| | GGCAGAAGATAAAGAAGAAGATGAAGGCCACGATTGACCTGATCGAGAGGAAGCTCCT |
| | CGATTACAGGCAGAGGCGTATCAAGATCCTGGCAAGCGGCTACTACGGTTACTACGGC |
| | TATGCAAGGGCGCGCTGGTACTGCAAGGAGTGTGCAGAGAGCGTAACGGCCTGGGGAA |
| | GGGAGTACATAACGATGACCATCAAGGAGATAGAGGAAAAGTACGGCTTTAAGGTAAT |
| | CTACAGCGACACCGACGGATTTTTTGCCCCAATACATGGAGCCGATGCTGAAACCGTC |
| | AAAAAGAAGGCTATGGAGTTCCTCAAGTATATCAACGCCAAACTTCCGGGCGCGCTTG |
| | AGCTCGAGTACGAGGGCTTCTACAAACGCGGCTTCTTCGTCACGAAGAAGAAGTATGC |
| | GGTGATAGACGAGGAAGGCAAGATAACAACGGGTGGACTTGAGATTGTGAGGCGTGGC |
| | TGGAGCGAGATAGCGAAAGAGACGCAGGCGAGGGTTCTTGAAGCTTTGCTAAAGGACG |
| | GTGACGTCGAGAAGGCCGTGAGGATAGTCAAAGAAGTTACCGAAAAGCTGAGCAAGTA |
| | CGAGGTTCCGCCGGAGAAGCTGGTGATCCACGAGCAGATAACGAGGGATTTAAAGGAC |
| | TACAAGGCAACCGGTCCCCACGTTGCCGTTGCCAAGAGGTTGGCCGCGAGAGGAGTCA |
| | AAATACGCCCTGGAACGGTGATAAGCTACATCGTGCTCAAGGGCTCTGGGAGGATAGG |
| | CGACAGGGCGATATCGTTCGACGAGTTCGACCCGGCGAAGCACAAGTACGACGCCGAG |
| | TACTACATTGAGAACCAGGTTCTCCCACCCGTTGAGAGAATTCTGAGAGCCEGCGGTT |
| | ACCGCAAGGAAGACCTGCGCTACCAGAAGACGAGACAGGTTGGTTTGAGTGCTTGGCT |
| | GACGCCGAAGGGAACTTGAGTCGACCTGCAGCCAAGCTTAATTAGCTGAGCTTGGACT |
| | CCT |

The vector backbone required for Gibson assembly was generated with 1× Q5 Buffer, 0.5 μM Vector-Fwd (Table 6), 0.5 μM Vector-Rvs, 5 ng of pGDR11 9°N-WT, 0.4 mM dNTPs, 0.5 μL Q5 DNA polymerase (0.02 U/μL). PCR was performed as followed: denature at 95° C. for 2.5 min followed by 30 cycles: denature at 95° C. for 30 sec, anneal at 68° C. for 45 sec, extended at 72° C. for 2.5 min and final step at 72° C. for 1 min. PCR reaction (2 μL) was visualized on a 1% ethidium-bromide agarose gel and run at 120 V for 45 min to confirm correct amplicon size. Zymo PCR cleanup was performed as followed: 100 μL of PCR reaction was mixed with 300 μL of DNA binding buffer and loaded onto Zymo IC column. The column was spun at 14,000 rcf for 30 sec and washed with 500 μL ×2 of Zymo wash buffer at 14,000 rcf for 30 sec. Column was further dried at 14,000 rcf for 3 min. DNA was eluted from column with 25 μL ×2 of nuclease free water and UV quantified.

TABLE 6
DNA oligonucleotides
Oligonucleotide sequences ordered from IDT written in the (5′→3′) direction.
Fluorescent dyes or modifications are noted on the specified termini. Highlighted in
bold are mismatched positions for sequences used for fidelity experiments.

| | | SEQ ID |
|---|---|---|
| | | NO: |
| Vector-Fwd | TGAGTCGACCTGCAGCCAAGCTTAATTAGCTGAGC | 9 |
| Vector-Rvs | GCTGCCGCGCGGCACCAG | 10 |
| 10-92-Fwd | ATCCATATGATCCTCGACACTGACTAC | 11 |
| 10-92-Rvs | ACGCATGCGGCCGCTCAAGTTCCTTTCGGCGTCAG | 12 |
| 10-92-Rvs_760 | ACGCATGCGGCCGCTCACTTCTGGTAGCGCAGGTC | 13 |
| 10-92-Rvs_750 | ACGCATGCGGCCGCTCAACCGCAGGCTCTCAG | 14 |
| Fragment1-Fwd | ATCACCATACGGATCCAAGCGGC | 15 |
| Fragment2-Fwd | TACGCCCTCCTGAAGGACGATTCT | 16 |
| Fragment3-Fwd | CAGGACGTCCCAGCGATAAGGGAC | 17 |
| Fragment4-Fwd | GCCTTCGCGATTGCGACTCTCTA | 18 |
| Fragment5-Fwd | AAGGGACGGATACACTTCGATCTCTAT | 19 |
| Fragment6-Fwd | GAGGCCCAGCTTTCTCGCTTA | 20 |
| Fragment7-Fwd | CCGGATACGCTCAACAGAGAAGGATGC | 21 |
| Fragment8-Fwd | GGCGTATCAAGATCCTGGCAAGCAGC | 22 |
| Fragment9-Fwd | CTTGAGCTCGAGTACGAGGGCTTCTAC | 23 |
| Fragment10-Fwd | GAGATAGCGAAAGAGACGCAGGCG | 24 |
| Fragment11-Fwd | CCGCCGGAGAAGCTGGTGATC | 25 |
| Fragment1-Rvs | AGAATCGTCCTTCAGGAGGGCGTA | 26 |
| Fragment2-Rvs | GTCCCTTATCGCTGGGACGTCCTG | 27 |
| Fragment3-Rvs | TAGAGAGTCGCAATCGCGAAGGC | 28 |
| Fragment4-Rvs | ATAGAGATCGAAGTGTATCCGTCCCTT | 29 |
| Fragment5-Rvs | TAAGCGAGAAAGCTGGGCCTC | 30 |
| Fragment6-Rvs | GCATCCTTCTCTGTTGAGCGTATCCGG | 31 |
| Fragment7-Rvs | GCTGCTTGCCAGGATCTTGATACGCC | 32 |
| Fragment8-Rvs | GTAGAAGCCCTCGTACTCGAGCTCAAG | 33 |
| Fragment9-Rvs | CGCCTGCGTCTCTTTCGCTATCTC | 34 |
| Fragment10-Rvs | GATCACCAGCTTCTCCGGCGG | 35 |
| Fragment11-Rvs | TAAGCTTGGCTGCAGGTCGACTCA | 36 |
| 30mer.V2HP.Cy3 | /5Cy3/ACAACCATTATCTAGAGCGATTCGTATAGGTGGTATCCGAAAG | 37 |
| | GATACCACC | |
| QP08.Iowa | ATGGTTGT/3IABkFQ/ | — |
| PBS8.20mer | /5IRD680/GTCCCCTTGGGGATACCACC | 38 |
| Template 4 | CATACGCGAGATACACTGCCAGAAAACCGCGTGTCTACGAGGTG | 39 |
| | GTATCCCCAAGGGGAC | |
| Random Library (30.5) | TCGTTCTCGGACGGAGAGATattgaa-N₄₀- | 40 |
| | GGTGGTATCCCCAAGGGGAC | |
| EM619 | CCCACACCCTCCTATCGCTAAACACACACTTAATAAAGTTGGTG | 41 |
| | GTATCC | |
| HIV-RT 2-1008 | AACAAAGTATCGAGAGACTGACGAAAAACATAAGAGTAGCGGTG | 42 |
| | GTATCC | |
| Bn 23 | GGCGAATTACTAACAGCGCGGGTGGTATCC | 43 |
| CMM595 | ACGCGCTTCTTGGAAACTCACGCACCCATAATAATACTAAGGTG | 44 |
| | GTATCC | |
| PBS37 | ATTGCTTGCCGGAGCTTATCGGAAGACTCACAGGCCTCCG | 45 |
| PBS33 | TCGTTCTCGGACGGAGAGAT | 46 |
| PBS38 | ATTGCTTGCCGGAGCTTATC | 47 |
| CMM595.Fidelity | TCGTTCTCGGACGGAGAGATACGCGCTTCTTGGAAACTCACGCA | 48 |
| | CCCATAATAATACTAACGGAGGCCTGTGAGTCAACC/3InvdT/ | |
| VectorFwd_KodRSGA* | ATTAGCTGAGCTTGGACTCCTGTTGATAGATCCAGT | 49 |
| VectorRvs_KodRSGA* | GGCCTCCATCGGAAGGAACTCCTTCCC | 50 |

gBlocks were reconstituted at the recommended volume with nuclease free water, mixed with 100 ng (1 μL) of the vector backbone and assembled through NEB Gibson assembly at 55° C. for 1 h. Transformation of Gibson assembly was performed as followed: 1 μL of Gibson reaction was transformed into 50 μL NEB 5-alpha competent E. coli and incubated on ice for 30 min. Prior to heat shock, 28 μM of BME was added and mixed with cells. Immediately, cells were heat shocked at 42° C. for 30 sec and immediately placed back on ice for 2 min. Cells were recovered with 1 mL of Super Optimal broth (SOC) and placed into a shaking incubator at 225 rpm for 1 h at 37° C. Recovered cells (250 μL) were plated onto 50 ng/ml carbenicillin agar plates. Plates were incubated overnight at 37° C. Individual colonies were picked and colony PCR was performed as followed: denature at 95° C. for 2 min followed by 30 cycles: denature at 95° C. for 15 sec, anneal at 58° C. for 15 sec, extend at 72° C. for 2.5 min final step at 72° C. for 1 min with Fragment1-Fwd (Table 6) and Fragment11-Rvs. PCR reactions were analyzed by 1% ethidium bromide agarose gels to verify the correct amplicon size. Sanger sequencing was performed to verify the sequence composition of each gene. Site directed mutagenesis was performed with Q5 to repair improper nucleotide mutations. Post-repair PCR reactions were analyzed by 1% ethidium bromide agarose gel to verify full vector amplicon. PCR reaction (1 μL) was treated with NEB KLD, transformed, and plated on 50 ng/ml carbenicillin agar plates. Colony PCR was performed, and gene-containing colonies were sequenced by Sanger sequencing.

A total of 44 fragment cassettes were individually generated by PCR using primers (Table 6) that were specific to internal regions of each gene. PCR reactions contained a final concentration of 1× Q5 buffer, 0.5 μM Fwd primer, 0.5 μM Rvs primer, 0.4 mM dNTPs, and 0.5 μL Q5 DNA polymerase (0.02 U/μL) in a final volume of 50 μL. PCR cycles were performed as followed: denature at 95° C. for 2 min followed by 30 cycles: denature at 95° C. for 30 sec, anneal at 68.6° C. for 45 sec, extend at 72° C. for 2.5 min and a final step at 72° C. for 1 min. Each fragment was mixed with Zymo DNA binding buffer and loaded onto Zymo IC column for purification. DNA was washed 4× 500 μL DNA wash buffer (10 mM Tris-HCl, pH 7.4, 80% ethanol). DNA was eluted from the column with 2× 25 μL of nuclease free water and UV quantified.

DNA shuffling of the 44 gene fragments proceeded by PCR. First, each version of fragment 1 (Kod, Tgo, 9N, DV) were mixed (25 ng each) to produce 100 ng of DNA in a 10 μL volume, generating a 10 ng/μL working stock for assembly. This process was repeated for each gene fragment region. Mass ratios between the fragment sizes and the entire gene were used to calculate the amount required for the overlap PCR. For a gene size of 2,631 base pairs, we used a set concentration of 263.1 ng for a 50 μL overlap PCR assembly. For example, fragment 1 cassette is 179 base pairs out of 2,631 base pairs, which is 6.8% of the entire assembled gene. Therefore, 1.79 μL of DNA was added from the 10 ng/μL working stock for fragment 1. The process was repeated for the remaining fragments. The remaining components added for the overlap PCR were 1× Q5 buffer, 0.4 mM dNTPs, 0.5 μL of Q5 DNA polymerase (0.02 U/μL) in the absence of primers. The overlap PCR conditions were performed as followed: denature at 94° C. for 30 sec, followed by 9 cycles of denature at 94° C. for 15 sec, extend at 72° C. for 1.5 min with a decrease of 0.5° C. per cycle. Then 5 cycles of denature at 94° C. for 15 sec, anneal for 67.5° C. for 15 sec with a decrease of 0.5° C. per cycle, extend at 72° C. for 1.5 min, with a final polishing step of 72° C. for 2 min.

To amplify full length genes, 4 μL of the overlap PCR assembly mixture was combined with 1× Q5 buffer, 0.4 mM dNTPs, 0.5 μL of Q5 DNA polymerase and the presence of 0.5 μM Fragment1-Fwd and 0.5 μM Fragment11-Rvs primers. The PCR conditions for 40 cycles were as followed: denature at 94° C. for 30 sec, followed by 17 cycles of denature at 94° C. for 15 sec, anneal at 67° C. for 15 sec with a decrease of 0.5° C. per cycle, extend at 72° C. for 1.5 min. Then 23 cycles of denature at 94° C. for 15 sec, anneal at 59° C. for 15 sec, extend at 72° C. for 1.5 min, with a final polishing step of 72° C. for 2 min. After PCR, samples (2 μL) were analyzed on a 1% ethidium-bromide agarose gel at 120 V for 45 min.

Once full-length amplicons were generated, the PCR product was purified by 1% ethidium-bromide agarose gel at 70 V for 120 min. The corresponding band was excised and dissolved with Zymo agarose dissolving buffer (100 mg of agarose to 300 μL Zymo agarose dissolving buffer) by incubating at 37° C. for 1 h. Dissolved solution was loaded into a Zymo IC column and spun at 14,000 rcf for 30 sec. DNA wash buffer (500 μL) was added to the column, mixed by inversion, and removed. The interior wash was repeated for a total of 2×, followed by exterior washes to the outside of the column by rinsing with 1 mL×4 of DNA wash buffer. Following exterior column washes, the column was placed into a fresh collection tube, and 500 μL×4 of DNA wash buffer was spun through the column at 14,000 rcf for 30 sec. The column was further dried at 14,000 rcf for 3 min. DNA was eluted with 10 μL×2 of nuclease free water and UV quantified.

Gibson assembly was utilized (see above) to construct the expression vector using the newly generated homologous recombination library (100 ng) and the previously generated vector backbone (100 ng). Transformation was subsequently performed as described above. Colony PCR and Sanger sequencing was performed to validate the generation of the recombined library and to monitor parental plasmid contamination. Upon validation, the Gibson assembled material was scaled for a total of 5 transformations into NEB 5-alpha competent E. coli. Colonies were scrapped from agar plates and purified using the Plasmid Midiprep II kit following the manufacturer's recommended instructions. Plasmid DNA was eluted in nuclease free water, UV quantified and stored for future transformation into XL1-blue E. coli for DrOPS sorting and polymerase expression.

Preparing E. coli for Encapsulation in Droplets

A detailed description of photolithography and microfluidic device fabrication for DrOPS sorting has been previously described 3,4. Cells expressing the recombinant polymerase library were prepared by transforming 300 ng of the plasmid library into 20 μL of XL1-blue E. coli. DNA was incubated with cells for 30 min on ice, heat shocked at 42° C. for 30 sec and immediately placed on ice for 2 min. SOC (1 mL) was added to the cells and placed into a shaker at 37° C. with shaking at 225 rpm for 1 h. Recovered cells were then used to inoculate 50 mL of LB-carbenicillin (50 μg/mL) liquid medium in a 250 mL baffled flask. The liquid culture was grown to confluency overnight at 37° C. with shaking at 225 rpm. Overnight (500 μL, 1:100 v/v) was used to inoculate 50 mL of LB-carbenicillin (50 μg/mL) liquid medium in a 250 ml baffled flask and grown at 37° C. with shaking at 225 rpm. At an OD600 of ~0.6 au, the culture was cooled to 25° C., induced with 1 mM IPTG, and incubated overnight at 25° C. with shaking at 225 rpm. After expression, 1 mL of cell culture was collected and centrifuged for 5 min at 3,220 rcf, and the supernatant was discarded. The cells were washed with 1 mL of commercial 1× ThermoPol buffer (20 mM Tris-HCl, 10 mM (NH₄)₂SO₄, 10 mM KCl, 2 mM MgSO₄, 0.1% TritonX-100, pH 8.8) for a total of four washes (4 mL). The bacterial pellet was suspended in 2 mL of 1× ThermoPol buffer and the absorbance was measured at 600 nm. Cells were diluted to an OD of 0.05 to enable encapsulation at occupancies of 0.1 cells per droplet according to a Poisson distribution. Just prior to emulsification, the cells were mixed with the reagents for the polymerase activity assay (PAA). The polymerase activity assay consists of 1 μM of a self-priming hairpin template labeled with Cy3 at the 5′ end (30merHP.V2, Table 6), 2 μM of a 3′ end labeled Iowa Black quencher sequence (QP08.Iowa, Table 6), and 100 μM of TNA triphosphates (INTPs) in 1× ThermoPol buffer.

All aqueous and oil solutions were sealed as individual 1.5 mL plastic micro-centrifuge tubes and controlled via pressure driven flow with custom LabVIEW software (National Instruments). Two lengths of Tygon tubing were inserted through holes drilled into the caps of the micro-centrifuge tubes and glued into place to create an airtight seal. One length of tubing remained in the pressure headspace above the reagent and was connected at the other end to a SMC ITV0011-2UMS digital pressure regulator). Another length of tubing was submerged in the reagent solution with the other end connected to the appropriate inlet of the microfluidic device. By manually applying a positive pressure head to the reagent vial via the SMC digital regulator, fluid was driven through the channels of the microfluidic device. A length of Tygon tubing was also inserted in the outlet and placed in a micro-centrifuge tube for droplet collection.

All emulsions were produced using custom PDMS chips^{3,4}. Single emulsions were formed utilizing a flow focusing geometry. The aqueous phase containing the polymerase activity assay and E. coli cells was sheared by a continuous phase consisting of a low-viscosity fluorinated oil (HFE-7500) containing 1% (w/w) Pico-Surf surfactant. Pressures were maintained to achieve droplet diameters of ~20-22 μm and production rates of 30-35 kHz, allowing 110-125×10⁶ droplets to be produced every hour. Single emulsions were collected under a layer of mineral oil in 1.5 mL plastic micro-centrifuge tubes and incubated for 5 mins at 95° C. to lyse the cells, followed by incubation at 55° C. for various times.

### FADS Sorting of Single Emulsion Droplets

A detailed schematic and operation of Fluorescence Activated Droplet Sorter (FADS) instrumentation was previously described^{3,4}. In brief, following incubation, droplets were injected into a FADS device capable of droplet sorting. Incident light from a 552 nm laser was focused through a 20× plan apochromatic objective (Motic, Hong Kong) where droplets pass in single file. Emitted light was led through a 562 nm Quad Band Dichroic into an optical train through a series of long-pass dichroics to a photomultiplier tube (PMT). The sample was illuminated with blue light to not overlap with the spectral properties of Cy3 and was imaged with a high-speed camera at 35,000 frames per second (fps). The digital signals generated by the PMT were analyzed by a field-gated programmable array (FPGA) controlled with custom LabView software. Droplets falling within a user-defined threshold triggered the FPGA to send a square-wave pulse (50 kHz, 50% duty cycle, 60 μs), amplified to 600 V by a high-voltage amplifier, to the salt-water electrode (4 M NaCl) of the sorting chip. The resulting nonuniform electric field generated a dielectrophoretic (DEP) force that polarized and deflected the droplet into a collection channel.

### Recovery of Sorted DNA and Library Regeneration

Plasmid DNA was recovered from the population of positively sorted droplets present in the collection tube by extraction with Pico-Break following the manufacturers recommended protocol. DNA samples were recovered from sorted emulsions by extraction with 2 volumes of Pico-Break, which contains 1H,1H,2H,2H-perfluorooctanol (PFO). After addition of Pico-Break 1, the samples were vortexed and centrifuged (60 sec, 2,000×g) to attain phase separation. The aqueous layer (top) containing the plasmid DNA was recovered. The bottom layer was extracted a second time with 1 volume of nuclease free water to improve recovery yields. The combined aqueous layers containing the plasmid DNA were concentrated using a Zymo IC column, washed with 500 μL×2 DNA wash buffer and eluted with nuclease free water (20 μl). The gene was amplified in 1× Q5 buffer, 0.5 μM fragment1-Fwd (Table 6), 0.5 μM fragment11-Rvs 0.4 mM dNTPs, 4 μL of recovered DNA, and 0.5 μL Q5 DNA polymerase (0.02 U/μL) in a total volume of 50 μL. PCR amplification was performed as followed: denature at 95° C. for 2.5 min followed by 30 cycles: denature at 95° C. for 30 sec, anneal at 70° C. for 45 sec, extend at 72° C. for 2.5 min followed by a polishing step of 72° C. for 1 min. The PCR reaction (2 μL) was run on a 1% ethidium-bromide agarose gel for amplicon validation.

Upon validation of amplification, samples were pulled and mixed with 3× Zymo DNA binding buffer (i.e., 100 μL PCR reaction to 300 μL Zymo DNA binding buffer) and added to a Zymo IC column. DNA was cleaned with 500 μL×4 DNA wash buffer at 14,000 rcf for 30 sec. The Zymo IC column was further dried at 14,000 rcf for 3 min prior to elution. DNA was eluted from column with 20 μL×2 of nuclease free water at 14,000 rcf for 1 min. The addition of 5 μL NEB rCutSmart buffer and 5 μL of NEB DpnI was mixed with DNA to digest methylated and hemi-methylated parent plasmid for 4 h at 37° C. followed by heat inactivation of the enzyme at 80° C. for 20 min. Post DpnI treatment, 15 μL of NEB 6× Blue Gel loading dye was mixed with the DpnI treated sample and loaded into a 1% ethdium-bromide agarose gel run at 70 volts for 120 min to separate gene amplicon and digested parental plasmid.

The gene amplicon band was excised and dissolved in Zymo agarose dissolve buffer at 37° C. for a minimum of 1 h. The dissolved gel solution was added to a Zymo IC column and spun at 14,000 rcf for 30 sec. The columns interior and exterior were rinsed with DNA wash buffer as previously mentioned. Columns were washed with addition 4×300 μL of DNA wash buffer. Samples were dried at 14,000 rcf for 3 min prior to the addition of 20 μL of nuclease free water and spun at 14,000 rcf for 1 min. DNA was UV quantified on a NanoDrop instrument.

For the Gibson assembly, equimass quantities of each (100 ng) vector and insert DNA are used following the manufacturer's recommended protocol with the exception of an extended incubation time (1 h). Post assembly, reaction mixture was mixed with 3× Zymo DNA binding buffer, loaded into Zymo IC column and spun at 14,000 rcf for 30 sec. The Zymo IC column wash with 4×500 μL DNA washed buffer at 14,000 rcf for 30 sec. Prior to elution the Zymo IC column was further dried at 14,000 rcf for 3 min. DNA was eluted with 20 μL of nuclease free water at 14,000 rcf for 1 min. A transformation was performed as previously mentioned using 1 μL of Gibson assembled material.

### Construction of Mutagenic Library

Mutagenic PCR was performed using Kod-WT (exo-) DNA polymerase, which harbors two exonuclease silencing mutations (D141A, E143A) and is preferred for blunt ended amplification. The polymerase was expressed and purified from E. coli as described previously⁵. The activity of Kod-WT (exo-) polymerase was empirically determined by serial diluting the enzyme in PCR reactions, from which a resulting 0.013 μM concentration of polymerase was determined suitable for PCR. The mutagenic PCR was performed with biased dNTP ratios, and supplemented MnCl₂ and MgCl₂ as described previously^{6,7}. The PCR reaction was performed with the following final concentration: 1× ThermoPol, 1 μM forward primer, 1 μM reverse primer, 1 mM TTP, 1 mM dCTP, 0.2 mM dGTP, 0.2 mM dATP, 5.5 mM MgCl₂, 0.013 μM Kod-WT (exo-) polymerase, 100 ng of 5-270 plasmid and 2 fold serial dilution of MnCl₂ from 500-31.25 μM. PCR cycles were performed as followed: denature at 95° C. for 2 min followed by 30 cycles of denature at 95° C. for 1 min, anneal at 60° C. for 1 min, extend at 72° C. for 2.5 min, and followed by agarose gel analysis. Successful PCR reactions are consolidated for a PCR cleanup, DpnI treatment, and agarose gel purification. The purified DNA and the corresponding vector backbone were combined in a Gibson assembly reaction, column purified, and transformed as described above. Single colonies were picked, grown, miniprepped and sequenced by Sanger sequencing. Mutagenic PCR can be performed with any combination of forward and reverse primer set as examples: Fragment9-Fwd and Fragment11-Rvs for the thumb subdomain and Fragment6-Fwd and Fragment8-Rvs for the palm and finger subdomains.

### Colony Picking and Polymerase Activity Screen

Protocol for lysate activity screen was performed as described 8. In brief, Gibson assembly material from post regeneration were transformed into XL1 Blue E. coli, recovered and plated onto agar plates containing 50 ng/μL carbenicillin. Single colonies were picked and placed into 4 mL of LB media containing 50 ng/μL carbenicillin and grown in a shaking incubator at 225 rpm, overnight at 37° C. Overnight culture (40 μL) was inoculated into fresh 4 mL LB media containing 50 ng/μL carbenicillin and grown to an OD₆₀₀~0.6. Cells were induced with 1 mM IPTG and expressed at 25° C., 225 rpm for 18-20 h. Cells were harvested, centrifuged (4000 rpm, 25° C., 10 min) and suspended in 10 mM Tris-HCl, pH 8.0, 500 mM NaCl and 10% glycerol and transferred to 1.5 mL centrifuge tubes. Tubes were placed into 70° C. Eppendorff ThermoMixer with heated lid for 1 h, cooled for 1 h on ice, and centrifuged (13200 rpm, 4° C., 1 h). Clarified supernatant was assessed with Bradford dye against 10, 5, and 0 μM of purified parent polymerase.

Polymerase activity assay was performed with the following final concentrations: 1× ThermoPol, 0.5 μM of template 4, 0.5 μM of 5′-IR800-primer PBS8.20mer, 100 μM tNTPs, and 1 or 2 μL of lysate in a final volume of 20 μL. Primer-template were annealed in 1× ThermoPol at 90° C. for 5 min and immediately placed on ice for 5 min, followed by the addition of tNTPs and lysate on ice. Reactions were placed into thermal cycler at 55° C. At designated time points 1 μL of reaction was removed, mixed with 39 μL of quenching buffer (95% formamide, 25 mM EDTA) and denatured at 95° C. for 10 min. Denatured samples were loaded (10 μL) into a 15% urea denaturing PAGE and run at 10 W for 1.5 h and visualized on Li-Cor Odyssey CLx Imager.

### Recombinant Polymerase Expression and Purification of Novel TNA Polymerases

Protein expression and purification was performed as previously described 5. In brief, pGDR11 vector containing the variant of interest was transformed into XL1 Blue E. coli, grown, induced for expression, and harvested. Cells were suspended in buffer (10 mM Tris-HCl, pH 8.0, 500 mM NaCl, 10% glycerol), sonicated, heat treated at 70° C. for 1 h, and cooled on ice for 1 h. The solution was centrifuged (20000 rpm, 4° C., 20 min) and clarified supernatant was treated with a final concentration of 0.5% (v/v) PEI for 15 min. Treated supernatant was centrifuged (20000 rpm, 4° C., 20 min) to precipitate nucleic acids. Ammonium sulfate precipitation was performed (final concentration: 60% (w/v)), centrifuged (20000 rpm, 4° C., 20 min), and the protein pellet was resuspended in equilibration buffer (10 mM Tris-HCl, pH 8.0 50 mM NaCl, 10% glycerol). Polymerase was purified by hand on a 5 mL heparin affinity column and eluted by stepwise addition of buffers containing: 10 mM Tris-HCl, pH 8.0, 10% glycerol and increasing concentration of NaCl (50, 100, 250, 500, 750 mM).

### Thermostability Challenge

Purified polymerase was quantified to 10 μM and aliquoted (100 μL) into 6, 1.5 mL Eppendorf tubes and placed into an Eppendorff ThermoMixer set 90° C. with a thermal lid to prevent evaporation. At the designated time (1-6 h) a tube was removed and placed on ice until activity evaluation. The collected tubes were spun at 12,000 rpm for 5 min at 4° C. and UV quantified using a NanoDrop instrument. The polymerase was assayed for activity of TNA synthesis across a DNA template. Polymerase activity screen was performed with the following final concentrations: 1 μM primer-template duplex, 100 μM tNTPs, 1 μM heat-treated Polymerase in a 20 μL reaction. After 30 minutes of incubation, a 1 μL aliquot of the reaction was quenched by the addition of 39 μL of quenching buffer and visualized by denaturing polyacrylamide gel electrophoresis with fluorescent imaging on a Li-Cor Odyssey CLx Imager.

### Substrate Recognition

TNA polymerases were evaluated for the ability to recognize and incorporated C5′-modified uracil TNA triphosphates (Trp and Phe) previously generated by chemical synthesis⁹. Polymerase activity screen was performed as described above with the exception of 2 equivalents of polymerase and combination of the C-5 modified tUTP substrate with standard tCTP, tGTP, and tATP substrates for a final concentration of 100 μM.

### Synthesis and Preparation of TNA Aptamers for BLI Analysis

Aptamer generation for BLI analysis was performed as described 10. In brief, the DNA templates encoding the TNA aptamers were ordered with the addition of the PBS8 primer binding site on the 3′ termini. The DNA template (1.2 μM) was annealed to a biotinylated-PBS8 primer (1 μM) in 1× ThermoPol at 90° C. for 5 min and immediately snap cooled, followed by the addition of 100 μM of respective TNA base mixes and 2 μM of 10-92 for a total volume of 750 μL. Primer extension was performed at 55° C. for 2 h. The reaction was quenched by freezing the sample, vacuum concentrating to a volume of <100 μL and adding 250 μL of quenching buffer. The quenched sample was heated to 90° C. for 5 min and loaded onto a 15% urea-denaturing polyacrylamide gel and ran at 15W for 2.5 hrs. The product band was excised, electroeluted, buffer exchanged into water and UV quantified. For BLI analysis, the aptamer was diluted to 100 nM final concentration (1 mL) with BLI binding buffer (10 mM HEPES pH 7.0, 150 mM NaCl, 3 mM EDTA, 0.05% Tween-20) heated to 90° C. for 5 minutes and snap cooled. The protein target was prepared by performing 2-fold serial dilutions starting from 100 nM to obtain the following concentrations: 25, 12.5, 6.25, and 3.125 nM.

### BLI Analysis of Selected TNA Aptamers

Aptamer sequences of interest were characterized with four different concentrations of target and one buffer only sensor to determine the background. Prior to testing, all sensors were equilibrated in BLI binding buffer for at least 30 min. After aptamer folding, the BLI run was performed with the following steps: a buffer only baseline 60 s to equilibrate sensors, loading the aptamer for 200 s, a second buffer only baseline for 200 s, an association phase with the target protein for 600 s, and a dissociation phase for 600 s. The plate and reagents were incubated at 30° C. for the duration of the experiment and for 10 min prior to each run. Data was analyzed using the Octet Data Analysis HT software. For full kinetic measurements, the buffer only baseline sample was used to subtract the background from all samples before applying Savitzky-Golay filtering and fitting both the association and dissociation curves together and applying a global fit to determine K_{D} and other metrics.

### Fidelity Assay of Novel TNA Polymerase

Fidelity analysis of the novel 10-92 TNA polymerase was performed as previously described¹¹. In brief, a primer extension reaction was performed using a DNA primer (1 μM Extra_PBS37) containing a two-nucleotide mismatch (AA-AA) annealed to a DNA template (1 μM CMM595). The primer extension reaction was performed for 1 h at 55° C. in 1× Thermopol buffer, 100 μM tNTPs, and 2 μM 10-92 TNA polymerase in a 25 μL reaction. The extension product was purified by denaturing PAGE and reverse transcribed into cDNA. The reverse transcription reaction was performed in 1× ThermoPol with 2 μM Bst-LF DNA polymerase, 2 μM PBS33, 0.4 mM dNTPs in a final volume of 20 μL and incubated for 4 h at 50° C. The cDNA product was PCR amplified with Taq DNA polymerase (PBS33 and PBS38) and agarose gel purified. The purified DNA was ligated into a TOPO vector and transformed into E. coli DH5-α competent cells. Colony PCR was performed on individual colonies to verify inserted amplicon, grown in liquid media, miniprepped and sequenced by Sanger sequencing with the M13F universal primer. DNA sequences were aligned to the template and analyzed using MEGA11 software.

### Cloning of Kod-RSGA+DE Mutations (R6-10) Construct for Neutral Drift Validation

The 10 mutations acquired by directed evolution were cloned into the starting backbone of Kod-RSGA, creating the construct Kod-RSGA*. A gBlock was ordered to contain the linear dsDNA sequence encompassing the 10 mutations (Table 5, Kod-RSGA* partial construct). Corresponding primers (Table 6, Vector Fwd KodRSGA* and Vector Rvs KodRSGA*) were ordered to facilitate the amplification of the remaining gene and expression vector from the original pGDR11-kod-RSGA construct. The resulting vector portion was used after a DNA clean up, DpnI treatment, and agarose gel purification. The insert and vector were ligated through Gibson assembly as described above. The resulting construct was validated by Sanger sequencing, expressed in E. coli, and purified as described above, and challenged in a primer extension.

### Polymerase Kinetics

TNA polymerase kinetic measurements were performed by monitoring the reaction progress over time as the DNA primer-template (P-T) duplex (PBS8 & EM619, Table 6) was extended with tNTPs. For each time point, 3 individual reaction replicates (15 μL) from a single master mix poised under single-turnover conditions with equimolar concentrations of polymerase (0.2 μM) and pre-annealed P-T duplex (0.2 μM each, heated at 90° C. for 5 min, cooled on ice) in ThermoPol buffer (NEB, 2×) were pre-equilibrated for 5 minutes at 55° C. The reactions were then initiated by the addition of a preheated tNTPs mixture (200 μM of each tNTP). The reactions were stopped at designated time points by plunging the reaction vessel into powdered dry ice. Once all the time points were collected, the frozen reactions were thawed on ice by adding 15 μL (6 μM) EvaGreen® dye, previously identified as the optimal intercalating dye to monitor synthesis of TNA on a DNA template¹². Each reaction (25 μL) was transferred to a clear V-bottom 96-well plate and fluorescence intensity was measured (ex: 490 nm, em: 530 nm) using CLARIOstar Plus instrument.

Baseline fluorescence was measured using Kod-WT (exo-), the parent polymerase with limited TNA synthesis ability 13, which was used to collect a 0 second time point by freezing the reaction immediately after the addition of the tNTP solution. Additionally, the maximum fluoresce value was obtained from a 2 hour reaction performed with the 10-92 TNA polymerase. The raw fluorescence data from the plate reader was normalized by subtracting the baseline and then dividing by the difference of the maximum (2 hour reaction with 10-92) and minimum (baseline) fluorescence values. The fluorescence data was then converted to nucleotides per polymerase as previously described 12 by multiplying data with the conversion factor F=[Template]*L/[Polymerase], where L is the length of extension in bases, then plotted over time. Rates in nucleotides per minute are extracted as the slope of the linear range of each curve.

The fluorescence-based kinetic measures were validated by denaturing PAGE using an IR-680 labelled DNA primer. Frozen reactions were thawed with 105 μL of 95% formamide and 25 mM EDTA, heated for 10 minutes at 90° C., analyzed by denaturing 15% Urea-PAGE, and imaged on Odyssey CLx LI-COR imager.

### Cloning of Truncated Polymerases for Crystallography

The 10-92 gene was PCR amplified from pGDR11-10-92 using the 10-92-Fwd and 10-92-Rvs primers (Table 6) containing NdeI and NotI restriction enzyme sites, respectively. Two 10-92 C-terminal truncations, 10-92_760 and 10-92_750, were PCR amplified by substituting the 10-92-Rvs primer with either 10-92-Rvs_760 or 10-92-Rvs_750 primer (Table 6), both containing the NotI restriction enzyme site. All three gene constructs additionally consist of two mutations (D141A and E143A) to inactivate exonuclease activity. Purified PCR product of each gene construct and pET21 were digested with NdeI and NotI restriction enzymes and ligated, resulting in pET21-10-92, pET21-10-92_760, and pET21-10-92_750. The three constructs were verified by Sanger sequencing.

### Sample Preparation and Crystallization

The DNA template was purchased from IDT as an HPLC purified sample. The DNA primer was also ordered from IDT with standard desalting. The primer-template (P/T) duplex was prepared by combining equal parts of the primer and template strands in Kod buffer (50 mM Tris-HCl pH 8.5, 200 mM NaCl, 0.1 mM EDTA, 1 mM DTT) and supplemented with 20 mM MgCl₂, heating at 95° C. for 5 min and slow cooling to 10° C. over 10 min. An initial binary complex was prepared by incubating 10-92_760 (6 mg mL⁻¹) with 1.2 M equivalents of the annealed P/T duplex at 37° C. for 30 min. 3 M excess of dtTTP was added and incubated at 37° C. for 30 min. The resulting n+1 binary complex was further incubated with 10 M excess tATP at 37° C. for another 30 min to yield the final ternary complex.

Next, the 10-92 ternary complex was screened against ~900 conditions in a hanging-drop format using a Mosquito crystallization robot (SPT LabTech). A positive crystal hit was initially identified in condition C9 in a NeXtal PACT Screen and was further optimized in 24-well hanging drop trays over a range of pH and PEG concentrations, with each drop consisting of 1 μL of sample mixed with 1 μL of mother liquor over 500 μL mother liquor in every well. Trays were stored in the dark at room temperature and crystals typically grew between 3-4 days. The 10-92 ternary complex crystal used for structure determination was grown in 0.2 M lithium chloride, 0.1 M HEPES pH 6.5, and 35% polyethylene glycol 6000.

### Structure Determination

A 2.73 Å dataset was collected at the Advanced Light Source (Lawrence Berkeley National Laboratory, Berkeley, CA) from a single crystal. Images were indexed, integrated, and merged using XDS^{14,15}. The initial model was determined by molecular replacement using Phaser¹⁶ with PDB structure 5VU8 as the search model, and the final model was determined using iterative rounds of manual building through Coot¹⁷ and refinement with phenix¹⁸. The stereochemistry and geometry of all structures were validated with Molprobity¹⁹ with the final refinement parameters summarized in Table 3. Final coordinates and structure factors have been deposited in the Protein Data Bank. All molecular graphics were prepared with PyMOL²⁰.

### REFERENCES

1 Sau, S. P., et al. J. Org. Chem. 81, 2302-2307, (2016).
2 Liao, J.-Y., et al. J. Am. Chem. Soc. 141, 13286-13289, (2019).
3 Vallejo, D., et al. ACS Synth. Biol. 8, 1430-1440, (2019).
4 Vallejo, D., et al. Methods Enzymol. 644, 227-253, (2020).
5 Nikoomanzar, A., et al. Curr. Protoc. Nucleic Acid Chem. 69, 4.75, (2017).
6 Cadwell, R. C. & Joyce, G. F. PCR Methods Appl. 2, 28-33, (1992).
7 Chaput, J. C. & Szostak, J. W. Chem. Biol. 11, 865-874, (2004).
8 Nikoomanzar, A., et al. ACS Synthic Biology 9, 1873-1881, (2020).
9 Li, Q. et al. J. Am. Chem. Soc. 143, 17761-17768, (2021).
10 McCloskey, C. M. et al. ACS Synth. Biol. 10, 3190-3199, (2021).
11 Nikoomanzar, A., et al. ACS Synth. Biol. 8, 1421-1429, (2019).
12 Nikoomanzar, A., et al. Anal. Chem. 89, 12622-12625, (2017).
13 Medina, E., et al. ACS Synth. Biol. 10, 1429-1437, (2021).
14 Kabsch, W. Xds. Acta Crystallogr. D Biol. Crystallogr. 66, 125-132, (2010).
15 Kabsch, W. Acta Crystallogr. D Biol. Crystallogr. 66, 133-144, (2010).
16 McCoy, A. J. et al. Phaser crystallographic software. J. Appl. Crystallogr. 40, 658-674, (2007).
17 Emsley, P., et al. Acta Crystallogr. D Biol. Crystallogr. 66, 486-501, (2010).
18 Afonine, P. V. et al. Acta Crystallogr. D Biol. Crystallogr. 68, 352-367, (2012).
19 Chen, V. B. et al. Acta Crystallogr. D Biol. Crystallogr. 66, 12-21, (2010).
20 The PYMOL molecular graphics program (DeLano Scientific, San Carlos, CA, USA, 2002).
21 Badaczewska-Dawid, A. E., et al. Nucleic Acids Res. 50, W474-W482, (2022).
22 Sagendorf, J. M., et al. Nucleic Acids Res. 48, D277-D287, (2020).
23 Zheng, G., et al. Nucleic Acids Res. 37, W240-246, (2009).

### Representative Embodiments

The following embodiments are representative of compositions and methods provided by the present disclosure. One exemplary embodiment is a purified threose nucleic acid (TNA) polymerase comprising an amino acid sequence at least 95% identical to SEQ ID NO: 2, wherein the amino acid sequence comprises each of the residues indicated in FIG. 2A; and wherein the encoded DNA polymerase synthesizes a TNA in the presence of a DNA template and threose nucleotides. In one exemplary embodiment, the amino acid sequence is at least 98% identical to SEQ ID NO: 2. In one exemplary embodiment, the amino acid sequence is SEQ ID NO: 2.

Another exemplary embodiment is a nucleic acid encoding the TNA polymerase comprising an amino acid sequence at least 95% identical to SEQ ID NO: 2, wherein the amino acid sequence comprises each of the residues indicated in FIG. 2A; and wherein the encoded DNA polymerase synthesizes a TNA in the presence of a DNA template and threose nucleotides. In one exemplary embodiment, the nucleic acid is an expression vector. Another exemplary embodiment is a recombinant cell comprising the nucleic acid expression vector. In one exemplary embodiment, the recombinant cell is a prokaryotic cell.

Another exemplary embodiment is a kit comprising the purified TNA polymerase comprising an amino acid sequence at least 95% identical to SEQ ID NO: 2, wherein the amino acid sequence comprises each of the residues indicated in FIG. 2A; and wherein the encoded DNA polymerase synthesizes a TNA in the presence of a DNA template and threose nucleotides. The kit further comprises at least one threose nucleotide. In one exemplary embodiment, the at least one threose nucleotide comprises tA, tT, tG, and tC.

Another exemplary embodiment is a method for synthesizing a TNA, the method comprising contacting a DNA template with: (i) a purified TNA polymerase comprising an amino acid sequence at least 95% identical to SEQ ID NO: 2, wherein the amino acid sequence comprises each of the residues indicated in FIG. 2A; and wherein the encoded DNA polymerase synthesizes a TNA in the presence of a DNA template and threose nucleotides; and (ii) a plurality of threose nucleotide triphosphates. The contacting occurs under conditions that permit TNA polymerization, whereby a TNA is synthesized by the TNA polymerase. In one exemplary embodiment, the amino acid sequence is at least 98% identical to SEQ ID NO: 2.

Another exemplary embodiment is a method for synthesizing TNA oligonucleotides, the method comprising contacting a DNA template with: (i) a purified TNA polymerase comprising an amino acid sequence at least 95% identical to SEQ ID NO: 2, wherein the amino acid sequence comprises each of the residues indicated in FIG. 2A; and wherein the encoded DNA polymerase synthesizes a TNA in the presence of a DNA template and threose nucleotides; and (ii) a plurality of threose nucleotide triphosphates, wherein at least one threose nucleotide triphosphate comprises a modified base. The contacting occurs under conditions that permit TNA polymerization, whereby a TNA comprising a modified base is synthesized by the TNA polymerase. In one exemplary embodiment, the amino acid sequence is at least 98% identical to SEQ ID NO: 2. In one exemplary embodiment, the modified base comprises a C5-modified tUTP or tCTP monomer or C7-modified 7-deaza-tGTP or 7-deaza-tATP monomer bearing side chains. In one exemplary embodiment, the side chains are selected from phenylalanine (Phe), tryptophan (Trp), tyrosine, cyclopropyl, naphthalene, and isoleucine.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A threose nucleic acid (TNA) polymerase comprising an amino acid sequence at least 80% identical to SEQ ID NO: 2, wherein the amino acid sequence comprises each of the residues R99, A102, V107, I127, T136, A141, A143, K285, A296, Q297, G304, V337, H339, P340, Y356, R375, Y377, E378, L381, E383, A386, K395, R466, V472, L474, L475, K477, R486, S492, G493, Q520, E523, T524, R527, F533, L538, A540, P548, H550, K562, D566, L575, D602, G607, G615, R672, S717, A724, P741, C749, and T771 of SEQ ID NO: 2; and wherein the encoded DNA polymerase synthesizes a TNA in the presence of a DNA template and threose nucleotides.

2. The TNA polymerase of claim 1, wherein the amino acid sequence is at least 85% identical to SEQ ID NO: 2.

3. The TNA polymerase of claim 1, wherein the amino acid sequence is at least 90% identical to SEQ ID NO: 2.

4. The TNA polymerase of claim 1, wherein the amino acid sequence is at least 95% identical to SEQ ID NO: 2.

5. The TNA polymerase of claim 1, wherein the amino acid sequence is SEQ ID NO: 2.

6. A nucleic acid encoding the TNA polymerase of claim 1.

7. The nucleic acid of claim 6, which is an expression vector.

8. A recombinant cell comprising the nucleic acid expression vector of claim 7.

9. The recombinant cell of claim 8, wherein the recombinant cell is a prokaryotic cell.

10. A kit comprising the TNA polymerase of claim 1 and at least one threose nucleotide.

11. The kit of claim 10, wherein the at least one threose nucleotide comprises tA, tT, tG, and tC.

12. A method for synthesizing a TNA, the method comprising contacting a DNA template with:
(i) a TNA polymerase of claim 1; and
(ii) a plurality of threose nucleotide triphosphates;
under conditions that permit TNA polymerization, whereby a TNA is synthesized by the TNA polymerase.

13. The method of claim 12, wherein the amino acid sequence is at least 90% identical to SEQ ID NO: 2.

14. A method for synthesizing TNA oligonucleotides, the method comprising contacting a DNA template with:
(i) a TNA polymerase of claim 1; and
(ii) a plurality of threose nucleotide triphosphates, wherein at least one threose nucleotide triphosphate comprises a modified base;
under conditions that permit TNA polymerization, whereby a TNA comprising a modified base is synthesized by the TNA polymerase.

15. The method of claim 14, wherein the amino acid sequence is at least 90% identical to SEQ ID NO: 2.

16. The method of claim 14, wherein the modified base comprises a C5-modified tUTP or tCTP monomer or C7-modified 7-deaza-tGTP or 7-deaza-tATP monomer bearing side chains.

17. The method of claim 16, wherein the side chains are selected from phenylalanine (Phe), tryptophan (Trp), tyrosine, cyclopropyl, naphthalene, and isoleucine.
